(19) United States
(12) Patent Application Publication
(10) Pub. No.: US 2024/0167043 A1
(43) Pub. Date: May 23, 2024

(54) COMPOSITIONS AND METHODS FOR ENHANCING BIOMASS PRODUCTIVITY IN PLANTS

(71) Applicant: Living Carbon PBC, Hayward, CA (US)

(72) Inventors: Madeline HALL, Hayward, CA (US); Li-Wei CHIU, Hayward, CA (US); Rebecca DEWHIRST, Hayward, CA (US); Jacob HOYLE, Hayward, CA (US); Patrick MELLOR, Hayward, CA (US); Karli RASMUSSEN, Hayward, CA (US); Yumin TAO, Hayward, CA (US)

(21) Appl. No.: 18/434,455

(22) Filed: Feb. 6, 2024

Related U.S. Application Data
(63) Continuation of application No. 18/153,863, filed on Jan. 12, 2023, now Pat. No. 11,926,833.
(60) Provisional application No. 63/302,895, filed on Jan. 25, 2022.

(51) Int. Cl. C12N 15/52; C12N 15/113

(52) U.S. Cl. CPC C12N 15/52; C12N 15/113; C12N 2310/11

(57) ABSTRACT

The present disclosure relates to a transgenic plant cell comprising polynucleotide sequences encoding glycolate dehydrogenase, malate synthase, and an inhibitory polynucleotide targeting an endogenous glycolate transporter Plgg1, wherein expression of endogenous glycolate transporter Plgg1 in the transgenic plant cell is about 20% to 80% of expression of endogenous glycolate transporter Plgg1 in a plant cell that is not transformed with an inhibitory polynucleotide targeting an endogenous glycolate transporter Plgg1. Also disclosed are transgenic plants, transgenic plant cultures, and methods for increasing photosynthesis efficiency in plants. The disclosed methods enhance biomass productivity and reduce the negative impact of photorespiration and introduction of transgenic constructs on plant growth.

This application is a continuation of U.S. patent application Ser. No. 18/153,863, filed Jan. 12, 2023, which claims the priority benefit of U.S. Provisional Patent Application Ser. No. 63/302,895, filed Jan. 25, 2022, which are hereby incorporated by reference in their entirety.

### FIELD

The present disclosure relates to compositions and methods for enhancing biomass productivity and decreasing the negative impact of photorespiration in plants.

### SEQUENCE LISTING

The Sequence Listing is being submitted electronically in XML format and is hereby incorporated by reference in its entirety. Said XML copy, created on Jan. 12, 2023, is named 257948000133.xml and is 100 KB in Size.

### BACKGROUND

Over the last century, the burning of fossil fuels such as coal and oil has increased the concentration of carbon dioxide in the atmosphere, with consequent global warming and shift in climate patterns.

Reforestation is, in theory, a low-cost and effective method to decrease carbon dioxide concentration in the atmosphere. However, reforestation is limited by the increase in global food demand, which elevates the need for cropland, and also by political considerations. Carbon drawdown and wood production in forest trees both depend on growth rate. Genetic transformations are able to increase the durability of wood, but they could also possibly reduce growth rate. Pressure treatment solutions used for wood are expensive, toxic, and greatly increase the weight of treated wood without improving its structural properties. There is therefore a need to stimulate growth rate of forest trees.

Fertilizers and pesticides are routinely applied to plants to stimulate growth rate. However, attempts to increase photosynthetic carbon capture in plants have not proven successful in field trials.

Photorespiration is the process by which the enzyme ribulose-1,5-bisphosphate carboxylase/oxygenase (“RuBisCO”) in C3 plants uses oxygen to oxygenate ribulose bisphosphate (“RuBP”), rather than fixing carbon dioxide to produce 3-phosphoglycerate and 2-phosphoglycolate, and release oxygen. This process requires energy in the form of ATP and reducing NADPH molecules and, as a consequence, it greatly reduces photosynthesis efficiency. Photorespiration produces glycolate and releases carbon dioxide and ammonia into the atmosphere. Glycolate must be metabolized at high-energy cost to prevent ammonia from accumulating in the leaves. Because of its high-energy cost, the photorespiration process significantly reduces biomass productivity and increases the release of carbon dioxide into the atmosphere. CO₂ emission into the atmosphere contributes to the “greenhouse effect” that has steadily increased in recent years, with consequent warming of the planet. There is a need to increase photosynthetic efficiency and biomass productivity in plants, and especially in trees.

The present disclosure is directed to overcoming these and other deficiencies in the art.

### SUMMARY

One aspect of the present disclosure relates to a transgenic plant cell comprising a first promoter operably linked to a polynucleotide sequence encoding a glycolate dehydrogenase, a second promoter operably linked to a polynucleotide sequence encoding a malate synthase, and an inhibitory polynucleotide targeting an endogenous glycolate transporter Plgg1, where expression of the endogenous glycolate transporter Plgg1 in the transgenic plant cell is about 20% to 80% of expression of endogenous glycolate transporter Plgg1 in a plant cell that is not transformed with an inhibitory polynucleotide targeting an endogenous glycolate transporter Plgg1.

Another aspect of the present disclosure relates to a transgenic plant comprising a transgenic plant cell of the present disclosure.

A further aspect of the present disclosure relates to a polynucleotide construct comprising an inhibitory polynucleotide comprising a polynucleotide sequence that targets an endogenous glycolate transporter Plgg1 present in a plant cell, wherein when expressed in the plant cell, the inhibitory polynucleotide reduces expression of the endogenous glycolate transporter Plgg1 by about 20% to 80% of expression of endogenous glycolate transporter Plgg 1 in a plant cell where the inhibitory polynucleotide is not present.

Another aspect of the present disclosure relates to a method for increasing biomass productivity in a plant. This method involves transforming a plant cell with one or more polynucleotides comprising a first promoter operably linked to a polynucleotide sequence encoding a glycolate dehydrogenase, a second promoter operably linked to a polynucleotide sequence encoding a malate synthase, and an inhibitory polynucleotide targeting an endogenous glycolate transporter Plgg1. Expression of the endogenous glycolate transporter Plgg1 in the transformed plant cell is about 20% to 80% of expression of endogenous glycolate transporter Plgg1 in a plant cell that is not transformed with an inhibitory polynucleotide targeting an endogenous glycolate transporter Plgg1 and said transforming increases biomass productivity of the plant.

Another aspect of the present disclosure relates to a transgenic plant cell culture produced by methods described herein.

A further aspect of the present disclosure relates to a method for increasing photosynthetic activity and stomatal conductance under drought conditions in a plant. This method involves transforming a plant cell with one or more polynucleotides comprising a first promoter operably linked to a polynucleotide sequence encoding a glycolate dehydrogenase, a second promoter operably linked to a polynucleotide sequence encoding a malate synthase, and an inhibitory polynucleotide targeting an endogenous glycolate transporter Plgg1, where expression of the endogenous glycolate transporter Plgg1 in the transgenic plant cell is about 20% to 80% of expression of endogenous glycolate transporter Plgg1 in a plant cell that is not transformed with an inhibitory polynucleotide targeting an endogenous glycolate transporter Plgg1. The method further involves regenerating a plant from the transformed plant cell, and subjecting the plant to drought conditions. Said transforming increases photosynthetic activity and stomatal conductance under drought conditions in the regenerated plant.

The present disclosure relates to compositions (e.g., polynucleotide constructs, transgenic plant cells, plants containing polynucleotide constructs and transgenic plant cells, and transgenic plant cell cultures) and methods for increasing photosynthesis efficiency and/or biomass productivity in plants. Also provided are compositions (e.g., polynucleotide constructs, transgenic plant cells, plants containing polynucleotide constructs and transgenic plant cells, and transgenic plant cell cultures) and methods for increasing photosynthetic activity and/or stomatal conductance in plants. In some embodiments, such plants are growing or are expected to grow under drought conditions.

Compositions and methods of the present disclosure are designed or engineered to induce glycolate metabolism and by inhibiting glycolate transport in the photorespiratory pathway, thereby decreasing the negative impact of photorespiration on plant growth and offsetting any slowdown in growth, which may be caused by incorporation of transgenic constructs into plants. The negative impact of photorespiration on the growth of plants, including tree plants, can be significantly decreased by engineering the plants to reduce the transportation of glycolate out of chloroplasts and to metabolize the retained glycolate to CO₂ for RuBisCo enzyme to fix inside chloroplasts.

Based on findings described in the Examples infra, the present disclosure provides compositions and methods to enhance biomass productivity and carbon uptake in plants by increasing their photosynthetic efficiency and reducing photorespiration losses, such that negative impacts on plant growth caused by photorespiration and introduction of transgenic constructs is reduced.

Moreover, the disclosed compositions and methods described herein make use of inhibitory polynucleotides to down-regulate, but not eliminate or severely reduce, the expression of the native chloroplast glycolate transporter Plgg1 in the photorespiratory pathway, thus limiting glycolate efflux from the chloroplasts. U.S. Patent Application Publication No. 2018/0258440 to Ort et al., co-expressed a malate synthase and glycolate dehydrogenase with an RNAi construct against glycolate transporter Plgg1 in tobacco plants, and showed that knockdown and knockout strains of glycolate transporter Plgg1 function similarly. However, as described herein, it is surprisingly shown that the down-regulation of endogenous glycolate transporter Plgg1 expression by about 80% or more compared to the normal, wild-type expression or expression level of glycolate transporter Plgg1 in a control plant, leads to severe detrimental morphological effects on plants when co-expressed with malate synthase and glycolate dehydrogenase. A reduction of endogenous glycolate transporter Plgg1 expression by less than 20% had little effect on phenotype. However, a more moderate reduction of endogenous expression levels of glycolate transporter Plgg1 (i.e., a plant having about 20% to about 80% of the expression level of glycolate transporter Plgg1 relative to a wild-type control plant) improved growth rate and biomass productivity.

The compositions and methods disclosed herein led to plants and forest trees with increased biomass productivity of at least 10-40% or more, as well as increased photosynthetic activity and stomatal conductance under drought conditions.

### BRIEF DESCRIPTION OF THE DRAWINGS

FIG. 1 is a schematic illustration of Populus trichocarpa PtPlgg1 in its endogenous genomic context.

FIG. 2 is a schematic illustration of “trigger site one” represented on a post-transcriptional RNA model of the PtPlgg1 nucleotide sequence.

FIG. 3 is a schematic illustration of one embodiment of a PtPlgg1 RNAi cassette. The PtPlgg1 sense fragment and the PtPlgg1 antisense fragment are separated by the PDK intron.

FIG. 4 is a sequence alignment of Populus alba Plgg1 (PalbaPlgg1) (SEQ ID NO:38), Populus tremula (PtremPlgg1) (SEQ ID NO:39), and Populus trichocarpa Plgg1 (PtricPlgg1) (SEQ ID NO:6) coding regions, and the sense portion of the sequence used for RNAi (SEQ ID NO:8) in pLC0102.

FIG. 5 is a graph showing the siRNA Finder (“siFi”) output for RNAi design.

FIG. 6 shows the siFi output for off-target prediction in PtPlgg1, which allows three mismatches in 21mer siRNAs.

FIG. 7 is a schematic illustration showing one embodiment of a vector described herein, pLC0102, with polynucleotide constructs comprising the glycolate dehydrogenase gene from Chlamydomonas reinhardtii (“GDH”) operably linked to the Act2 promoter and the RbcS signal peptide, the malate synthase gene (“MS”) from Cucurbita hybrida (sp. Kurokawa Amakuri Nankin) operably linked to the Spm promoter and the RbcS signal peptide, and an RNAi for the glycolate transporter Plgg1 from Populus trichocarpa. Expression of the RNAi cassette is driven by the AtRbcs2b promoter.

FIG. 8 is a schematic illustration showing one embodiment of a vector described herein, with the RbcS signal peptide and transit peptidase cleavage sites added at the N-terminus of CrGDH and MS to target chloroplasts.

FIG. 9 is a photograph of regenerated transgenic Populus tremula x Populus alba shoots transformed with pLC0102.

FIG. 10 is a graph showing the expression level of Plgg1 in lead LC0102 events. Expression levels of events LC0102.5A, 7F, 8-9A, and 13-15E had reduced levels of Plgg1 ranging from 46% to 73% of Plgg1 expression in the control (wild-type). Tissue samples were collected from three ramets per event except for two ramets for event 13-15E. The symbols * and ** indicate statistically significant differences at p<0.05 and p<0.01, respectively, when compared to non-transgenic CT717.3. Statistical analysis was performed based on one-way ANOVA.

FIG. 11 is a graph of CO₂ assimilation curves, or A-Ci curves, measuring photosynthesis efficiency of 18 week old plants including non-transgenic 717 controls and two lead LC0102 events, 13-15E, and 7F. Number of ramets measured: 717 control plants, 5; 13-15E, 2; 7F, 3. Error bars indicate SEM.

FIG. 12 is a graph illustrating the average heights of triplicate control and selected transgenic LC0102 events measured 3 weeks after transfer to soil pots.

FIGS. 13A-B relate to plant height analysis of four-month-old plants of selected events LC0102.13-15E, 5A, 7F, and 8-9A in comparison to non-transgenic control plants 717. Number of ramets: 13-15E, 2; 5A, 3; 7F, 3; 8-9A, 3; Control 717, 5. FIG. 13A is a photograph showing the height difference between events and controls. Blue arrow: LC0102 events; Red arrow: non-transgenic control. FIG. 13B is a graph showing the average plant height measured at 17 weeks post potting. The symbols * and ** represent a significant difference at p<0.05 and p<0.01, respectively, when compared to non-transgenic 717 plants. Statistical analysis was performed based on one-way ANOVA.

FIGS. 14A-B are graphs showing the average fresh weight (FIG. 14A) and average dry weight (FIG. 14B) biomass production after 21 weeks of growth in controlled environments. Number of ramets: Event 13-15E, 2; Event 7F, 3; Non-transgenic control CT717, 5. AG: Above ground biomass=Leaf+Stem. Symbols * and ** represent significant differences at p<0.05 and p<0.01, respectively, when compared with non-transgenic CT717. Statistical analysis was performed based on one-way ANOVA.

FIGS. 15A-B are graphs showing stem volume index (mm³) measured approximately 1 year after transplantation in a field environment in July (FIG. 15A) and August (FIG. 15B) for various transgenic events (13-15B, 2H, 13-15E, 1, 5, 4B, 7, 1C, 4A, 5A, and 5C), non-transgenic controls (CT1 and CT3), and escapes (16-20, 8-9D (having no transgene)).

FIGS. 16A-B are graphs showing the Jₘₐₓ (maximum rate of photosynthetic electron transport) under no, mild, medium, or severe drought stress (FIG. 16A) and the Aₘₐₓ (maximum carbon assimilation rate) under no, mild, medium, or severe drought stress (FIG. 16B). Jₘₐₓ and Aₘₐₓ are indicators for photosynthetic activity in plants.

FIGS. 17A-B are graphs showing stomatal conductance under no, mild, medium, or severe drought stress (FIG. 17A) and water use efficiency (A/gsw; A, net carbon assimilation; gsw, stomatal conductance to water vapor) under no, mild, medium, or severe drought stress (FIG. 17B).

FIGS. 18A-C are graphs showing the relative expression levels of endogenous Plgg1 (FIG. 18A) and transgenes Glycolate Dehydrogenase (CrGDH) (FIG. 18B) and Malate Synthase (ChMS) (FIG. 18C) in various transgenic events and controls.

### DETAILED DESCRIPTION

The present disclosure relates to transgenic plant cells, transgenic plants, and methods for increasing biomass productivity, decreasing the negative impact of photorespiration, and increasing photosynthesis efficiency in plants.

Unless otherwise indicated, the definitions and embodiments described in this and other sections are intended to be applicable to all embodiments and aspects of the present disclosure herein described for which they are suitable as would be understood by a person of ordinary skill in the art.

Singular forms “a”, “an”, and “the” include plural references unless the context clearly dictates otherwise. Thus, for example, a reference to “a method” includes one or more methods, and/or steps of the type described herein and/or which will become apparent to a person of ordinary skill in the art upon reading the present disclosure.

The term “about” includes being within a statistically meaningful range of a value. Such a range can be within an order of magnitude, preferably within 0-10%, 10%, 5%, 4%, 3%, 2%, 1%, or at a given value or within a given range. The allowable variation encompassed by the term “about” depends on the particular context and as a person of ordinary skill in the art would appreciate.

The term “and/or” as used herein means that the listed items are present, or used, individually (e.g., as alternatives) or in combination. In effect, this term means that “at least one of” or “one or more” of the listed items is used or present.

In understanding the scope of the present disclosure, the term “comprising” and its derivatives, as used herein, are intended to be open ended terms that specify the presence of the stated features or method steps, but do not exclude the presence of other unstated features or method steps. The foregoing also applies to words having similar meanings such as the terms, “including”, “involving”, “having”, and their derivatives. The term “comprising” may be substituted with the term “consisting of” to specify the presence of the stated features or method steps only and to exclude the presence of other unstated features or method steps. The term “comprising” may alternatively be substituted with the term “consisting essentially of” to specify the presence of the stated features or method steps as well as those that do not materially affect the basic and novel characteristic(s) of features or method steps as a person of ordinary skill in the art would appreciate.

As used herein, the term “coding region” is a portion of a nucleic acid, which is transcribed and translated into a polypeptide or protein.

As used herein, the term “conservative substitution” is a mutation in amino acid sequence where one or more amino acids is substituted by another amino acid having highly similar properties. Examples include, but are not limited to, one or more amino acids comprising nonpolar or aliphatic side chains, such as glycine, alanine, valine, leucine, isoleucine, or proline, which can be substituted for one another; one or more amino acids comprising polar, uncharged side chains, such as serine, threonine, cysteine, methionine, asparagine, or glutamine, which can be substituted for each other; one or more amino acids comprising aromatic side chains, such as phenylalanine, tyrosine, or tryptophan, which can be substituted for each other; one or more amino acids comprising positively charged side chains, such as lysine, arginine, or histidine, which can be substituted for each other; one or more amino acids comprising negatively charged side chains, such as aspartic acid or glutamic acid, which can be substituted for each other. For example, conservative substitutions for leucine include alanine, isoleucine, valine, phenylalanine, tryptophan, methionine, and cysteine, and conservative substitutions for asparagine include arginine, lysine, aspartate, glutamate, and glutamine. Conservatively modified nucleic acids include nucleic acids that encode identical or essentially identical amino acid sequences.

A “gene” refers to an assembly of nucleotides that encode a polypeptide, and includes cDNA and genomic DNA nucleic acids. “Gene” also refers to a nucleic acid fragment that expresses a specific functional RNA, protein, or polypeptide, optionally including regulatory sequences preceding (5′ noncoding sequences) and following (3′ noncoding sequences) the coding sequence. “Native gene” refers to a gene as found in nature with its own regulatory sequences. “Chimeric gene” refers to any gene that is not a native gene, comprising regulatory and/or coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A chimeric gene may comprise coding sequences derived from different sources and/or regulatory sequences derived from different sources. “Endogenous gene” refers to a native gene in its natural location in the genome of an organism. A “foreign” gene, “heterologous” gene, or “exogenous” gene refers to a gene not normally found in the host organism, but that is introduced into the host organism by gene transfer. Heterologous genes can comprise native genes inserted into a non-native organism, or chimeric genes, such as a native gene under control of a different promoter than its endogenous promoter. A “transgene” is a gene that has been introduced into the genome by a transformation or transfection procedure.

“Heterologous” or “exogenous” DNA refers to DNA not naturally located in the cell, or in a chromosomal site of the cell. The heterologous DNA can include a gene or other polynucleotides foreign to the cell.

“Operably linked” means an association between polynucleotide (e.g., nucleic acid) sequences on a single nucleic acid molecule such that the function of one is affected by the other. For example, a promoter is operably linked to a coding sequence when the coding sequence is under the transcriptional control of the promoter. Coding sequences can be operably linked to regulatory sequences in sense or antisense orientation.

A “polypeptide” is a naturally occurring or synthetic peptide, oligopeptide, polypeptide, gene product, expression product, or protein comprising an amino acid sequence, where the amino acids are joined to each other by peptide bonds or modified peptide bonds.

A “promoter” refers to a nucleic acid fragment capable of controlling transcription of another nucleic acid fragment. A promoter is a non-coding genomic DNA sequence, usually upstream (5′) to and operably linked to the relevant coding sequence, and its primary function is to act as a binding site for RNA polymerase to initiate transcription by the RNA polymerase. A promoter may also include distal enhancer or repressor elements, which can be located as much as several thousand base pairs from the start site of transcription. The terms “capable of controlling expression” or “initiating transcription”, refer to the primary function of a promoter. Additionally, there is “expression” of RNA, including functional RNA, or the expression of polypeptide for operably linked encoding nucleotide sequences, as the transcribed RNA ultimately may be translated into the corresponding polypeptide. Promoters vary in their “strength” (i.e., their ability to promote transcription). The nucleotide sequence of the promoter determines the nature of the RNA polymerase binding and other related protein factors that attach to the RNA polymerase and/or promoter, and the rate of RNA synthesis.

A “constitutive” promoter is a promoter that is active under most environmental and developmental conditions. An “inducible” promoter is a promoter that is active under environmental or developmental regulation.

A “reference sequence” means a nucleic acid or amino acid used as a comparator for another nucleic acid or amino acid, respectively, when determining sequence identity. A reference sequence can be a wildtype sequence.

“Sequence identity,” “percent identity,” or “% identical” refers to the exactness of a match between a reference sequence and a sequence being compared to it when optimally aligned. For example, sequence alignments and percent identity calculations may be determined using a variety of comparison methods designed to detect homologous sequences including, but not limited to, the Multalin program (Corpet, “Multiple Sequence Alignment with Hierarchical Clustering,” Nucleic Acids Res. 16:10881-90 (1988), which is hereby incorporated by reference in its entirety) or the Megalign® program of the LASERGENE® bioinformatics computing suite (DNASTAR® Inc., Madison, Wis.). Sequences may also be aligned using algorithms known in the art including, but not limited to, CLUSTAL V algorithm or the BLASTN or BLAST 2 sequence programs.

As used herein, the term “plant cell” includes cells, protoplasts, cell tissue cultures from which plants can be regenerated, calli, clumps, and cells that are intact in plants or parts of plants including, but not limited to seeds, leaves, stems, roots, vegetative buds, floral buds, meristems, embryos, hypocotyls, cotyledons, endosperm, sepals, petals, pistils, carpels, stamens, anthers, microspores, pollen, pollen tubes, ovules, nucellar tissue, ovaries, and other plant tissue or cells. In some embodiments, the plant cell is a protoplast.

“Transformation” or “transforming” refers to the introduction of a nucleic acid into a host organism. Host organisms containing a transformed polynucleotide construct or DNA fragment are referred to as “transgenic” or “recombinant” organisms. “Transfection” refers to the introduction of a nucleic acid, a protein, or both into a host organism. Thus, isolated polynucleotides disclosed herein can be incorporated into recombinant constructs, typically polynucleotide constructs, capable of introduction into and replication in a host cell. A construct can be a vector that includes a replication system and sequences that are capable of transcription and translation of a polypeptide-encoding sequence in a given host cell. Typically, expression vectors include, for example, one or more cloned genes under the transcriptional control of 5′ and 3′ regulatory sequences and a selectable marker. Such vectors also can contain a promoter regulatory region, such as a regulatory region controlling inducible or constitutive, environmentally- or developmentally-regulated, or tissue-specific expression, a transcription initiation start site, a ribosome binding site, a transcription termination site, and/or a polyadenylation signal.

The phrase “under conditions effective” is used to describe any environment that permits the desired reaction to take place.

A “vector” or “polynucleotide construct” is a nucleic acid, plasmid, or virus used to transfer coding information to a host cell. A “cloning vector” is a small piece of DNA into which a foreign DNA fragment is inserted to be transcribed. Typically the vector contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. The DNA sequence in the expression vector is operably linked to appropriate expression control sequences, including a promoter, to direct RNA synthesis and protein expression. The expression vector can contain one or more selectable marker genes to provide a phenotypic trait for selection of a transformed host cell. Useful selectable markers include dihydrofolate reductase or neomycin resistance for eukaryotic cell culture, or tetracycline or ampicillin resistance in E. coli. The vector may be introduced into the host cell(s) using any of a variety of techniques, including transformation, transfection, transduction, viral infection, gene guns, Ti-mediated gene transfer, calcium phosphate transfection, DEAE-Dextran-mediated transfection, lipofection, or electroporation. Examples of vectors include, but are not limited to, viral particles, baculovirus, phage, plasmids, phagemids, cosmids, fosmids, bacterial artificial chromosomes, viral DNA, such as vaccinia and adenovirus, P1-based artificial chromosomes, yeast plasmids, Bacillus vectors, and Aspergillus vectors. Examples of bacterial vectors include, but are not limited to, pQE vectors, pBluescript plasmids, pNH vectors, and lambda-ZAP vectors. Examples of eukaryotic vectors include pXT1, pSG5, pSVK3, pBPV, pMSG, and pSVLSV40 vectors. Suitable constitutive promoters that are functional in a plant cell include, but are not limited to, the cauliflower mosaic virus 35S (CaMV35S) promoter, a tandem 35S promoter, a cauliflower mosaic virus 19S promoter, a figwart mosaic virus 35S (FMV35S) promoter, a nopaline synthase gene promoter, an octopine synthase gene promoter, a potato or tomato protease inhibitor I or II gene promoter, and a ubiquitin promoter. Suitable inducible promoters that are functional in a plant cell may include, but are not limited to, a phenylalanine ammonia-lyase gene promoter, a chalcone synthase gene promoter, a pathogenesis-related protein gene promoter, a copper-inducible regulatory element, tetracycline, and chlor-tetracycline-inducible regulatory elements.

Preferences and options for a given aspect, feature, embodiment, or parameter of the disclosure should, unless the context indicates otherwise, be regarded as having been disclosed in combination with any and all preferences and options for all other aspects, features, embodiments, and parameters of the disclosure.

### Compositions and Methods of Producing Photosynthesis-Efficient Plants

One aspect of the present disclosure relates to a transgenic plant cell comprising a first promoter operably linked to a polynucleotide sequence encoding a glycolate dehydrogenase, a second promoter operably linked to a polynucleotide sequence encoding a malate synthase, and an inhibitory polynucleotide targeting an endogenous (i.e., native) glycolate transporter Plgg1, where expression of the endogenous glycolate transporter Plgg1 in the transgenic plant cell is about 20% to 80% of expression of endogenous glycolate transporter Plgg1 in a plant cell that is not transformed with an inhibitory polynucleotide targeting an endogenous glycolate transporter Plgg1.

This aspect of the present disclosure can be carried out with any of the embodiments disclosed herein.

In the transgenic plant cell and other aspects of the present disclosure, glycolate dehydrogenase (“GDH”) is an enzyme, such as glycolate dehydrogenase from Chlamydomonas reinhardtii, which oxidates glycolate into glyoxylate using organic co-factors instead of oxygen as electron acceptors. In some embodiments, the polynucleotide sequence encodes a bacterial glycolate dehydrogenase or an algae glycolate dehydrogenase, such as a glycolate dehydrogenase from Chlamydomonas reinhardtii.

In some embodiments, the polynucleotide sequence encodes glycolate dehydrogenase from Chlamydomonas reinhardtii (“CrGDH”), which has a coding sequence of SEQ ID NO:1 (GenBank Accession No. XM_001695329.2, which is hereby incorporated by reference in its entirety) as follows:

ATGCCACGCG GCCAGGGCAA GCGCCTGGCT CAGCTCCTTG GAGCTCAGCT GAAGCAGTAC
GCAGCGGAGG TGCGTGGCAT CAGCACAGCT GGTGGCGCTT CTCGCGGTGG AGCTCGAGGA
CCTGCATCCC CTAGCTCGCT AGAGCAGCAG ACGCGCCAGG TCGCTCAGGT TGCTGTTCAG
CAGTCGACTC AGCAGGCAGT GAAGGTCGTT GTGCCGGCCA TCAAAGTAGA CCTGGTTGGT
GCGGTCAGCT CGGTGTCTGA GAGCGACAAG GTGGAGCCGG GTGTGTTCAA GAACGTGGAT
GGCCACCGCT TCGAGGACGG TCGCTATGCC GCTTTTGTTG AGGAGATTAC AAAGTTTATC
CCCAAGGAGC GCCAGTACTC GGACCCCGTG CGCACATTCG CGTATGGCAC GGATGCCTCC
TTCTACCGGC TTAACCCGAA GCTGGTAGTG AAGGTGCACA ACGAGGACGA GGTCCGCCGC
ATCATGCCCA TCGCGGAGCG GCTGCAGGTC CCTATCACCT TCCGCGCGGC CGGCACGTCG
CTGTCTGGGC AGGCAATTAC CGACTCGGTG CTCATTAAGC TGAGCCACAC GGGCAAGAAC
TTCCGCAACT TTACCGTGCA CGGCGACGGT AGCGTGATCA CGGTGGAGCC GGGCCTCATT
GGCGGCGAGG TGAACCGCAT CCTGGCGGCA CACCAGAAGA AGAACAAGCT GCCCATCCAG
TACAAGATCG GACCCGACCC CTCCTCCATC GACAGCTGCA TGATCGGCGG CATCGTGTCC
AACAACAGCA GCGGCATGTG CTGCGGCGTG AGCCAGAACA CCTACCACAC GCTGAAGGAC
ATGCGGGTGG TGTTCGTAGA CGGAACGGTG CTGGACACGG CCGACCCCAA CTCGTGCACC
GCCTTCATGA AGAGCCACCG CTCGCTGGTG GATGGCGTCG TGAGCCTGGC GCGCCGCGTG
CAGGCCGACA AGGAGCTGAC GGCGCTCATC CGCCGCAAGT TCGCCATCAA GTGCACCACC
GGCTACTCCC TGAACGCGCT GGTGGACTTC CCGGTGGACA ACCCCATTGA GATCATCAAG
CACCTCATCA TCGGCAGCGA GGGCACGCTG GGCTTCGTCA GCCGCGCCAC CTACAACACC
GTGCCCGAGT GGCCCAACAA GGCCTCGGCC TTCATCGTGT TCCCGGACGT GCGCGCCGCC
TGCACCGGCG CCTCGGTGCT GCGCAACGAG ACGTCCGTGG ACGCGGTGGA GCTGTTTGAC
CGCGCCAGCC TGCGCGAGTG CGAGAACAAC GAGGACATGA TGCGCCTGGT GCCCGACATC
AAGGGCTGCG ACCCCATGGC GGCAGCGCTG CTGATCGAGT GCCGCGGCCA GGACGAGGCC
GCACTGCAGA GCCGCATTGA GGAGGTGGTG CGCGTGCTGA CGGCGGCGGG CCTGCCCTTC
GGCGCCAAGG CCGCGCAGCC CATGGCCATC GACGCCTACC CCTTCCACCA CGACCAGAAG
AACGCCAAGG TCTTCTGGGA CGTGCGCAGG GGCCTGATCC CCATTGTGGG CGCGGCGCGC
GAGCCCGGCA CATCCATGCT GATCGAGGAC GTGGCCTGCC CCGTGGACAA GCTGGCCGAC
ATGATGATCG ACCTGATCGA CATGTTCCAG CGCCACGGCT ACCACGACGC CTCCTGCTTC
GGCCACGCGC TCGAGGGCAA CCTTCACTTG GTGTTCTCGC AGGGCTTCCG CAACAAGGAG
GAGGTGCAGC GCTTCAGCGA CATGATGGAG GAGATGTGCC ACCTGGTGGC CACCAAGCAC
TCGGGCAGCC TCAAGGGCGA GCACGGCACG GGCCGCAACG TGGCGCCGTT CGTGGAGATG
GAGTGGGGCA ACAAGGCGTA CGAGCTGATG TGGGAGCTCA AGGCGCTGTT CGACCCCAGC
CACACCCTCA ACCCGGGCGT CATCCTCAAC CGCGACCAGG ACGCGCACAT CAAGTTCCTG
AAGCCCTCGC CCGCGGCCTC GCCCATCGTC AACCGCTGCA TCGAGTGCGG CTTCTGCGAG
TCCAACTGCC CCTCGCGCGA CATCACGCTC ACGCCGCGCC AGCGCATCTC CGTGTACCGC
GAGATGTACC GCCTCAAGCA GCTGGGCCCG GGCGCCAGCG AGGAGGAGAA GAAGCAGCTG
GCGGCCATGA GCAGCTCGTA CGCCTACGAC GGCGAGCAGA CGTGCGCGGC GGACGGCATG
TGCCAGGAGA AGTGCCCCGT CAAGATCAAC ACGGGAGACC TGATCAAGTC GATGCGTGCC
GAGCACATGA AGGAGGAGAA GACCGCCAGC GGCATGGCAG ACTGGCTGGC CGCCAACTTC
GGCGTCATCA ACTCCAACGT GCCGCGCTTC CTCAACATCG TCAACGCCAT GCACAGCGTG
GTGGGCTCGG CGCCTCTGTC CGCCATCAGC CGCGCGCTCA ACGCCGCCAC CAACCACTTC
GTGCCGGTGT GGAACCCCTA CATGCCCAAG GGCGCGGCGC CGCTCAAGGT GCCCGCCCCG
CCGGCGCCGG CAGCTGCTGA GGCCTCGGGC ATCCCGCGCA AGGTGGTGTA CATGCCCAGC
TGCGTGACGC GCATGATGGG CCCCGCCGCC TCCGACACCG AGACGGCGGC GGTGCACGAG
AAGGTGATGA GCCTGTTCGG CAAGGCCGGC TACGAGGTGA TCATCCCCGA GGGCGTGGCC
AGCCAGTGCT GCGGCATGAT GTTCAACAGC CGCGGCTTCA AGGACGCCGC CGCCAGCAAG
GGCGCGGAGC TGGAGGCGGC GCTGCTCAAG GCCTCGGACA ATGGCAAGAT CCCCATCGTC
ATCGACACCT CGCCCTGCCT GGCGCAGGTG AAGAGCCAGA TCAGCGAGCC GTCGCTGCGC
TTCGCGCTGT ACGAGCCGGT TGAGTTCATC CGGCACTTCC TGGTGGACAA GCTGGAGTGG
AAGAAGGTGC GCGACCAGGT GGCCATCCAC GTGCCCTGCT CCTCCAAGAA GATGGGCATC
GAGGAGTCCT TCGCGAAGCT GGCGGGCCTG TGCGCCAACG AGGTGGTGCC CTCGGGCATT
CCTTGCTGCG GCATGGCGGG CGACCGCGGC ATGCGCTTCC CCGAGCTGAC CGGCGCCTCG
CTGCAGCACC TCAACCTGCC CAAGACCTGC AAGGACGGCT ACTCCACCAG CCGCACCTGC
GAGATGTCGC TCAGCAACCA CGCCGGCATC AACTTCAGGG GCCTGGTGTA CCTGGTGGAT
GAGGCCACGG CGCCTAAGAA GCAGGCCGCC GCTGCCAAGA CCGCGTAA

The amino acid sequence for CrGDH (SEQ ID NO:2) (GenBank Accession No. XP_001695381.1. which is hereby incorporated by reference in its entirety) is as follows:

MPRGQGKRLA QLLGAQLKQY AAEVRGISTA GGASRGGARG PASPSSLEQQ TRQVAQVAVQ
QSTQQAVKVV VPAIKVDLVG AVSSVSESDK VEPGVFKNVD GHRFEDGRYA AFVEEITKFI
PKERQYSDPV RTFAYGTDAS FYRLNPKLVV KVHNEDEVRR IMPIAERLQV PITFRAAGTS
LSGQAITDSV LIKLSHTGKN FRNFTVHGDG SVITVEPGLI GGEVNRILAA HQKKNKLPIQ
YKIGPDPSSI DSCMIGGIVS NNSSGMCCGV SQNTYHTLKD MRVVFVDGTV LDTADPNSCT
AFMKSHRSLV DGVVSLARRV QADKELTALI RRKFAIKCTT GYSLNALVDF PVDNPIEIIK
HLIIGSEGTL GFVSRATYNT VPEWPNKASA FIVFPDVRAA CTGASVLRNE TSVDAVELFD
RASLRECENN EDMMRLVPDI KGCDPMAAAL LIECRGQDEA ALQSRIEEVV RVLTAAGLPF
GAKAAQPMAI DAYPFHHDQK NAKVFWDVRR GLIPIVGAAR EPGTSMLIED VACPVDKLAD
MMIDLIDMFQ RHGYHDASCF GHALEGNLHL VFSQGFRNKE EVQRFSDMME EMCHLVATKH
SGSLKGEHGT GRNVAPFVEM EWGNKAYELM WELKALFDPS HTLNPGVILN RDQDAHIKFL
KPSPAASPIV NRCIECGFCE SNCPSRDITL TPRQRISVYR EMYRLKQLGP GASEEEKKQL
AAMSSSYAYD GEQTCAADGM CQEKCPVKIN TGDLIKSMRA EHMKEEKTAS GMADWLAANF
GVINSNVPRF LNIVNAMHSV VGSAPLSAIS RALNAATNHF VPVWNPYMPK GAAPLKVPAP
PAPAAAEASG IPRKVVYMPS CVTRMMGPAA SDTETAAVHE KVMSLFGKAG YEVITPEGVA
SQCCGMMFNS RGFKDAAASK GAELEAALLK ASDNGKIPIV IDTSPCLAQV KSQISEPSLR
FALYEPVEFI RHFLVDKLEW KKVRDQVAIH VPCSSKKMGI EESFAKLAGL CANEVVPSGI
PCCGMAGDRG MRFPELTGAS LQHLNLPKTC KDGYSTSRTC EMSLSNHAGI NFRGLVYLVD
EATAPKKQAA AAKTA

In some embodiments, the polynucleotide sequence encodes glycolate dehydrogenase from Chlamydomonas reinhardtii (CrGDH) having an amino acid sequence of SEQ ID NO:2. In some embodiments, the polynucleotide sequence encodes glycolate dehydrogenase having an amino acid sequence that is at least 80% identical to the amino acid sequence of CrGDH of SEQ ID NO:2. In some embodiments, the polynucleotide sequence encodes glycolate dehydrogenase comprising an amino acid sequence that is at least 80%, 83%, 85%, 90%, 91%, 92%, 93%, 94% 95%, 96%, 97%, 98%, 99%, or 100% identical to the amino acid sequence of CrGDH of SEQ ID NO:2. Additional exemplary glycolate dehydrogenase sequences that may be encoded by the polynucleotide sequence encoding a glycolate dehydrogenase are shown in Table 9 infra.

The transgenic plant cell and other aspects of the present disclosure also make use of the enzyme malate synthase (“MS”), such as malate synthase from the plant genus Cucurbita, which converts glyoxylate to malate, with consequent release of carbon dioxide in the chloroplast without causing toxic accumulation of ammonia. In some embodiments, the polynucleotide sequence encoding malate synthase encodes malate synthase from a plant of the genus Cucurbita. In some embodiments, the polynucleotide sequence encoding malate synthase encodes a malate synthase from the species Cucurbita hybrida (ChMS), such as Cucurbita sp. Kurokawa Amakuri Nankin. The coding sequence for one embodiment of the enzyme MS, ChMS (SEQ ID NO:3) (GenBank Accession No. X56948.1, which is hereby incorporated by reference in its entirety) is as follows:

ATGGGATCGC TGGGAATGTA TTCTGAATCG GCAGTAAGGA AGAAAAGTAG CCGAGGCTAC
GATGTTCCAG AGGGAGTGGA CATTCGGGGA CGTTATGATG AAGAATTTGC CAGGATTCTC
AACAAGGAAG CCTTGCTGTT TGTGGCTGAT TTACAGAGGA CTTTCAGAAA CCACATAAGG
TATTCGATGG AATGCCGCAG AGAAGCCAAA AGGAGGTACA ATGAAGGGGC GGTGCCGGGG
TTTGATCCGG CGACCAAGTA TATAAGGGAA TCTGAGTGGA CATGTGCATC AGTCCCCCCG
GCAGTTGCTG ATCGGAGAGT GGAGATCACC GGACCTGTGG AGCGGAAGAT GATCATCAAC
GCACTCAATT CTGGAGCTAA AGTTTTCATG GCGGACTTTG AAGATGCACT ATCACCAAAC
TGGGAGAATT TGATGAGGGG GCAAATTAAT CTGAAGGATG CAGTTGATGG GACTATAAGC
TTCCATGACA AAGCTAGAAA CAAGGTTTAT AAACTGAACG ATCAGACAGC CAAGCTCTTT
GTTCGCCCTC GAGGTTGGCA CTTCGCTGAG GCTCATATCT TCATCGACGG CGAGCCTGCC
ACCGGCTGTC TTGTGGACTT CGGGCTCTAC TTTTTTCACA ACCATGCCAA TTTCCGGCGC
TCTCAAGGTC AAGGTTCTGG CCCTTTCTTT TACCTTCCCA AAATGGAGCA CTCCAGGGAA
GCAAAAATAT GGAACAGTGT ATTTGAGAGA GCAGAGAAGA TGGCAGGGAT AGAGAGGGGC
AGCATCAGGG CCACTGTGCT GATTGAAACA CTTCCAGCAG TGTTTCAAAT GGATGAAATA
CTCTATGAGC TGAGGGATCA TTCTGTGGGA TTGAACTGTG GTAGATGGGA TTACATATTC
AGCTATGTCA AGACCTTCCA GGCTCACCTA GATCGCCTGT TACCCGACCG AGTCCAAGTC
GGTATGGCAC AACATTTCAT GAGGAGTTAT TCTGATCTCC TTATCAGGAC TTGTCACACG
GTGGTGTGCC ACGTGGGAGG CATGGCTGCT CAAATTCCAA TTAGAGACGA CCCGAAGGCA
AATGAGATGG CACTTGAGCT AGTGAGGAAG GACAAATTGA GAGAGGCAAA GGCAGGACAT
GATGGAACAT GGGCAGCACA TCCAGGATTA ATCCCAGCAT GTATGGAAGT CTTCACCAAC
AGCATGGGAA ATGCCCCCAA TCAGATCCGA TCTGCAAGAC GAGACGATGC TGCAAACCTA
ACTGAAGACG ACCTCTTGCA GCAACCGAGG GGTGTTCGTA CATTGGAAGG GCTCCGGTTG
AACACCCGAG TCGGAATTCA GTACCTAGCA GCATGGCTAA CCGGGACAGG CTCTGTGCCT
CTCTACAACC TTATGGAAGA TGCAGCCACA GCTGAAATCA GCAGGGTTCA AAACTGGCAA
TGGCTGAAGT ATGGAGTGGA ATTGGATGGA GATGGGCTTG GAGTGAGAGT GAACAAGGAA
CTGTTCGCAA GAGTGGTGGA AGAAGAAATG GAAAGGATTG AAAGAGAAGT GGGGAAGGAG
AAATTCAGGA AGGGAATGTA CAAAGAGGCT TGCAAGATGT TCACAAGGCA ATGCACAGCG
CCAACCTTGG ATGATTTTCT GACCTTGGAT GCGTACAACC ACATAGTCAT ACATCATCCC
AGGGAGCTGT CCAGGCTCTG A

The amino acid sequence for ChMS (SEQ ID NO:4) (GenBank Accession No. CAA40262, which is hereby incorporated by reference in its entirety) is as follows:

MGSLGMYSES AVRKKSSRGY DVPEGVDIRG RYDEEFARIL NKEALLFVAD LQRTFRNHIR
YSMECRREAK RRYNEGAVPG FDPATKYIRE SEWTCASVPP AVADRRVEIT GPVERKMIIN
ALNSGAKVFM ADFEDALSPN WENLMRGQIN LKDAVDGTIS FHDKARNKVY KLNDQTAKLF
VRPRGWHFAE AHIFIDGEPA TGCLVDFGLY FFHNHANFRR SQGQGSGPFF YLPKMEHSRE
AKIWNSVFER AEKMAGIERG SIRATVLIET LPAVFQMDEI LYELRDHSVG LNCGRWDYIF
SYVKTFQAHL DRLLPDRVQV GMAQHFMRSY SDLLIRTCHT VVCHVGGMAA QIPIRDDPKA
NEMALELVRK DKLREAKAGH DGTWAAHPGL IPACMEVFTN SMGNAPNQIR SARRDDAANL
TEDDLLQQPR GVRTLEGLRL NTRVGIQYLA AWLTGTGSVP LYNLMEDAAT AEISRVQNWQ
WLKYGVELDG DGLGVRVNKE LFARWEEEM ERIEREVGKE KFRKGMYKEA CKMFTRQCTA
PTLDDFLTLD AYNHIVIHHP RELSRL

In some embodiments, the polynucleotide sequence encodes malate synthase from Cucurbita hybrida (ChMS) having an amino acid sequence of SEQ ID NO:4. In some embodiments, the polynucleotide sequence encodes MS having an amino acid sequence that is at least 80% identical to the amino acid sequence of ChMS of SEQ ID NO:4. In some embodiments, the polynucleotide sequence encodes malate synthase comprising an amino acid sequence that is at least 80%, 83%, 85%, 90%, 91%, 92%, 93%, 94% 95%, 96%, 97%, 98%, 99%, or 100% identical to the amino acid sequence of ChMS of SEQ ID NO:4. Additional exemplary malate synthase sequences that may be encoded by the polynucleotide sequence encoding malate synthase are shown in Table 9 infra.

As noted above, the polynucleotide sequences encoding a glycolate dehydrogenase or a malate synthase are operably linked to a promoter. Exemplary promoters suitable for use include those that are inducible, viral, synthetic, or constitutive, all of which are well known in the art.

Non-limiting examples of promoters that may be useful in particular embodiments include the parsley ubiquitin promoter (Kawalleck et al. (1993) Plant Mol Biol 21: 673-684), which is hereby incorporated by reference in its entirety), Arabidopsis HSP70 promoter (Sung et al. (2001) Plant Physiol 126: 789-800, which is hereby incorporated by reference in its entirety), nopaline synthase (NOS) promoter (Ebert et al. (1987) Proc. Natl. Acad. Sci. USA 84(16):5745-9, which is hereby incorporated by reference in its entirety); the octopine synthase (OCS) promoter (which is carried on tumor-inducing plasmids of Agrobacterium tumefaciens); the caulimovirus promoters such as the cauliflower mosaic virus (CaMV) 19S promoter (Lawton et al. (1987) Plant Mol. Biol. 9:315-24, which is hereby incorporated by reference in its entirety); the CaMV 35S promoter (Odell et al. (1985) Nature 313:810-2; U.S. Pat. Nos. 5,322,938, 5,352,605, 5,359,142, and 5,530,196, which are hereby incorporated by reference in their entirety); the figwort mosaic virus 35S (FMV35S) promoter (Walker et al. (1987) Proc. Natl. Acad. Sci. USA 84(19):6624-8; U.S. Pat. Nos. 6,051,753, and 5,378,619, which are hereby incorporated by reference in their entirety); the sucrose synthase promoter (Yang and Russell (1990) Proc. Natl. Acad. Sci. USA 87:4144-8, which is hereby incorporated by reference in its entirety); the R gene complex promoter (Chandler et al. (1989) Plant Cell 1:1175-83, which is hereby incorporated by reference in its entirety); the chlorophyll a/b binding protein gene promoter; a PCI SV promoter (U.S. Pat. No. 5,850,019, which is hereby incorporated by reference in its entirety); the SCP1 promoter (U.S. Pat. No. 6,677,503, which is hereby incorporated by reference in its entirety); and AGRtu.nos promoters (GenBank Accession No. V00087; Depicker et al. (1982) J. Mol. Appl. Genet. 1:561-73; Bevan et al. (1983) Nature 304:184-7, which are hereby incorporated by reference in their entirety).

In some embodiments, a promoter may comprise a tissue-specific promoter. A tissue-specific promoter is a nucleotide sequence that directs a higher level of transcription of an operably linked nucleic acid in the tissue for which the promoter is specific, relative to the other tissues of the organism. Examples of tissue-specific promoters include, without limitation: tapetum-specific promoters; anther-specific promoters; pollen-specific promoters (see, e.g., U.S. Pat. No. 7,141,424, and PCT International Patent Publication No. WO 99/042587, which are hereby incorporated by reference in their entirety); ovule-specific promoters (see, e.g., U.S. Patent Application No. 2001/047525 A1, which is hereby incorporated by reference in its entirety); fruit-specific promoters (sec, e.g., U.S. Pat. Nos. 4,943,674, and 5,753,475, which are hereby incorporated by reference in their entirety); and seed specific promoters (see, e.g., U.S. Pat. Nos. 5,420,034, and 5,608,152, which are hereby incorporated by reference in their entirety). In some embodiments, a developmental stage—specific promoter (e.g., a promoter active at a later stage in development) may be used to control expression of a polynucleotide.

In some embodiments, the polynucleotide sequence encoding a glycolate dehydrogenase is operably linked to a first promoter. In some embodiments, the first promoter is a constitutive promoter. In some embodiments, the constitutive promoter is an Act2 (actin 2) promoter or a Cauliflower mosaic virus 35S promoter. In some embodiments, the first promoter is a suppressor-mutator (Spm) transposable element promoter.

In some embodiments, the polynucleotide sequence encoding a malate synthase is operably linked to a second promoter. In some embodiments, the second promoter is a constitutive promoter, such as an Act2 promoter, or a Cauliflower mosaic virus 35S promoter. In some embodiments, the second promoter is a suppressor-mutator (Spm) transposable element promoter.

In some embodiments, the polynucleotide sequence encoding the glycolate dehydrogenase is operably linked to a chloroplast-targeting signal sequence at the amino-terminus. In some embodiments, the polynucleotide sequence encoding a malate synthase is operably linked to a chloroplast-targeting signal sequence at the amino-terminus.

In some embodiments, the chloroplast-targeting signal sequence encodes a ribulose-1,5-bisphosphate carboxylase-oxygenase (RuBisCO) small subunit RbcS signal sequence.

The transgenic plant cell and other aspects of the present disclosure also comprise an inhibitory polynucleotide targeting an endogenous glycolate transporter Plgg1 to reduce its expression. Methods for reducing gene expression involving the expression of DNA sequences in plants are known in the art, and include, but are not limited to, cosuppression, antisense suppression, double-stranded RNA (dsRNA) interference, hairpin RNA (hpRNA) interference, intron-containing hairpin RNA (ihpRNA) interference, transcriptional gene silencing, and micro RNA (miRNA) interference.

For example, an “inhibitory polynucleotide” may be an antisense polynucleotide or an RNA inhibitor (RNAi) polynucleotide capable of down-regulating gene expression of a native or endogenous chloroplast glycolate transporter Plgg1 in the photorespiratory pathway, thus limiting glycolate efflux from chloroplasts in the transgenic plant cell.

RNA interference (RNAi) occurs when an organism recognizes double-stranded RNA molecules and hydrolyzes them. The resulting hydrolysis products are small RNA fragments of 19-24 nucleotides in length, called small interfering RNAs (siRNAs) or microRNAs (miRNAs). The siRNAs then hybridize to mRNAs (or other RNAs) and cause hydrolysis of the RNA. Most plant miRNAs show extensive base pairing to, and guide cleavage of their target mRNAs.

The RNAi pathway can be exploited in plants by using recombinant technology, which entails transforming a plant with a vector comprising DNA that when expressed produces a dsRNA homologous or nearly homologous to a gene target. The gene target can be homologous to an endogenous plant RNA. RNA interference in plants can also be referred to as post-transcriptional gene silencing or RNA silencing, and can be triggered by expression of an antisense strand of a target sequence of interest. In general, a plant is transformed with DNA that is incorporated into the plant genome, and when expressed produces a dsRNA that is complementary to a gene of interest, which can be an endogenous plant gene, leading to the reduction or elimination of expression of that gene.

In some embodiments, the inhibitory polynucleotide is an RNAi polynucleotide comprising a sense polynucleotide strand comprising at least 20 contiguous nucleotides from a glycolate transporter Plgg1 sequence, and an antisense polynucleotide strand that hybridizes to the sense polynucleotide strand, where the sense and the antisense polynucleotide strand form a duplex. In some embodiments, the RNAi polynucleotide comprises a sense polynucleotide strand comprising at least 20, 30, 40, 50, 60, 70, 80, 90, 100, 200, 300, 400 or more than 400 contiguous nucleotides from a glycolate transporter Plgg1 sequence, and an antisense polynucleotide strand that hybridizes to the sense polynucleotide strand, wherein the sense and the antisense polynucleotide strand form a duplex. In some embodiments, the RNAi polynucleotide comprises a sense polynucleotide strand 20-400 contiguous nucleotides from a glycolate transporter Plgg1 sequence, and an antisense polynucleotide strand that hybridizes to the sense polynucleotide strand, wherein the sense and the antisense polynucleotide strand form a duplex.

RNAi constructs may also include a polynucleotide sequence that separates the sense and antisense strands to facilitate duplex formation. In some embodiments, the RNAi polynucleotide further comprises a polynucleotide sequence heterologous to the sense and antisense polynucleotides. In some embodiments, the RNAi polynucleotide comprises an intron. In some embodiments, the RNAi polynucleotide comprises a PDK intron.

In some embodiments, the inhibitory polynucleotide comprises an antisense polynucleotide. In some embodiments, the inhibitory polynucleotide comprises an antisense polynucleotide of a glycolate transporter Plgg1 coding sequence of at least 20 base pairs (bp), 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, 100 bp, 125 bp, 150 bp, 175 bp, 200 bp, 300 bp, 400 bp, 500 bp, 600 bp, 700 bp, 800 bp, 900 bp, or greater than 1000 bp, or any number of base pairs or range of base pairs between 20 bp and 1000 bp.

In some embodiments, the glycolate transporter Plgg1 is selected from the group consisting of the genus Populus, Eucalyptus, Pinus, Quercus, Pseudotsuga, Picea, and Ficus, meaning that the transgenic cell is a cell of a plant of the genus Populus, Eucalyptus, Pinus, Quercus, Pseudotsuga, Picea, or Ficus, and the inhibitory polynucleotide targeting glycolate transporter Plgg1 targets glycolate transporter Plgg1 native or endogenous to a plant of the genus Populus, Eucalyptus, Pinus, Quercus, Pseudotsuga, Picea, or Ficus. In some embodiments, the glycolate transporter Plgg1 is from the genus Populus, meaning that the transgenic cell is a cell of a plant of the genus Populus and the inhibitory polynucleotide targeting glycolate transporter Plgg1 targets glycolate transporter Plgg1 native or endogenous to a plant of the genus Populus.

In some embodiments, the glycolate transporter Plgg1 is selected from the group consisting of the species Populus tremula x Populus alba, Populus alba, Populus tremula, Populus trichocarpa, Populus euphratica, and Populus tremuloides, meaning that the transgenic cell is a cell of a plant of the species Populus tremula x Populus alba, Populus alba, Populus tremula, Populus trichocarpa, Populus euphratica, or Populus tremuloides and the inhibitory polynucleotide targeting glycolate transporter Plgg1 targets glycolate transporter Plgg1 native or endogenous to one or more of the species Populus tremula x Populus alba, Populus alba, Populus tremula, Populus trichocarpa, Populus euphratica, or Populus tremuloides.

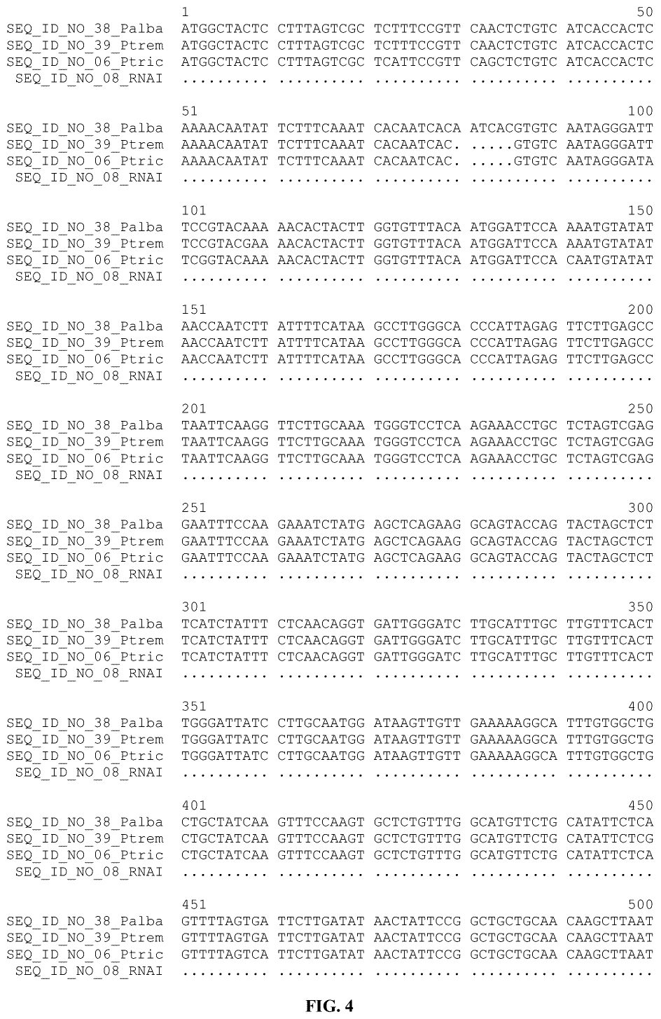

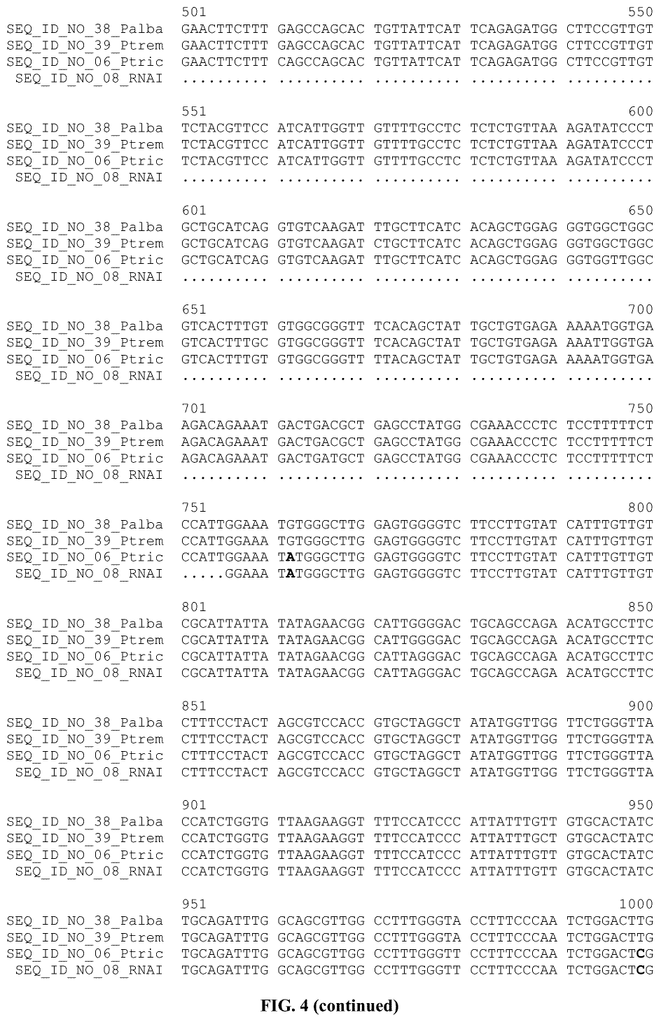

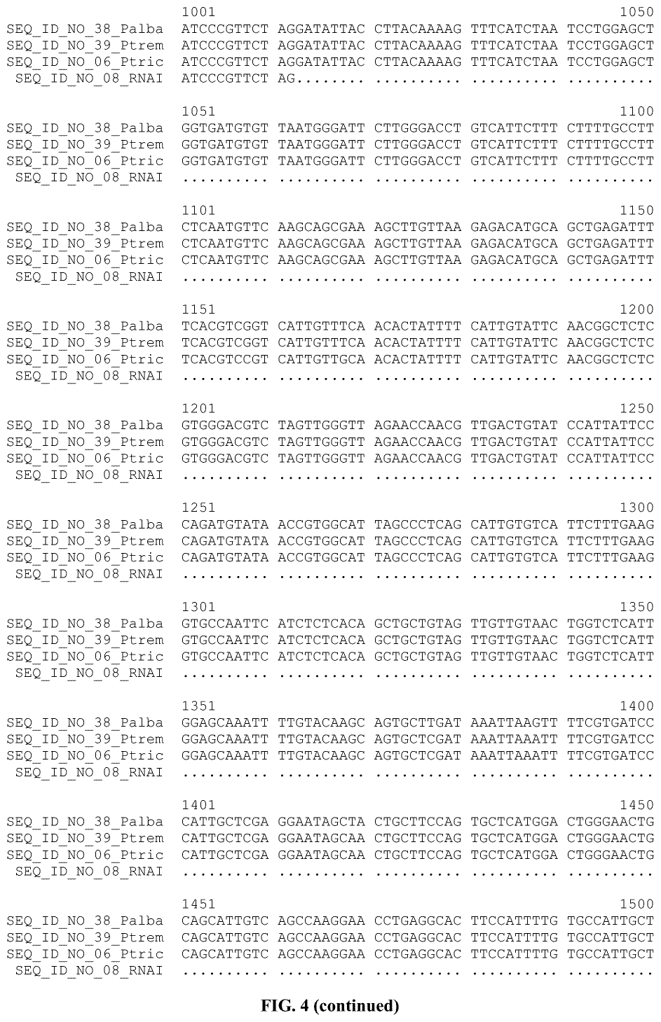

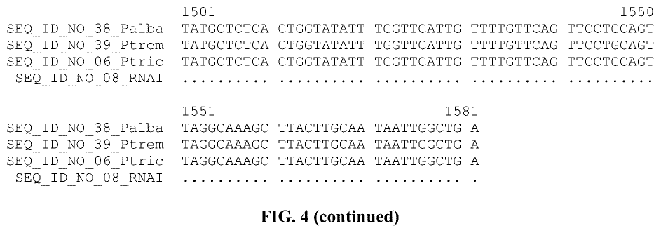

There is natural variation in the genetic sequence of the native chloroplast glycolate transporter Plgg1 coding sequences of different Populus varieties. In some embodiments, an inhibitory polynucleotide can reduce expression of glycolate transporter Plgg1 from different species. For example, FIG. 4 shows sequence variation between Plgg1 coding sequences of Populus trichocarpa, Populus tremula, and Populus alba. Even so, an inhibitory polynucleotide RNAi construct with Populus trichocarpa Plgg1 is able to reduce gene expression of Plgg1 in a Populus tremula x Populus alba hybrid (see Examples 1-11).

In some embodiments, the plant cell comprises two or more inhibitory polynucleotides targeting one or more endogenous glycolate transporter Plgg1 genes. For example, in a hybrid plant having two different endogenous glycolate transporter Plgg1 genes (one from each parent), it may be advantageous to use two inhibitory polynucleotides, each targeting and reducing the expression of a different endogenous glycolate transporter Plgg1 genes. Alternatively, it may be advantageous to have two or more inhibitory polynucleotides targeting the same endogenous glycolate transporter Plgg1 genes.

In some embodiments, the glycolate transporter Plgg1 is from the genus Pinus, meaning that the transgenic cell is a cell of a plant of the genus Pinus and the inhibitory polynucleotide targeting glycolate transporter Plgg1 targets glycolate transporter Plgg1 native or endogenous to a plant of the genus Pinus. In some embodiments, the glycolate transporter Plgg1 is from the species Pinus taeda, meaning that the transgenic cell is a cell of a plant of the species Pinus taeda and the inhibitory polynucleotide targeting glycolate transporter Plgg1 targets glycolate transporter Plgg1 native or endogenous to a plant of the species Pinus taeda.

In some embodiments, the inhibitory polynucleotide targeting an endogenous glycolate transporter Plgg1 comprises a polynucleotide sequence from Populus trichocarpa (“PtPlgg1”).

The genomic sequence of PtPlgg1 (SEQ ID NO:5) Potri.003G099600 (Chromosome 3:12562661-12568870, of the Populus trichocarpa genome (popgenie.org), which is hereby incorporated by reference in its entirety) is as follows, with the coding sequence indicated in bold font:

ATGGCTACTC CTTTAGTCGC TCATTCCGTT CAGCTCTGTC ATCACCACTC AAAACAATAT
TCTTTCAAAT CACAATCACG TGTCAATAGG GATATCGGTA CAAAAACACT ACTTGGTGTT
TACAATGGAT TCCACAATGT ATATAACCAA TCTTATTTTC ATAAGCCTTG GGCACCCATT
AGAGTTCTTG AGCCTAATTC AAGGTTCTTG CAAATGGGTC CTCAAGAAAC CTGCTCTAGT
CGAGGAATTT CCAAGAAATC TATGAGCTCA GAAGGCAGTA CCAGTACTAG CTCTTCATCT
ATTTCTCAAC AGGTTAGTAT AAAGGTTTTA CTGTTTTTCT TTTTTGCACT CCTTTTTATT
TTTTTTTATG CTTTCCTGTT TTGTATGATT TCTACTAATT GGTATTGAGT TGTTGCATTT
AATTAAGCTG ATTCAACTAT TTGATTTACC TCCTTTTTAT ATTGATTGCA TAAGGTGATT
GGGATCTTGC ATTTGCTTGT TTCACTTGGG ATTATCCTTG CAATGGATAA GTTGTTGAAA
AAGGCATTTG TGGCTGCTGC TATCAAGTTT CCAAGTGCTC TGTTTGGCAT GTTCTGCATA
TTCTCAGTTT TAGTCATTCT TGATATAACT ATTCCGGCTG CTGCAACAAG CTTAATGAAC
TTCTTTCAGC CAGCACTGTT ATTCATTCAG AGATGGCTTC CGTTGTTCTA CGTTCCATCA
TTGGTTGTTT TGCCTCTCTC TGTTAAAGAT ATCCCTGCTG CATCAGGTGT CAAGATTTGC
TTCATCACAG GTAAAACGAA GCTTTTTGTT GATTCATTGC TTTTCACTCT CTAAATCTTT
AATATAGTTG GCTTGATGAA TAAAATGTCA CTATGAATTT GTGATAATCA CACATAATTA
ATGTTATCTA TTGAAATCAC AGAAATTACT GCAAGCTCAA AAGCCAAGAT AACATAGCAT
CTCCTTCATT CTCCTTATGT TTCAAGTTTT TTGCTGACCA TTTCATTATT CATATCTCCG
ATTCCAATGG CCACCATTTT TTTTCTCACT TTGGTCATTA TTCATATACC ATCCAGTCAG
CATTCCCATG TTTACTTCCA TTTTGGTGGG ACTATTTTTC AAGTTGTGTT TTGCAATTGT
AATAAATAAT AATAATAATG ATGATACAAA TGCTTTCTAA TAACTGCAGC TGGAGGGTGG
TTGGCGTCAC TTTGTGTGGC GGGTTTTACA GCTATTGCTG TGAGAAAAAT GGTGAAGACA
GAAATGACTG ATGCTGAGCC TATGGCGAAA CCCTCTCCTT TTTCTCCATT GGAAATATGG
GCTTGGAGTG GGGTCTTCCT TGTATCATTT GTTGTCGCAT TATTATATAG AACGGCATTA
GGGACTGCAG CCAGAACATG CCTTCCTTTC CTACTAGCGT CCACCGTGCT AGGCTATATG
GTTGGTTCTG GGTAAAATCT CAGTTTCCTC AGATTAGCAA CCTCTGTGTT GACAATTGTT
TTCCAGACCA AATTGACGAG GAATGGAATT GTTGCACACA TTCATTATAA TTGTAGCCCA
GTTAAAAAAT CATGGCTTTA CTCTTCTGGT TTTCAGGTTA CCATCTGGTG TTAAGAAGGT
TTTCCATCCC ATTATTTGTT GTGCACTATC TGCAGATTTG GCAGCGTTGG CCTTTGGGTT
CCTTTCCCAA TCTGGACTCG ATCCCGTTCT AGGTACCACT TTTCAAAGTG TTTGCTGTCT
TCATTTAGTT CACTCATTTT CTGATGTGGA GCAACAACAA ATGACAACAA TTGTTACTAC
AAAATGCCTG TATTATTTTG TTCCACAAGC CATCTCTTGC TTTTCCTTTC TATCATATGT
GTTAGTTTCC ATTATGTATT ATGTTTCCTT CTGATACATG TAGTGTTAGT AGAGAACAAA
ACCTGGAGGG GCCATTTCAG GCAGTTTCCA TTGACAAACT TAGGTAGTGT TTGGTATTCC
GAACAAAGAT GGACCGGATA TGACAATATT TTTTGTGAAG TTGTTTTGTG TACTTTTGAA
TAGTACAAAA ATCAATTTTT ATCTTGTCCA AAGCTGTTCT AGTAGAGAAA ATTGTCTCAT
TAGAAGGGGT AGAACAACCT TGTCTAGTTC CTTGCAGAAG AGTTAACTTA GGTTGATCCG
AATCAGCATA AAATAAAAAT AGTTATTGTT ATAGTTATAG TTTTAAACTT GACTAGAGAG
TTGACTCAGG GCAAAGTTTG GGTTACGGAT TAGGTTGACT ATTGACTCAA GTCAATGTAA
GGATAAAAAT GTTTATTATC GTAGTTTTAA AACTTGACTT AGGGTCGAAC CGGGGCAAAG
CCCCGATCAC GGGTCGGGTT GAGTATTGAT CTAGGTCAAT GTAAGGATAA AATTATTGTT
ATCATAGCTT TAAAACCTGA CTAGGGGGTT GACCCGGGTC ACGGGTTGGT TGACCATTGA
GCCGAGTCAA CGTAAAGATA AAAATGATTA TTATCACAGT TTTAAAACCT GATTTAGGAG
TTGACCTGGG ATAAGACCTA AGTCACAGGT TGAGATGATT ATTGAACAAG GTCAATGTAA
AGATAAAAGT GTTTATTATT ATAGTTTTAA AGTCTAACTA GGAGGTCAAC TCGGGTAAAG
TCCCTGTCAC AGGTTTGGTT AACCATTGAC CCAGGTCAAT ATAAAGATAA AAGTAGCTTG
AGTTACAAGT CGGGTTGACC ATTGATCTGA GTTAATGTAA GGATAAAAAT AGTTATTATC
ATAGTATTAA AACTCGACTA GGAGATCGAC CTGAGGTAAG ACCCATGTCA CGGGTCGAGA
TGGTCAACTT GTTTTGACCC AAGATAACGT AAGAGTAAGA ATAATTATTA TCAAAATTTT
AAAATCCGAC TCGAGGTTCA GCTTGAGTCA TGGGTTGGGA GGGTTAACGT AAGAATAAAA
ATGATTATTA TTATAATTTT AAAACTCAAT TTAAAGGTCG ATCCGGGGCA AGGCCTGAGT
TATGGGTTGA GATGGTCAGC TTGGGTTGAC TTAGGTCAAT ATATAGATAA AAATAGTTAT
TATCATAATT TTAAAACCTA ACTTGGGGGT CGACCTGGGG CAAGGTTTGA GTCTTAAGAT
GAGAGGGTCA ACCTAAGTTG ACAAAATATT TTGTTTTTTA AAAAAATCAA AACAATTTTG
TTTTATAAAA AAAAATAAAA AAAGTCAAAA GGATTTCTTA GCCTTGTTTT ATCTTGGGTC
GCAAGTCGAC CTGATTTTTT AACCGAGTAG GGTCGGATCA ATTGTTTCTT TATTTTTTCT
CAAACTTAGA TTAGTCCAAT TCTTGTGTCA GTCGGGTCTC ATCCGGTATC TACCAAACAC
GAAATAAAAT AAATTGTCTA GTTCAATTCA AAGCACACTA AATACAAAAA CAAAACAATT
ATTTGTCGAG TCCAATTAAT ATTGTCTAGC ATACCAAACA CTACCCTACC ACTCATAGCC
TTTGTGCAAA ATGTTGAGGG ACCTCGAACG TTTTATTTAT TGCATCACTT GTGCTAATAC
TGTTTTGTCA ATGATAAAAA AGATGGTTAT TTATGATCTA ATGATGAACA CTTCTATAAT
TCAAAACTGA AGTTAACTGA TATATAGTTT TACAAATATT TTGGACAACA TTTTTTAAAT
TTCAGGGATG AAAATCTTAA AGTTCAAAAG CCGTTTTGAT CTAGTTTCTG TTGGAATTCA
AATCACATAC CATGTGAACT CCCTCTTTTT TCCTCCATCC TTCTCTATGG TCACATTTTT
TTCATTAAGT TTTTTCATAT CTATTTTTAA GCTCATTTTC TGTACTTCCT CATTGTAGTT
ATGGTAATTT TCTTCCAATG CACACAGGAT ATTACCTTAC AAAAGTTTCA TCTAATCCTG
GAGCTGGTGA TGTGTTAATG GGATTCTTGG GACCTGTCAT TCTTTCTTTT GCCTTCTCAA
TGTTCAAGCA GCGAAAGGTA ACTTCTTTTC TCACAATCCA TGCCAACAGG TCGGGTTCAT
TTTGTATTAA GTGGATATGT TTCATCTGTG CTGTTACTCA GGAAGGACTA GGGGTTCCTT
TTATTTAATT GTAACTATTT GGTGTTGCCA ACCAAACTAT CTGGTGTTGC CAACCAAACT
ATCTGCTATA CATTATTTTT TTTCATTTGG TAATGGAAGA CGATGCGTTT GAAATTCAGA
GTAGAGAATA AGCAATGAAA AACCTCAAAA TATTTAGACC TTGCAAGAAT CTAGGAATCT
AGAATATTGC ATCTACCTTT ATATGAATTT ATCCTCAGTA GTATTCTATG TATGCTTTGT
CATTACATGG TTTGTTGTCC TCGATTCAGA ATAATAAAAA TTTGTCATCA GATGATGCTC
TTAACTTACT GGTCCTTTTG TCATGGCAAT GTGAGTTTAT ATACTCTGGT GTTTCTGCAG
CTTGTTAAGA GACATGCAGC TGAGATTTTC ACGTCCGTCA TTGTTGCAAC ACTATTTTCA
TTGTATTCAA CGGCTCTCGT GGGACGTCTA GTTGGGTTAG AACCAACGTT GACTGTATCC
ATTATTCCCA GATGTATAAC CGTGGCATTA GCCCTCAGCA TTGTGTCATT CTTTGAAGGT
ATAGTAATGT TCAGAAGCCA GCTGGAAGCA GGGAATTCCA TAAATGGGCA TCCACTAGTG
ATGCTAAAAT TAAATTTTGA CATATCAAAC ACCGTTATTC TGAGATGAAT TCATTTCCAA
TTTACTTAAC GACTTTGGTC AGTTAGAACA CAATGTACTG CAGATGATTG GTGTGACTCA
TTACCATTTT TAATCGAATT AATTTTACAT ATTGGTTGTA GGTGCCAATT CATCTCTCAC
AGCTGCTGTA GTTGTTGTAA CTGGTCTCAT TGGAGCAAAT TTTGTACAAG CAGTGCTCGA
TAAATTAAAT TTTCGTGATC CCATTGCTCG AGGAATAGCA ACTGCTTCCA GGTTTGTGGT
TTACCAAAGA CAAATCAGTA CGAACTCAAG CACTAAACAA TCTTTGCAAG CATAGACCCT
TCCCTTTCTT TAGTAAATAT AATGATGTGT CATATCAGCT ACAAGCAACT ATCTTTCTCA
TAGCTCACCT GTATAGTTTT CTTTCAGGGG ACTGCAACAA AAATGCATCT GGGACCAATA
CTTTGTACTA ACAAAATGCT TGCTGTTGGC CAGAAACTGG AAACTGAATC TCCTGGGGAA
CTTAAAACTG GTTAATAATT AAGCTATTTA ATATGTAGAA AATTCTGAAT GTCACTGAGA
TTCTTATTGC TGAATCTTAT AAAAATCAAA ATCATTTTAG AATTGGACTA GTCAATGAAA
GGAGATCTGT GTTTTTCTGT TTTGGTTGAA TATTTTAGTC attttGGtta TAGCATATAA
AAACAAAACC GTGGATTGAA AATAAGAAGA AAGATAGGAT GCTCGAACTT TGCTCACTGA
ATTGGACTTG ATGCACAATC TTGAGTTATG CTTTAAAAAC CTTACCCAAG TTTGTTTGTG
ACTTCGGTTA TAGTCGCAGC TTGTGATGCT CAGATAAGCT CAGTTTACCT GTTCTGTTCA
AGAGTGTACA ACCTGACATG CTCAGTCCTG AGTATTATAT TCATATCCTA CTTTAGTTTT
CCTGTGTGAA GATTTTGCAA AGTTTTGTAG GAACTGTGTC CCATTACTGC ATTTATGCTT
ATCCTTCTTA ATTGTTTCAG TGCTCATGGA CTGGGAACTG CAGCATTGTC AGCCAAGGAA
CCTGAGGCAC TTCCATTTTG TGCCATTGCT TATGCTCTCA CTGGTATATT TGGTTCATTG
TTTTGTTCAG TTCCTGCAGT TAGGCAAAGC TTACTTGCAA TAATTGGCTG AAAGATTGGG
CAGCTTTTGG TTATTGGTGG CCAGACACAC TTTTGATTGA TTATCCTTGT CCACCCACAG
CATGATTTTT AGCAAGTACA CAGTGCTAAT AGCCAACCTT GTATTATTGA TTACAGTCAA
TTATGTATAG AAGTTGACAT CATCCTTCTG CAAAAACTCC TCACCTCCTT TAAATTCCTT
TAAATTATGA ATAGTGAACT GATTGTCTTC TTGTTAGAAG GCGGTCAGCT GGCTGAAAAG
CAAAAGCTAA AGGTCAAAGG TAAATTATCT ATTCTACACC TTGAGAGGGT GGAAAATCCA
GTGGATACCA AAGAAGCCAC TGTTGCTCAG

The coding sequence for PtPlgg1 (SEQ ID NO:6) (GenBank Accession No. XM_0245597884 (LOC18096994), which is hereby incorporated by reference in its entirety) is as follows:

ATGGCTACTC CTTTAGTCGC TCATTCCGTT CAGCTCTGTC ATCACCACTC AAAACAATAT
TCTTTCAAAT CACAATCACG TGTCAATAGG GATATCGGTA CAAAAACACT ACTTGGTGTT
TACAATGGAT TCCACAATGT ATATAACCAA TCTTATTTTC ATAAGCCTTG GGCACCCATT
AGAGTTCTTG AGCCTAATTC AAGGTTCTTG CAAATGGGTC CTCAAGAAAC CTGCTCTAGT
CGAGGAATTT CCAAGAAATC TATGAGCTCA GAAGGCAGTA CCAGTACTAG CTCTTCATCT
ATTTCTCAAC AGGTGATTGG GATCTTGCAT TTGCTTGTTT CACTTGGGAT TATCCTTGCA
ATGGATAAGT TGTTGAAAAA GGCATTTGTG GCTGCTGCTA TCAAGTTTCC AAGTGCTCTG
TTTGGCATGT TCTGCATATT CTCAGTTTTA GTCATTCTTG ATATAACTAT TCCGGCTGCT
GCAACAAGCT TAATGAACTT CTTTCAGCCA GCACTGTTAT TCATTCAGAG ATGGCTTCCG
TTGTTCTACG TTCCATCATT GGTTGTTTTG CCTCTCTCTG TTAAAGATAT CCCTGCTGCA
TCAGGTGTCA AGATTTGCTT CATCACAGCT GGAGGGTGGT TGGCGTCACT TTGTGTGGCG
GGTTTTACAG CTATTGCTGT GAGAAAAATG GTGAAGACAG AAATGACTGA TGCTGAGCCT
ATGGCGAAAC CCTCTCCTTT TTCTCCATTG GAAATATGGG CTTGGAGTGG GGTCTTCCTT
GTATCATTTG TTGTCGCATT ATTATATAGA ACGGCATTAG GGACTGCAGC CAGAACATGC
CTTCCTTTCC TACTAGCGTC CACCGTGCTA GGCTATATGG TTGGTTCTGG GTTACCATCT
GGTGTTAAGA AGGTTTTCCA TCCCATTATT TGTTGTGCAC TATCTGCAGA TTTGGCAGCG
TTGGCCTTTG GGTTCCTTTC CCAATCTGGA CTCGATCCCG TTCTAGGATA TTACCTTACA
AAAGTTTCAT CTAATCCTGG AGCTGGTGAT GTGTTAATGG GATTCTTGGG ACCTGTCATT
CTTTCTTTTG CCTTCTCAAT GTTCAAGCAG CGAAAGCTTG TTAAGAGACA TGCAGCTGAG
ATTTTCACGT CCGTCATTGT TGCAACACTA TTTTCATTGT ATTCAACGGC TCTCGTGGGA
CGTCTAGTTG GGTTAGAACC AACGTTGACT GTATCCATTA TTCCCAGATG TATAACCGTG
GCATTAGGCC TCAGCATTGT GTCATTCTTT GAAGGTGCCA ATTCATCTCT CACAGCTGCT
GTAGTTGTTG TAACTGGTCT CATTGGAGCA AATTTTGTAC AAGCAGTGCT CGATAAATTA
AATTTTCGTG ATCCCATTGC TCGAGGAATA GCAACTGCTT CCAGTGCTCA TGGACTGGGA
ACTGCAGCAT TGTCAGCCAA GGAACCTGAG GCACTTCCAT TTTGTGCCAT TGCTTATGCT
CTCACTGGTA TATTTGGTTC ATTGTTTTGT TCAGTTCCTG CAGTTAGGCA AAGCTTACTT
GCAATAATTG GCTGA

The amino acid sequence of PtPlgg1 (SEQ ID NO:7) (GenBank Accession No. XP_024453652, which is hereby incorporated by reference in its entirety) is as follows:

MATPLVAHSV QLCHHHSKQY SFKSQSRVNR DIGTKTLLGV YNGFHNVYNQ SYFHKPWAPI
RVLEPNSRFL QMGPQETCSS RGISKKSMSS EGSTSTSSSS ISQQVIGILH LLVSLGIILA
MDKLLKKAEV AAAIKFPSAL FGMFCIFSVL VILDITIPAA ATSLMNFFQP ALLFIQRWLP
LFYVPSLVVL PLSVKDIPAA SGVKICFITA GGWLASLCVA GFTAIAVRKM VKTEMTDAEP
MAKPSPFSPL EIWAWSGVFL VSFVVALLYR TALGTAARTC LPFLLASTVL GYMVGSGLPS
GVKKVFHPII CCALSADLAA LAFGFLSQSG LDPVLGYYLT KVSSNPGAGD VLMGFLGPVI
LSFAFSMFKQ RKLVKRHAAE IFTSVIVATL FSLYSTALVG RLVGLEPTLT VSIIPRCITV
ALALSIVSFF EGANSSLTAA VVVVTGLIGA NFVQAVLDKL NFRDPIARGI ATASSAHGLG
TAALSAKEPE ALPFCATAYA LTGIFGSLFC SVPAVRQSLL AIIG

Additional exemplary Plgg1 sequences are described in Table 9 infra, and are intended to illustrate the various types of plant cells that may be transformed to form a transgenic plant cell of the present disclosure with an inhibitory polynucleotide targeting an endogenous glycolate transporter Plgg1. In some embodiments, the glycolate transporter Plgg1 is a Populus alba Plgg1 with a coding region of SEQ ID NO:38. In some embodiments, the glycolate transporter Plgg1 is a Populus tremula Plgg1 with a coding region of SEQ ID NO:39. In some embodiments, the glycolate transporter Plgg1 is a Populus euphratica Plgg1 with a coding region of SEQ ID NO:40. In some embodiments, the Plgg1 is a Populus tremula and a Populus tremuloides Plgg1. In some embodiments, the Plgg1 is a Populus tremula and a Populus alba Plgg1. In some embodiments, the glycolate transporter Plgg1 is from the genus Picea. In some embodiments, the glycolate transporter Plgg1 is from the species Picea abies with a coding region of SEQ ID NO:41. In some embodiments, the glycolate transporter Plgg1 is from the genus Pinus. In some embodiments, the glycolate transporter Plgg1 is from the species Pinus taeda with a coding region of SEQ ID NO:35.

The present disclosure also encompasses inhibitory polynucleotides that target endogenous glycolate transporter Plgg1 beyond the specific exemplary glycolate transporter Plgg1 sequences disclosed herein. Glycolate transporter Plgg1 sequences targeted by inhibitory polynucleotides of the transgenic cells of the present disclosure may include glycolate transporter Plgg1 molecules from other organisms that can be readily identified by methods known in the art such as through next generation sequencing and sequence identification based on sequence identity or homology at the nucleotide or the amino acid level. A person of ordinary skill in the art would readily appreciate that glycolate transporter Plgg1 across different genuses and species of plants may have some variation. Transgenic plant cells of the present disclosure may be made with an inhibitory polynucleotide targeting an endogenous glycolate transporter Plgg1 of any plant species.

In some embodiments, inhibitory polynucleotides targeting other endogenous or native chloroplast glycolate transporter Plgg1 coding sequences of other plant varieties and species comprise a glycolate transporter Plgg1 that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% sequence identity to any one of SEQ ID NO:6, SEQ ID NO:35, or SEQ ID NOs:38-41.

In some embodiments, the inhibitory polynucleotide targeting an endogenous glycolate transporter Plgg1 is an antisense or RNAi molecule comprising a nucleotide sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% sequence identity of any one of SEQ ID NO:6, SEQ ID NO:35, or SEQ ID NOs:38-41.

In some embodiments, the inhibitory polynucleotide targets a polynucleotide sequence coding for a Populus trichocarpa PtPlgg1 having an amino acid sequence of SEQ ID NO:7. In some embodiments, the targeted native chloroplast glycolate transporter Plgg1 has an amino acid sequence that is at least 80% identical to the amino acid sequence of Populus trichocarpa PtPlgg1 of SEQ ID NO:7. In some embodiments, the targeted glycolate transporter Plgg1 comprises an amino acid sequence that is at least 80%, 83%, 85%, 90%, 93%, 95%, 98%, 99%, or 100% identical to the amino acid sequence of Populus trichocarpa PtPlgg1 of SEQ ID NO:7.

In some embodiments, the inhibitory polynucleotide reduces the expression of the endogenous glycolate transporter Plgg1. As used herein, “reduces expression” refers to the amount or level of RNA or protein expressed in a cell being reduced compared to the amount or level of RNA or protein in a cell that does not have an inhibitory polynucleotide. Expression of RNA may be measured by methods known in the art. For example, RNA expression can be quantified by quantitative and semi-quantitative RT-PCR, RNA sequencing, microarrays, and Northern blots, as non-limiting examples. Protein expression can be quantified by mass spectrometry and western blots, as non-limiting examples.

Expression may be reduced at certain times in the cell or throughout the life of the cell. In some embodiments, expression of the endogenous glycolate transporter Plgg1 is about 10%-90% of the expression of endogenous glycolate transporter Plgg1 in a plant cell that is not transformed with an inhibitory polynucleotide targeting an endogenous glycolate transporter Plgg1, or any amount or range therein. In some embodiments expression of the endogenous glycolate transporter Plgg1 is about 10%, 20%, 30%, 40%, 50%, 60%, 70%, 80%, or 90% of the expression of endogenous glycolate transporter Plgg1 in a plant cell that is not transformed with an inhibitory polynucleotide targeting an endogenous glycolate transporter Plgg1. In some embodiments, expression of endogenous glycolate transporter Plgg1 is about 20% to 80% of the expression of endogenous Plgg1 in a plant cell that is not transformed, or any amount or range therein. In some embodiments, the expression of endogenous glycolate transporter Plgg1 is about 25% to 75% of the expression of endogenous glycolate transporter Plgg1 in a plant cell that is not transformed with an inhibitory polynucleotide targeting an endogenous glycolate transporter Plgg1, or any amount or range therein. In some embodiments, the expression of endogenous glycolate transporter Plgg1 is about 30% to 70% of the expression of endogenous glycolate transporter Plgg1 in a plant cell that is not transformed with an inhibitory polynucleotide targeting an endogenous glycolate transporter Plgg1, or any amount or range therein. In some embodiments, expression of endogenous glycolate transporter Plgg1 is about 30% to 60% of the expression of endogenous glycolate transporter Plgg1 in a plant cell that is not transformed with an inhibitory polynucleotide targeting an endogenous glycolate transporter Plgg1, or any amount or range therein. In some embodiments, expression of endogenous glycolate transporter Plgg1 is about 40% to 60% of the expression of endogenous glycolate transporter Plgg1 in a plant cell that is not transformed with an inhibitory polynucleotide targeting an endogenous glycolate transporter Plgg1, or any amount or range therein.

The expression or activity of an endogenous glycolate transporter Plgg1 can also be reduced by mutating the endogenous glycolate transporter Plgg1. Thus, in some embodiments, the transgenic cell may not have an inhibitory molecule targeting an endogenous glycolate transporter Plgg1, but has a mutation that inhibits normal expression of endogenous glycolate transporter Plgg1. The terms “mutation” or “genome edit” mean a human-induced change in the genetic sequence compared to a wild type sequence (i.e., a sequence that exists in nature). The mutation may be, without limitation, one or more nucleotide insertions, one or more nucleotide substitutions, one or more nucleotide deletions, or any combination thereof. In some embodiments, mutations are those that cause the gene coding sequence or promoter to not be expressed or to have a reduced level of expression, or those that reduce the activity of the encoded protein, such as from a conservative or other amino acid substitution or deletion, a splice junction mutation, or the introduction of a frameshift to the coding region that may lead to a premature stop codon.

In some embodiments, one or more mutations reduce expression of endogenous glycolate transporter Plgg1 by about 20% to 80%, or any amount or range therein, compared to endogenous glycolate transporter Plgg1 in a control. In some embodiments, one or more mutations reduce activity of the glycolate transporter Plgg1 protein by about 20% to 80%, or any amount or range therein compared to an un-mutated glycolate transporter Plgg1. In some embodiments, a mutation may be induced by treatment with a mutagenic agent. Any suitable mutagenic agent can be used for embodiments of the present disclosure. For example, mutagens creating point mutations, deletions, insertions, rearrangements, transversions, transitions, or any combination thereof may be used. Suitable radiation mutagens include, without limitation, ultraviolet light, x-rays, gamma rays, and fast neutrons. Suitable chemical mutagens include, but are not limited to, ethyl methanesulfonate (EMS), methylmethane sulfonate (MMS), N-ethyl-N-nitrosourea (ENU), triethylmelamine (TEM), N-methyl-N-nitrosourea (MNU), procarbazine, chlorambucil, cyclophosphamide, diethyl sulfate, acrylamide monomer, melphalan, nitrogen mustard, vincristine, dimethylnitrosamine, N-methyl-N′-nitro-nitrosoguanidine 25 (MNNG), nitrosoguanidine, 2-aminopurine, 7, 12 dimethyl-benz(a)anthracene (DMBA), ethylene oxide, hexamethylphosphoramide, bisulfan, diepoxyalkanes (diepoxyoctane (DEO), diepoxybutane (DEB), 2-methoxy-6-chloro-9[3-(ethyl-2-chloro-ethyl) aminopropylamino] acridine dihydrochloride (ICR-170), sodium azide, formaldehyde, or combinations thereof.

In some embodiments, a mutation may be induced by genome editing. Genome editing is a type of genetic engineering in which DNA is inserted, replaced, or removed, or any combination thereof, from a genome using artificially engineered nucleases or “molecular scissors.” The nucleases typically create double-stranded breaks (DSBs) at desired locations in the genome, and harness the cell's endogenous mechanisms to repair the induced break by processes of homology dependent repair (HDR) or nonhomologous end-joining (NHEJ). Any method of genome editing may be used in the embodiments of the present disclosure.

### Transgenic Plant Cell Cultures, Transgenic Plants, and Transgenic Trees

Another aspect of the present disclosure relates to a transgenic plant comprising the transgenic plant cell described herein.

A further aspect of the present disclosure relates to a transgenic plant cell culture produced by methods described herein below.

In the present disclosure, a transgenic plant cell may comprise a polynucleotide of any of the embodiments disclosed herein. A plant cell includes, without limitation, cells from plant tissue, including seeds, suspension cultures, embryos, meristematic regions, cotyledons, hypocotyls, callus tissue, leaves, roots, shoots, gametophytes, sporophytes, pollen, microspores, cotyledons, zygotic and somatic embryos, protoplasts, pollen, embryos, anthers, and the like.

Also provided herein are transgenic plant cell cultures, which comprise plant cells transformed according to the methods disclosed herein, and transgenic plants produced by the methods provided herein.

In one embodiment of the plant cell described above, the plant cell originates from a monocotyledonous or a dicotyledonous plant.

In another embodiment the plant cell originates from a genus selected from the group consisting of Hordeum, Sorghum, Saccharum, Zea, Setaria, Oryza, Triticum, Secale, Triticale, Malus, Brachypodium, Aegilops, Daucus, Beta, Eucalyptus, Nicotiana, Solanum, Coffea, Vitis, Erythrante, Genlisea, Cucumis, Marus, Arabidopsis, Crucihimalaya, Cardamine, Lepidium, Capsella, Olmarabidopsis, Arabis, Brassica, Eruca, Raphanus, Citrus, Jatropha, Populus, Medicago, Cicer, Cajanus, Phaseolus, Glycine, Gossypium, Astragalus, Lotus, Torenia, Allium, Spinacia, Helianthus, Populus, Eucalyptus, Pinus, Quercus, Pseudotsuga, Picea, Abies, Tsuga, Sequoia, Thuja, Chamaecyparis, or Ficus.

In some embodiments, the plant cell originates from a species selected from the group consisting of Hordeum vulgare, Hordeum bulbusom, Sorghum bicolor, Saccharum officinarium, Zea spp., including Zea mays, Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Allium tuberosum, Helianthus annuus, Helianthus tuberosus, and/or Spinacia oleracea.

In some embodiments, the transgenic plant cell is a transgenic tree plant. Suitable trees include, but are not limited to, plant trees of the genus Populus, Eucalyptus, Pinus, Quercus, Pseudotsuga, Picea, Abies, Tsuga, Sequoia, Thuja, Chamaecyparis, or Ficus. In some embodiments, the transgenic plant cell is a transgenic tree cell of a genus selected from the group consisting of Populus, Eucalyptus, Pinus, Quercus, Pseudotsuga, Picea, Abies, Tsuga, Sequoia, Thuja, Chamaecyparis, and Ficus. In some embodiments, the transgenic tree cell is from a tree of the genus Populus. In some embodiments, the transgenic tree cell is from a hybrid tree of Populus tremula x Populus alba.

Transgenic plants may be produced by methods described herein. In some embodiments, a transgenic plant comprises the transgenic plant cell. In some embodiments, the transgenic plant is a transgenic tree plant. Suitable trees include, but are not limited to, Populus, Eucalyptus, Pinus, Quercus, Pseudotsuga, Picea, and Ficus trees. In some embodiments, the transgenic tree is of the genus Populus, Eucalyptus, Pinus, Quercus, Pseudotsuga, Picea, or Ficus. In some embodiments, the transgenic tree is of the genus Populus. In some embodiments, the transgenic tree is a hybrid of Populus tremula x Populus alba.

The methods and compositions of the present disclosure can be used over a broad range of plant species. Such plants, include, but are not limited to Populus species (P. alba, P. alba x P. grandidentata, P. alba x P. tremula, P. alba x P. tremula var. glandulosa, P. alba x P. tremuloides, P. balsamifera, P. balsamifera subsp. trichocarpa, P. balsamifera subsp. trichocarpa x P. deltoides, P. ciliata, P. deltoides, P. euphratica, P. euramericana, P. kitakamiensis, P. lasiocarpa, P. laurifolia, P. maximowiczii, P. maximowiczii x P. balsamifera subsp. trichocarpa, P. nigra, P. sieboldii x P. grandidentata, P. suaveolens; P. szechuanica, P. tomentosa, P. tremula, P. tremula x P. tremuloides, P. tremuloides, P. wilsonii, P. canadensis, and P. yunnanensis).

Such plants, also include, but are not limited to, Eucalyptus species (E. alba, E. albens, E. amygdalina, E. aromaphloia, E. baileyana, E. balladoniensis, E. bicostata, E. botryoides, E. brachyandra, E. brassiana, E. brevistylis, E. brockwayi, E. camaldulensis, E. ceracea, E. cloeziana, E. coccifera, E. cordata, E. cornuta, E. corticosa, E. crebra, E. croajingolensis, E. curtisii, E. dalrympleana, E. deglupta, E. delegatensis, E. delicata, E. diversicolor, E. diversifolia, E. dives, E. dolichocarpa, E. dundasii, E. dunnii, E. elata, E. erythrocorys, E. erythrophloia, E. eudesmoides, E. falcata, E. gamophylla, E. glaucina, E. globulus, E. globulus subsp. bicostata, E. globulus subsp. globulus, E. gongylocarpa, E. grandis, E. grandis x urophylla, E. guilfoylei, E. gunnii, E. hallii, E. houseana, E. jacksonii, E. lansdowneana, E. latisinensis, E. leucophloia, E. leucoxylon, E. lockyeri, E. lucasii, E. maidenii, E. marginata, E. megacarpa, E. melliodora, E. michaeliana, E. microcorys, E. microtheca, E. muelleriana, E. nitens, E. nitida, E. obliqua, E. obtusiflora, E. occidentalis, E. optima, E. ovata, E. pachyphylla, E. pauciflora, E. pellita, E. perriniana, E. petiolaris, E. pilularis, E. piperita, E. platyphylla, E. polyanthemos, E. populnea, E. preissiana, E. pseudoglobulus, E. pulchella, E. radiata, E. radiata subsp. radiata, E. regnans, E. risdonii, E. robertsonii, E. rodwayi, E. rubida, E. rubiginosa, E. saligna, E. salmonophloia, E. scoparia, E. sieberi, E. spathulata, E. staeri, E. stoatei, E. tenuipes, E. tenuiramis, E. tereticornis, E. tetragona, E. tetrodonta, E. tindaliae, E. torquata, E. umbra, E. urophylla, E. vernicosa, E. viminalis, E. wandoo, E. wetarensis, E. willisii, E. willisii subsp. falciformis, E. willisii subsp. willisii, and E. woodwardii).

Such plants, also include, but are not limited to, conifers. Exemplary conifers include, without limitation, trees of the genus Pinus, including, for example, loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

The transgenic plants (and trees) obtained by the methods disclosed herein show enhanced biomass productivity and have increased carbon content compared to a non-transgenic plant or a non-transgenic tree of the same species. Transformation of plants and forest trees by the disclosed methods increases biomass productivity by 10-40% or more.

Increased biomass can be measured, for example, as an increase in plant height, plant total leaf area, plant fresh weight, plant dry weight, plant volume, plant stem, plant root, plant seed, lumber yield, or any combination thereof, as compared with control plants. In some embodiments, the transgenic plant has increased biomass accumulation compared to a plant of the same species not comprising the transgenic plant cell. In some embodiments, the transgenic plant has at least 5% to 100%, or any amount or range therein, or greater than 100% more biomass accumulation compared to a plant of the same species not comprising the transgenic plant cell. In some embodiments, the transgenic plant has at least 10% more biomass accumulation compared to a non-transgenic plant of the same species. In some embodiments, the transgenic plant has at least 20% more biomass accumulation compared to a non-transgenic plant of the same species. In some embodiments, the transgenic plant has at least 30% more biomass accumulation compared to a non-transgenic plant of the same species. In some embodiments, the transgenic plant has at least 40% more biomass accumulation compared to a non-transgenic plant of the same species. In some embodiments, the transgenic plant has more stem biomass compared to the stem biomass of a plant of the same species not comprising the transgenic plant cell. In some embodiments, the transgenic plant has at least 5%to 100%, or any amount or range therein, or greater than 100% more stem biomass accumulation compared to the stem biomass of a plant of the same species not comprising the transgenic plant cell. In some embodiments, the transgenic plant has more leaf biomass compared to the leaf biomass of a plant of the same species not comprising the transgenic plant cell. In some embodiments, the transgenic plant has at least 5% to 100%, or any amount or range therein, or greater than 100% more leaf biomass accumulation compared to the leaf biomass of a plant of the same species not comprising the transgenic plant cell. In some embodiments, the transgenic plant has more root biomass compared to the root biomass of a plant of the same species not comprising the transgenic plant cell. In some embodiments, the transgenic plant has at least 5% to 100%, or any amount or range therein, or greater than 100% more root biomass accumulation compared to the root biomass of a plant of the same species not comprising the transgenic plant cell.

Biomass may be measured at various time points, including, for example and without limitation, 3 months to several years after seed germination or planting, or any amount of time therein.

In some embodiments, the transgenic plant has more height compared to the height of a plant of the same species not comprising the transgenic plant cell. In some embodiments, the transgenic plant has at least 5% to 100%, or any amount or range therein, or greater than 100% more height compared to the height of a plant of the same species not comprising the transgenic plant cell. In some embodiments, the transgenic plant has more stem volume compared to the stem volume of a plant of the same species not comprising the transgenic plant cell. In some embodiments, the transgenic plant has at least 5% to 100%, or any amount or range therein, or greater than 100% more stem volume compared to the stem volume of a plant of the same species not comprising the transgenic plant cell.

### Polynucleotide Constructs

A further aspect of the present disclosure relates to a polynucleotide construct comprising an inhibitory polynucleotide comprising a polynucleotide sequence that targets an endogenous glycolate transporter Plgg1 present in a plant cell, wherein when expressed in the plant cell, the inhibitory polynucleotide reduces expression of the endogenous glycolate transporter Plgg1 by about 20% to 80% of expression of endogenous glycolate transporter Plgg1 in a plant cell where the inhibitory polynucleotide is not present.

This aspect of the present application can be carried out with any of the embodiments disclosed herein.

The polynucleotide sequences of the present disclosure may be present on one or more constructs such as a plasmid for use in transformation, or on linear fragments of DNA. In some embodiments, the polynucleotide construct comprises one or more of the polynucleotide sequences disclosed herein. In some embodiments, the polynucleotide construct comprises a first polynucleotide sequence encoding a glycolate dehydrogenase, and a first promoter operably linked to the first polynucleotide sequence. In some embodiments, the polynucleotide construct comprises a second polynucleotide sequence encoding a malate synthase, and a second promoter operably linked to the second polynucleotide sequence.

In some embodiments, the polynucleotide sequence that targets an endogenous glycolate transporter Plgg1 present in a plant cell comprises 20-1000 nucleotides of any one of SEQ ID NO:6, SEQ ID NO:35, or SEQ ID NOs:38-41. In some embodiments, the polynucleotide sequence that targets an endogenous glycolate transporter Plgg1 present in a plant cell comprises 20-1000 nucleotides comprising a nucleotide sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% sequence identity of any one of SEQ ID NO:6, SEQ ID NO:35, or SEQ ID NOs:38-41.

In some embodiments, the inhibitory polynucleotide comprises SEQ ID NO:8 and SEQ ID NO:9. In some embodiments, the inhibitory polynucleotide comprises SEQ ID NO:36 and SEQ ID NO:37.

In some embodiments, the glycolate dehydrogenase comprises a nucleotide sequence of any one of SEQ ID NO:1, 46, or 48. In some embodiments, the glycolate dehydrogenase comprises a nucleotide sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% sequence identity of any one of SEQ ID NO:1, 46, or 48. In some embodiments, the malate synthase comprises a nucleotide sequence of any one of SEQ ID NO: 3, or 42-45. In some embodiments, the malate synthase comprises a nucleotide sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% sequence identity of any one of SEQ ID NO:3, or 42-45.

In some embodiments, the polynucleotide construct includes left and right Agrobacterium T-DNA border sequences (Peralta and Ream, “T-DNA Border Sequences Required for Crown Gall Tumorigenesis,” Proc. Natl. Acad. Sci. 82:5112-5116 (1985), which is hereby incorporated by reference in its entirety). These border sequences allow the introduction of heterologous DNA located between the left and right T-DNA border sequences into a host cell when using Agrobacterium-mediated DNA transformation.

Standard cloning procedures known in the art can be used to prepare the polynucleotide construct and/or the vector, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety.

### Methods of Increasing Biomass Productivity in Plants

Another aspect of the present disclosure relates to a method for increasing biomass productivity in a plant. This method involves transforming plant cells with one or more polynucleotides comprising a first promoter operably linked to a polynucleotide sequence encoding a glycolate dehydrogenase, a second promoter operably linked to a polynucleotide sequence encoding a malate synthase, and an inhibitory polynucleotide targeting an endogenous glycolate transporter Plgg1, where expression of the endogenous glycolate transporter Plgg1 in the transformed plant cell is about 20% to 80% of expression of endogenous glycolate transporter Plgg1 in a plant cell that is not transformed with an inhibitory polynucleotide targeting an endogenous glycolate transporter Plgg1, wherein said transforming increases biomass productivity of the plant.

This aspect of the present disclosure can be carried out with any of the embodiments disclosed herein.

Also provided herein are transgenic plant cell cultures, which comprise plant cells transformed according to the methods disclosed herein, and transgenic plants produced by the methods provided herein. In some embodiments, the methods comprise a transgenic plant cell culture. Some embodiments may further comprise culturing the transgenic plant cells under conditions effective to obtain transgenic plants.

In some embodiments, the polynucleotide construct comprising the polynucleotide sequences of the present disclosure is introduced into a host cell. “Introduced” includes the incorporation of a nucleotide into a eukaryotic or prokaryotic cell, where the nucleotide may be incorporated into the genome of the cell, and includes reference to the transient provision of a nucleotide or protein to the cell. The term also may include a reference to stable or transient transformation methods, as well as sexually crossing. Thus, “introduced” in the context of inserting a polynucleotide fragment (e.g., a polynucleotide construct/expression construct) into a cell, means transfection, transformation, or transduction and includes the incorporation of a nucleotide fragment into a eukaryotic or prokaryotic cell where the nucleotide fragment is incorporated into the genome of the cell (e.g., chromosome, plasmid, plastid, or mitochondrial DNA), converted into an autonomous replicon, or transiently expressed.

Polynucleotide constructs and vectors can be introduced into cells via transformation. Transformation refers to both stable transformation and transient transformation. A transient transformation refers to the introduction of the polynucleotide construct into the plant cell of a host organism resulting in gene expression without genetically stable inheritance.

A stable transformation refers to the introduction of the polynucleotide construct into a genome of a host organism resulting in genetically stable inheritance. Once stably transformed, the nucleotide fragment is stably integrated in the genome of the host organism and any subsequent generation.

Selectable markers may be used to select for plants or plant cells that comprise a polynucleotide construct. Selection of transformed cells comprising the polynucleotide construct utilizes an antibiotic or other compound useful for selective growth as a supplement to the media. The compound to be used will be dictated by the selectable marker element present in the vector with which the host cell was transformed. The marker may encode biocide resistance, antibiotic resistance (e.g., kanamycin, Geneticin (G418), bleomycin, hygromycin, etc.), or herbicide resistance (e.g., glyphosate, glufosinate, etc.). Examples of selectable markers include, but are not limited to, a neo gene which codes for kanamycin resistance and can be selected for using kanamycin, G418, etc.; a bar gene which codes for bialaphos resistance; a mutant EPSP synthase gene which encodes glyphosate resistance; a nitrilase gene, which confers resistance to bromoxynil; a mutant acetolactate synthase gene (ALS), which confers imidazolinone or sulfonylurea resistance; and a methotrexate resistant DHFR gene. Multiple selectable markers are available that confer resistance to ampicillin, bleomycin, chloramphenicol, gentamycin, hygromycin, kanamycin, lincomycin, methotrexate, phosphinothricin, puromycin, spectinomycin, rifampicin, streptomycin and tetracycline, etc. Examples of selectable markers are described in, e.g., U.S. Pat. Nos. 5,550,318; 5,633,435; 5,780,708; and 6,118,047, which are hereby incorporated by reference in their entirety.

The methods disclosed herein comprise introducing the polynucleotide sequences into plant cells by transformation. In some embodiments the polynucleotides are introduced into the plant cells by methods such as Agrobacterium tumefaciens infection, electroporation, particle bombardment, or protoplast transfection to obtain transgenic plant cells.

A variation of Agrobacterium transformation uses vacuum infiltration in which whole plants are used (Senior, “Uses of Plant Gene Silencing,” Biotechnology and Genetic Engineering Reviews 15:79-119 (1998), which is hereby incorporated by reference in its entirety). In some embodiments, transformation involves fusion of protoplasts with other entities, cither minicells, cells, lysosomes, or other fusible lipid-surfaced bodies (Fraley et al., “Liposome-Mediated Delivery of Tobacco Mosaic Virus RNA into Tobacco Protoplasts: A Sensitive Assay for Monitoring Liposome-Protoplast Interactions,” Proc. Natl. Acad. Sci. USA 79:1859-63 (1982), which is hereby incorporated by reference in its entirety).

In some embodiments, transformation can be accomplished by electroporation (Fromm et al., “Expression of Genes Transferred into Monocot and Dicot Plant Cells by Electroporation,” Proc. Natl. Acad. Sci. USA 82:5824 (1985), which is hereby incorporated by reference in its entirety). In this technique, plant protoplasts are electroporated in the presence of plasmids containing the expression cassette. Electrical impulses of high field strength reversibly permeabilize biomembranes allowing the introduction of the plasmids. Electroporated plant protoplasts reform the cell wall, divide, and regenerate.

In some embodiments, transformation can be accomplished through PEG-mediated DNA transfer, microinjection, or vacuum infiltration to provide for stable or transient expression of the polynucleotide construct. Other methods of transformation include polyethylene-mediated plant transformation, micro-injection, physical abrasives, and laser beams (Senior, “Uses of Plant Gene Silencing,” Biotechnology and Genetic Engineering Reviews 15:79-119 (1998), which is hereby incorporated by reference in its entirety). In any of these methods, transformation can be enhanced by the use of a suitable microbe, such as a Rhizobia microbe, or an Agrobacterium, to facilitate DNA uptake by plant cells.

In some embodiments, transient or stable transformation of polynucleotide constructs described herein is performed using particle bombardment (also known as biolistic transformation). In some embodiments, particle bombardment involves propelling inert or biologically active particles at cells. This technique is disclosed, for example, in Klein et al., “High-Velocity Microprojectiles for Delivering Nucleic Acids Into Living Cells,” Nature 327:70-73 (1987), which is hereby incorporated by reference in its entirety, and is also known as biolistic transformation of the host cell, as disclosed in U.S. Pat. Nos. 4,945,050; 5,036,006; and 5,100,792, all to Sanford et al., and in Emerschad et al., “Somatic Embryogenesis and Plant Development from Immature Zygotic Embryos of Seedless Grapes (Vitis vinifera),” Plant Cell Reports 14:6-12 (1995), which are hereby incorporated by reference in their entirety. Generally, this procedure involves propelling inert or biologically active particles at the cells under conditions effective to penetrate the outer surface of the cell and to be incorporated within the interior thereof. When inert particles are utilized, the vector can be introduced into the cell by coating the particles with the vector containing the heterologous DNA. In some embodiments, the target cell can be surrounded by the vector so that the vector is carried into the cell by the wake of the particle. Biologically active particles (e.g., dried bacterial cells containing the vector and heterologous DNA) can also be propelled into cells.

In some embodiments, biolistic methods use gold or tungsten particles typically of 0.5 to 2 micrometers in size and coated with DNA, RNA, or ribonucleotide particles that has been precipitated onto the particles; the particles are discharged using a “gene gun” powered by a gas at high pressure (typically hundreds to thousands pounds per square inch) onto a plant held in an evacuated chamber. Some biolistic methods using equipment such as the Helios® gene gun (Bio-Rad Laboratories, Inc.) use lower pressures (in the hundreds pounds per square inch). Biologically active particles (e.g., dried bacterial cells containing the vector and heterologous DNA) can also be propelled into plant cells. Other variations of particle bombardment, now known or hereafter developed, can also be used.

Methods for transforming trees are known in the art, such as described in U.S. Pat. No. 6,518,485, which is hereby incorporated by reference in its entirety, which discloses biolistic transformation method of gymnosperm somatic embryos, and Li et al., “Simple, Rapid and Efficient Transformation of Genotype Nisqually-1: A Basic Tool for the First Sequenced Model Tree,” Scientific Reports 7:2638 (2017), which is hereby incorporated by reference in its entirety. Any method of transformation that results in efficient transformation of the host cell of choice is appropriate for practicing the methods of the present disclosure.

In some embodiments, the disclosed methods may further comprise culturing the transgenic plant cells under conditions, which stimulate plant growth to obtain transgenic plants. In some embodiments, the transgenic plants show enhanced biomass productivity and/or have increased carbon content compared to non-transgenic plants of the same species.

After transformation, transformed plant cells can be regenerated. Means for regeneration vary from species to species of plant, but generally a petri plate containing explants or a suspension of transformed protoplasts is first provided. Callus tissue is formed and transformation of callus tissue can be performed. Shoots may be induced from callus and subsequently rooted. Alternatively, embryo formation can be induced in the callus tissue. These embryos germinate as natural embryos to form plants. The culture media will generally contain various amino acids and hormones, such as auxin and cytokinins. Efficient regeneration will depend on the medium, genotype, and history of the culture.

Transformed cells may first be identified using a selection marker simultaneously introduced into the host cells along with the polynucleotide construct or vector as described herein. Suitable selection markers are described above. Cells or tissues are grown on a selection medium containing the appropriate antibiotic, whereby generally only those transformants expressing the antibiotic resistance marker continue to grow. Other types of markers are also suitable for inclusion in the vector, such as reporter genes as described above. The selection employed will depend on the target species; for certain target species, different antibiotics, herbicide, or biosynthesis selection markers may be preferred.

Plant cells and tissues selected by means of an inhibitory agent or other selection marker are then tested for the acquisition of the polynucleotide construct. In some embodiments, a transgenic cell comprises the heterologous polynucleotides. In some embodiments, the transgenic cell is a transgenic plant cell. As used herein, a transgenic cell or plant cell comprises within its genome a heterologous nucleotide introduced by a transformation step. The heterologous nucleotide is stably integrated within the genome such that the nucleotide is passed on to successive generations. As used herein, “genome”, as it applies to plant cells, encompasses not only chromosomal DNA found within the nucleus, but organelle DNA found within subcellular components (e.g., mitochondrial, plastid) of the cell. The heterologous nucleotide may be integrated into the genome alone or as part of a polynucleotide construct. In some embodiments, a transgenic plant is regenerated from the transgenic cell. In some embodiments, a transgenic plant comprises the transgenic plant cell.

### Methods of Increasing Photosynthetic Activity and Stomatal Conductance in Plants

A further aspect of the present disclosure is directed to a method for increasing photosynthetic activity and stomatal conductance under drought conditions in a plant. This method involves transforming plant cells with one or more polynucleotides comprising a first promoter operably linked to a polynucleotide sequence encoding a glycolate dehydrogenase, a second promoter operably linked to a polynucleotide sequence encoding a malate synthase, and an inhibitory polynucleotide targeting an endogenous glycolate transporter Plgg1, wherein expression of the endogenous glycolate transporter Plgg1 in the transgenic plant cell is about 20% to 80% of expression of endogenous glycolate transporter Plgg1 in a plant cell that is not transformed with an inhibitory polynucleotide targeting an endogenous glycolate transporter Plgg1. The method further involves regenerating a plant from the transformed plant cell, and subjecting the plant to drought conditions. Said transforming increases photosynthetic activity and stomatal conductance under drought conditions in the regenerated plant.

This aspect of the present disclosure can be carried out with any of the embodiments disclosed herein.

As used herein, “drought” refers to a decrease in water availability to a plant that, especially when prolonged, can cause damage to the plant or prevent its successful growth (e.g., limiting plant growth or seed yield). Drought tolerance is a trait of a plant to survive under drought conditions over prolonged periods of time without exhibiting substantial physiological or physical deterioration. In some embodiments, drought can be estimated by soil moisture levels. In some embodiments, a soil moisture level >9.5 indicates no drought, a soil moisture level of 7.5-9.49 indicates mild drought, a soil moisture level of 5.5-7.49 indicates moderate or medium drought, and a soil moisture level <5.49 indicates severe drought.

Photosynthetic activity can be estimated, for example, by measuring maximum rate of photosynthetic electron transport (Jₘₐₓ) and/or maximum carbon assimilation rate (Aₘₐₓ). Photosynthetic activity can decrease during drought conditions, especially severe drought conditions.

Stomatal conductance estimates the rate of gas exchange and transpiration or water loss through the leaf stomata, and is a measure of the degree of stomatal opening. Stomatal conductance reflects the physical resistance of the movement of gases between air and the interior of the leaf, and thus indirectly reflects the state of photosynthetic activity. Stomatal conductance can be a sensitive indicator of stress. During drought conditions, stoma in leaves may close to reduce water loss, which will lead to a drastic decrease in stomatal conductance

Water use efficiency (WUE) is a metric that describes the potential water cost of carbon assimilation. WUE is measured as the ratio of net carbon assimilation to stomatal conductance to water vapor. Basically it reflects the ratio of water used for photosynthetic activity to water lost through transpiration. Under normal growth conditions where water supply is sufficient, higher WUE generally translates to higher photosynthetic activity. In drought conditions, stomata close to prevent water loss through transpiration resulting in WUE increases. The more severe the drought the higher the WUE. Some plants that cope well under the drought condition maintain some degree of stomatal conductance and photosynthetic activity. The rate of increase in WUE in those plants under drought conditions is smaller compared to that of plants that do not tolerate drought.

In some embodiments, the transgenic plant has increased stomatal conductance compared to a plant of the same species not comprising the transgenic plant cell during moderate or severe drought. In some embodiments the transgenic plant has at least 5% to 50%, or any amount or range therein, or more than 50% more stomatal conductance compared to a plant of the same species not comprising the transgenic plant cell during moderate or severe drought. In some embodiments, the transgenic plant has increased photosynthetic activity compared to a plant of the same species not comprising the transgenic plant cell during moderate or severe drought. In some embodiments the transgenic plant has at least 5% to 50%, or any amount or range therein, or more than 50% more photosynthetic activity compared to a plant of the same species not comprising the transgenic plant cell during moderate or severe drought.

The following examples are intended to exemplify the embodiments of the disclosure but are by no means intended to limit the scope thereof.

### EXAMPLES

### Example 1—RNAi Construct for Populus Plgg1

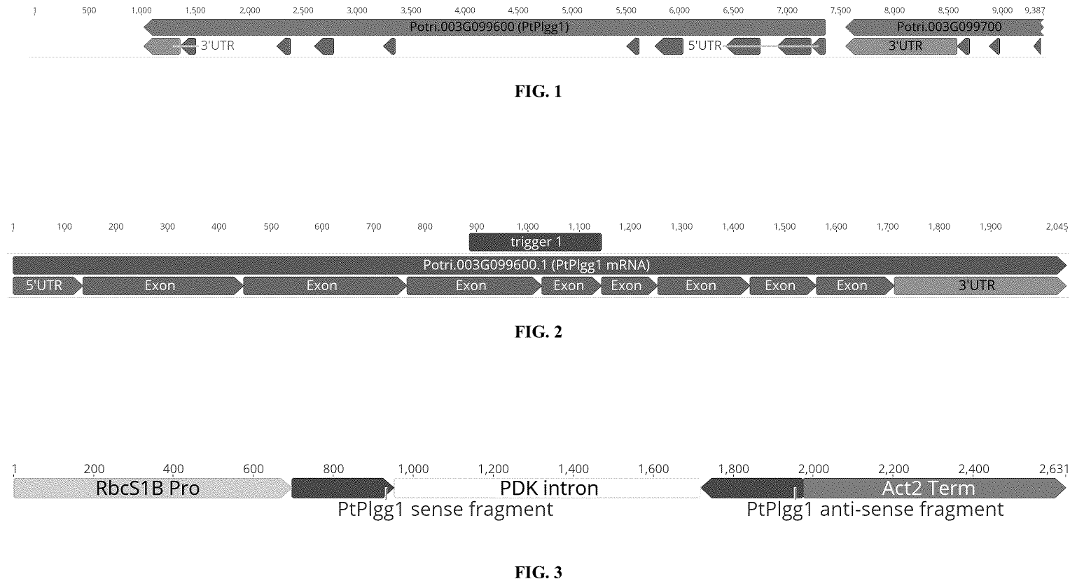

The genomic Populus trichocarpa PtPlgg1 sequence, is a 6.3 kb gene with 5 exons, large introns, a 136 bp 5′UTR and a 334 bp 3′UTR (SEQ ID NO:5). Immediately upstream of PtPlgg1 (185 bp) is the 3′UTR of neighboring Potri.003G099700 (FIG. 1).

The RNAi region selected for intron-containing hairpin RNAi (ihRNAi) of PtPlgg1 was a 256 nt region from 750-1,006 nucleotides of the coding sequence. These numbers correspond to nucleotides 886-1,142 of the transcript sequence (FIG. 2) and 750-1006 nucleotides measured from the start codon. The sequence used for the Populus PtPlgg1 RNAi construct in the sense direction (FIG. 3) is SEQ ID NO:8, as follows:

GGAAATATGG GCTTGGAGTG GGGTCTTCCT TGTATCATTT GTTGTCGCAT TATTATATAG
AACGGCATTA GGGACTGCAG CCAGAACATG CCTTCCTTTC CTACTAGCGT CCACCGTGCT
AGGCTATATG GTTGGTTCTG GGTTACCATC TGGTGTTAAG AAGGTTTTCC ATCCCATTAT
TTGTTGTGCA CTATCTGCAG ATTTGGCAGC GTTGGCCTTT GGGTTCCTTT CCCAATCTGG
ACTCGATCCC GTTCTAG

The sequence used for the Populus PtPlgg1 RNAi construct in the antisense direction (FIG. 3) is SEQ ID NO:9, as follows:

CTAGAACGGG ATCGAGTCCA GATTGGGAAA GGAACCCAAA GGCCAACGCT GCCAAATCTG
CAGATAGTGC ACAACAAATA ATGGGATGGA AAACCTTCTT AACACCAGAT GGTAACCCAG
AACCAACCAT ATAGCCTAGC ACGGTGGACG CTAGTAGGAA AGGAAGGCAT GTTCTGGCTG
CAGTCCCTAA TGCCGTTCTA TATAATAATG CGACAACAAA TGATACAAGG AAGACCCCAC
TCCAAGCCCA TATTTCC

This sequence was selected by blasting various regions of the Populus alba PaPlgg1 transcript to the Populus trichocarpa genome and selecting regions with a low number of “hits” elsewhere in the genome in an attempt to minimize off-target siRNAs. No attempt was made to estimate and select based on siRNA efficiency, or to map the off-target potentials of individual 21-mer siRNA sequences. The coding sequence of Populus alba Plgg1 (SEQ ID NO:38), one of the targets of RNAi, was greater than 98% identical at the nucleotide level to the coding sequence of Populus trichocarpa PtPlgg1 (Potri.003G099600) (SEQ ID NO:6). Similarly, the coding sequence of Populus tremula Plgg1 (Potra000595g04516) (SEQ ID NO:39) was greater than 98% identical at the nucleotide level to the coding sequence of PtPlgg1 (SEQ ID NO:6). An alignment between Plgg1 coding sequences and the area selected for the RNAi sense polynucleotide (SEQ ID NO:8) is shown in FIG. 4. Included in the alignment are Plgg1 sequences from both parents of the Poplar variety INRA 717-1B4, which is a hybrid between Populus tremula and Populus alba, and the Populus trichocarpa PtPlgg1. As shown below in Examples 3-11, the RNAi construct was effective at reducing expression of endogenous Plgg1 genes in a hybrid Populus tremula x Populus alba tree species and would be expected to be effective in other Populus species and hybrid species as well.

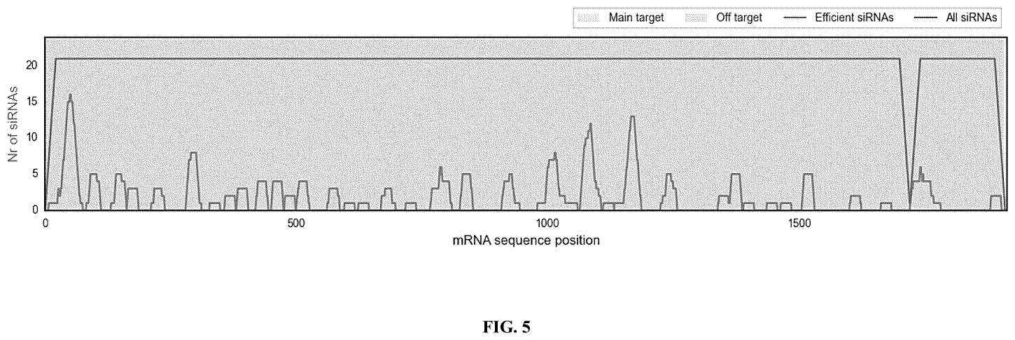

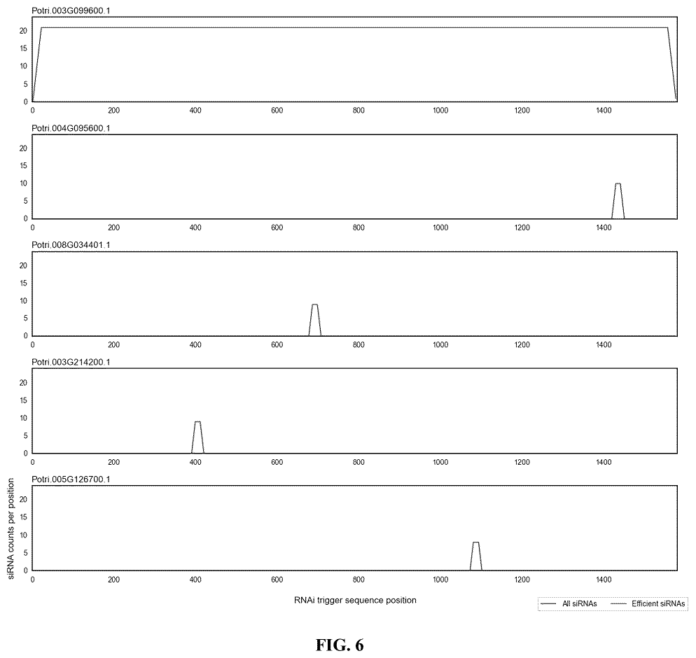

SiFi is a software disclosure for RNAi design and off-target prediction, which allows users to upload their own transcript database for off-target prediction (Luck et al., “siRNA-Finder (si-Fi) Software for RNAi-Target Design and Off-Target Prediction,” Front. Plant Sci. 10:1023 (2019), which is hereby incorporated by reference in its entirety). SiFi can be run in RNAi Design mode or Off-Target Prediction mode. There are also options to change siRNA size, alter the 5′ terminal nucleotide rule, strand selection, end stability difference with option to change difference, target site accessibility threshold with option to change threshold, and accessibility calculation window. The default settings used for this analysis are 21mers with (1 mismatch tolerated for design, and 3 for off-target prediction), 5′ terminal nucleotide rule on, strand selection on, end stability difference set to 1.00, target site accessibility threshold set to 0.10, and accessibility calculation window set to 8. RNAi design output on the PtPlgg1 mRNA sequence (FIG. 5) shows zones of highly efficient siRNAs, including a zone from 1000-1200 bp, a dense region around 500 bp, and confirmation that the beginning of the sequence has an adequate number of active siRNAs. Off-target prediction (presented in FIG. 6) shows regions to avoid right at approximately 1430 bp, 700 bp, 400 bp, and 1100 bp measured from the start codon.

In light of the data generated by siFi, two other promising trigger regions include the first 250 bp of SEQ ID NO:6, which presents as a cluster of efficient siRNAs with low off-target potential, and the region 400-650 bp of SEQ ID NO:6, which includes many potential siRNAs and low off-target potential. RNAi constructs using these sequences can be made in a similar fashion to the construct shown in FIG. 3, with a sense and antisense fragment of the chosen Plgg1 sequence. An intronic or other sequence may be used to separate the sense and antisense sequences. The sequence used in the PtPlgg1 construct with SEQ ID NO:8 and SEQ ID NO:9 was an intron of the Flaveria trinervia Pyruvate Orthophosphate Dikinase (“PDK”) (GenBank Accession No. X79095.1, which is hereby incorporated by reference in its entirety).

### Example 2—Tree Culture, Growth Conditions, Construction of Vectors and Genetic Transformation

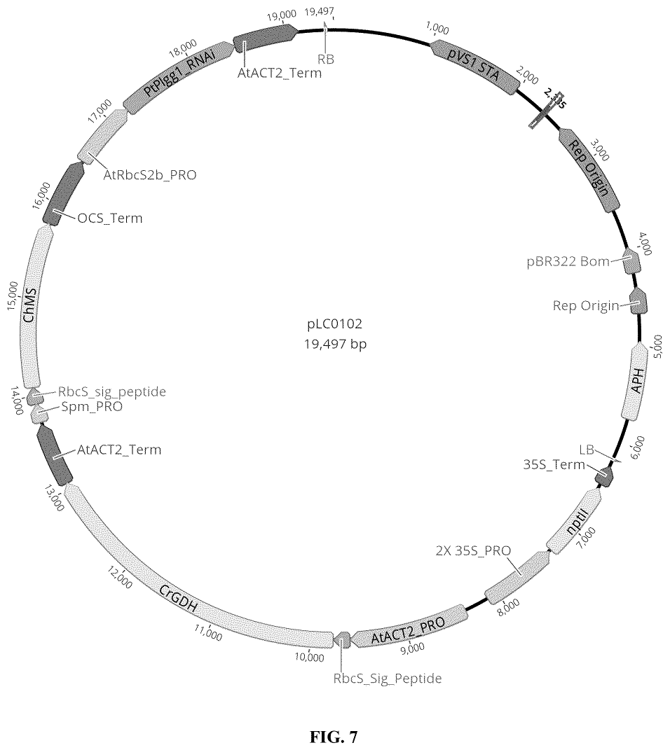

Construct pLC0102 comprising a gene encoding glycolate dehydrogenase from Chlamydomonas reinhardtii (SEQ ID NO:2) operably linked to the Arabidopis thaliana Actin 2 (“Act2”) promoter and terminator (GenBank Accession No. U41998.1, which is hereby incorporated by reference in its entirety), and the Arabidopis thaliana Rubisco Small Subunit (“AtRbcS”) signal peptide (GenBank Accession No. AY097366.1, which is hereby incorporated by reference in its entirety); a gene encoding malate synthase from Cucurbita sp. Kurokawa Amakuri Nankin (SEQ ID NO:4) operably linked to the Zea maize Suppressor Mutator (“Spm”) promoter (GenBank Accession No. M25427.1, which is hereby incorporated by reference in its entirety), the AtRbcS signal peptide, and the Agrobacterium tumifaciens Octopine Synthase (“OCS”) terminator (GenBank Accession No. CP033030.1, which is hereby incorporated by reference in its entirety); and an RNAi polynucleotide sequence (SEQ ID NO:8 and SEQ ID NO:9) targeting an endogenous glycolate/glycerate transporter Plgg1 separated by a PDK intron and operably linked to the Arabidopsis thaliana Rubisco Small Subunit 2 (AtRbcs2b) promoter (GenBank Accession No. X14564.1, which is hereby incorporated by reference in its entirety), and the Act2 terminator were transformed into Agrobacterium tumefaciens. The construct pLC0102 is shown in FIG. 7 and the Plgg1 RNAi cassette is shown in FIG. 3. FIG. 8 illustrates a construct with GDH and MS, which is useful for addition of other Plgg1 RNAi cassettes.

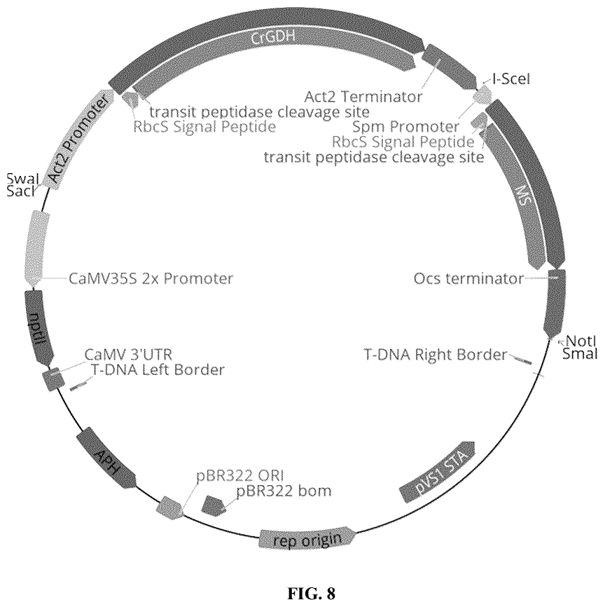

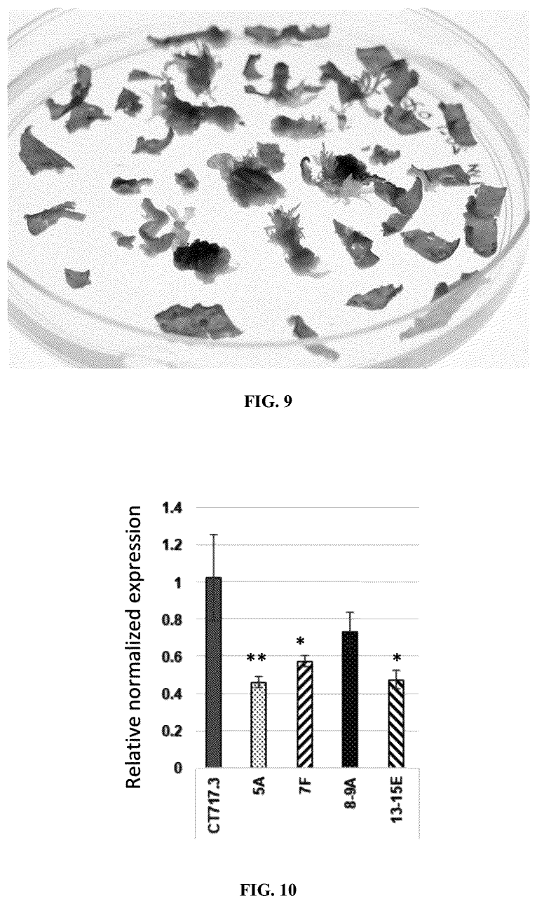

Recombinant Agrobacterium tumefaciens colonies transformed with pLC0102 were cultured for 24 hours on an orbital shaker. Leaves of the hybrid Populus tremula x Populus alba, clone INRA 717-1B4 were collected and used to grow stem explants. Stem explants were swirled in the Agrobacterium suspensions for 1 hour, and then co-cultured on agar medium for 48 hours at 24° C. in darkness. The explants were washed four times in 50 ml centrifuge tubes with deionized water at 125 rpm at 24° C., transferred to growth media with antibiotics for 21 days at 24° C. in darkness, and then transferred to standard light conditions 16 h light/8 h dark to recover green calli. The green calli were transferred to agar medium containing antibiotics for generation and selection of transformed shoots as shown in FIG. 9. The regenerated shoots were excised when they were approximately 1 cm long and transferred to agar medium supplemented with antibiotics for rooting. After 5-8 weeks, the rooted and elongated shoots were propagated on the same medium. Rooted LC0102 transgenic plants were transferred out of Magenta boxes in tissue culture into soil pots, acclimated, and grown under inverted diurnal cycle. A total of 41 stable pLC0102 TO transgenic events were evaluated and further propagated.

Eight weeks post potting, T0 plants went through a vegetative propagation cycle using a stem cutting method frequently used in horticulture practice. Briefly, the apical meristem was removed to release the apical dominance to stimulate the formation of axillary buds to produce branches. The axillary branches were cut and inserted into rockwool cubes for support to produce roots in hydroponic conditions. To maintain the genetic lineage of transgenic lines, cuttings from the T0 plant were named C1 plants with C1.1 referring to apical cutting and C1.2 and so on referring to axillary cuttings. C1 cuttings normally established root systems after approximately two weeks in hydroponic conditions and would then be transferred to soil pots for C1 plant evaluations. Multi branches or cuttings produced from one T0 mother plant are sister C1 plants called ramets, serving as biological replicates for C1 evaluation.

### Example 3—Transgenic Plant Phenotypes and Gene Expression Analysis

T0 leaf tissue samples were collected from transgenic trees as they were transferred out of Magenta boxes for the evaluation of gene expression. C1 leaf tissue samples were collected from 8 week old transgenic plants grown in soil pots in a controlled environment. Total RNA was extracted from leaf tissue samples using the NucleoSpin RNA Plants and Fungi extraction kit (Takara Bio Inc., Kusatsu, Japan). Quantitation of genomic DNA and total RNA was performed on a Nanodrop 1000 spectrophotometer (Thermo Fisher Scientific, Waltham, MA) following the manufacturer's protocol.

cDNA was generated from T0 RNA samples, and singleplex qRT-PCR analysis was performed to quantify Plgg1 reduction in the transgenic plants using the SYBR green RT kit from (Qiagen, Hiden, Germany). Briefly, a 20 μl RT-PCR reaction was assembled with 10 μl 2× SYBR Green RT-PCR Master Mix, 0.2 μl QN SYBR Green RT-Mix, 1 μl of 20× gene specific primer mix, 0.2 μl QN ROX dye, 1 μl 100 n g/μl template RNA, and 7.6 μl RNase-free ddH2O. The reaction wells were then placed in a Chai PCR machine (Chai, Santa Clara, CA) for PCR amplification with the following cycling program: Step 1, 50° C., 10 minutes; Step 2, 95° C., 2 minutes; Step 3, 95° C., 5 seconds; Step 4, 60° C., 10 seconds, repeating Step 3 to Step 4 for 40 cycles. Data acquisition and analysis was performed with the Chai machine. For each gene of interest, two RT-PCR reactions are prepared in parallel, one with the primer set to amplify the gene of interest, the other with the primer set to amplify the housekeeping gene ACT2. The level of gene expression was reflected by the Cq value generated from the RT-PCR reaction. Higher Cq value indicates lower level of expression.

Primers used for analyzing gene expression using the QuantiNova SYBR Green qRT-PCR kit are shown in Table 1.

TABLE 1
Primers Used for Gene Expression Analysis
Using SYBR Green qRT-PCR

| Gene Target | SEQ ID NO: | Sequence (5′ to 3′) |
|---|---|---|
| Plgg1 | 10 | CAGGTGTCAAGATTTGCTTCATC |
| | 11 | ACTCCAAGCCCACATTTCC |
| PtACT2 | 12 | ACTGAGACCATCACCAGAATC |
| | 13 | CTCAACCCTAAGGCTAACAGAG |
| ChMS | 14 | CCACATAAGGTATTCGATGGAATGC |
| | 15 | GACTGATGCACATGTCCACTC |
| CrGDH | 16 | CACATCAAGTTCCTGAAGCCCTC |
| | 17 | ATCTCGCGGTACACGGAGATG |
| nptII | 18 | CCTGCCGAGAAAGTATCCATC |
| | 19 | CATCCTGATCGACAAGACCG |

The expression analysis of transgenes via qRT-PCR was performed with T0 RNA samples using a Chai PCR machine (Chai, Santa Clara, CA). The expression level of the housekeeping gene ACT2 was similar among transgenic events and non-transgenic controls, whereas the expression level of transgenes and Plgg1 varied among different transgenic events. Expression data generated using SYBR Green assay demonstrated that 39 of the 41 transgenic events expressed all four genes in the pLC0102 construct. Table 2 shows the results of the relative gene expression level for transgene nptII, ChMS, and CrGDH as determined by the Cq value using SYBR green qRT-PCR assay. Non-transgenic controls CT717-1 and CT717-2 showed no expression of any of the transgenes, as expected, since they do not contain any of the transgene expression cassettes. Transgenic events 8-9, 8-9D, 16-20, and 16-20B showed no expression of transgenes, indicating they were transformation escapes (Table 2).

The 37 independent true transgenic events all showed varied Cq values indicating varied expression levels. As the primer efficiency for each gene is different, the Cq value of transgene A is only useful for comparison of relative expression level of transgene A among the transgenic events. Cq values are not useful for cross comparison of different transgenes in this case. Transgene expression level is summarized in Table 2 as 5 categories, with “+” indicating the lowest expression group and “+++++” indicating the highest expression group among the 37 transgenic events.

The expression level of the endogenous Plgg1 in transgenic events and non-transgenic control plants was also analyzed with T0 RNA samples using a Chai PCR machine (Chai, Santa Clara, CA). This analysis reveals the effectiveness of RNAi design in LC0102 construct. In this analysis, the expression level of Plgg1 in non-transgenic control plants is designated as 100% or 1.0-fold. Transgenic events harboring the RNAi design construct express RNAi at various levels and subsequently have various degrees of reduction effect on the expression of endogenous Plgg1.

Table 2 summarizes the relative expression level of Plgg1 in various transgenic events. Relative expression of Plgg1 is indicated with + symbols: +, less than 30%; ++, 30% to 60%; +++: 60% to 90%; ++++, 90% to 150%; +++++, greater than 150%. In the lowest Plgg1 expression group (“+” relative expression level in Table 2), events LC0102.2C and 2G show that the level of Plgg1 expression is approximately 10% of that of the non-transgenic control, reflecting a strong RNAi inhibition.

Analysis of plant morphology and the corresponding level of Plgg1 expression data generated using the Chai platform is shown in Table 2. It was found that transgenic plants with high reduction of Plgg1 with the lowest level of expression (“+”, <30% of the Plgg1 expression of control plants, such as LC0102.2C, 2G, 2A, 3C, 5F, 5E, 3H, and 13-15B) were more likely to show abnormal plant morphologies compared to controls. Transgenic events with normal or abnormal plant morphologies such as dwarfism are indicated in Table 2. Controls, including non-transgenic plants CT717-1 and CT717-2, and escapes from plant transformation with no expression of transgenes (LC0102.8-9, 8-9D, 16-20, and 16-20B) all showed normal plant morphologies. It was determined that most transgenic plants had normal morphologies with Plgg1 expression of at least 30% that of control plants. Further reduction of Plgg1 expression greater than about 30% (i.e., Plgg1 expression only 0-20% of Plgg1 expression in control plants) would likely render plants with abnormal morphologies.

Quantitative measurement of Plgg1 expression in transgenic events showing enhanced growth. As described in Example 2, vegetative propagation of T0 events was performed using stem cuttings to generate C1 plants. Total RNA prepared from leaf samples of selected groups of 8-week old C1 plants (transgenic events LC0102.13-15E, 5A, 7F, and 8-9A, and non-transgenic plants CT717.3) was analyzed for Plgg1 expression using a CFX Opus 96 Real Time PCR system (BioRad, Hercules, CA) in a multiplex assay with probe primers. Relative Plgg1 expression level was determined using the comparative CT method or ΔΔCT method with ACT2 as the reference gene (Schmittgen and Livak, “Analyzing Real-Time PCR Data by the Comparative CT Method,” Nature Protocols 3:1101-1108 (2008), which is hereby incorporated by reference in its entirety). FIG. 10 shows the expression level of Plgg1 in these plants. Three of the four transgenic events show significant differences in the level of Plgg1 expression compared to that of the non-transgenic control CT717.3, for which the expression level was designated as 1.0. In event 13-15E, 5A, and 7F, the average level of Plgg1 expression was reduced to 0.47, 0.46, and 0.57 (or 47%, 46%, and 57%), respectively, compared to the control. The expression level of Plgg1 in event 8-9A was also reduced (average value 0.73, or 73% of the control levels) but was not statistically significant.

TABLE 2
Plant Phenotypes and Relative Gene Expression in Transgenic Events

| | | | | | | Plgg1 | |
|---|---|---|---|---|---|---|---|
| | | | | | | qRT- | |
| | Relative Expression | | | | Plgg1 Fold | Repeats | |
| Event Name | nptll | ChMS | CrGDH | Plgg1 | Expression | PCR | T0 phenotype |
| LC0102.2C | ++++ | +++++ | +++ | + | 0.11 | 2 | Dwarf |
| LC0102.2G | +++++ | +++++ | ++ | + | 0.11 | 1 | >10 leaves fell |
| | | | | | | | off? |
| LC0102.2A | +++ | ++++ | ++ | + | 0.15 | 2 | Double tree |
| LC0102.3C | +++ | ++++ | ++ | + | 0.16 | 1 | Branches |
| LC0102.5F | +++ | ++++ | +++ | + | 0.18 | 2 | Branches |
| LC0102.21- | +++ | +++ | +++ | + | 0.19 | 2 | Normal |
| 25A | | | | | | | |
| LC0102.5E | ++++ | +++++ | +++ | + | 0.19 | 1 | Branches |
| LC0102.3H | +++ | ++++ | ++ | + | 0.22 | 2 | Snaky |
| LC0102.13- | ++ | ++++ | + | + | 0.23 | 2 | Forest |
| 15 B | | | | | | | |
| LC0102.2H | ++++ | ++++ | +++ | ++ | 0.31 | 1 | Branches |
| LC0102.5A | ++++ | +++++ | ++ | ++ | 0.32 | 2 | Normal |
| LC0102.21- | ++++ | +++++ | +++ | ++ | 0.33 | 1 | Normal |
| 25E | | | | | | | |
| LC0102.68 | +++ | ++++ | +++ | ++ | 0.36 | 2 | Normal |
| LC0102.5C | +++++ | +++++ | ++++ | ++ | 0.37 | 1 | Branches |
| LC0102.6 | ++ | ++ | ++ | ++ | 0.40 | 2 | Branches |
| LC01022 | +++ | +++ | +++ | ++ | 0.58 | 3 | Normal |
| LC0102.1 | +++++ | +++ | +++ | ++ | 0.60 | 2 | Normal |
| LC0102.3 | ++++ | +++ | ++++ | ++ | 0.60 | 2 | Branches |
| LC0102.5 | +++ | ++ | +++ | +++ | 0.67 | 3 | Normal |
| LC0102.4G | +++ | +++++ | ++ | +++ | 0.68 | 1 | Normal |
| LC0102.2F | ++ | +++++ | + | +++ | 0.7 | 1 | Moldy |
| LC0102.1B | +++ | ++++ | + | +++ | 0.79 | 2 | Normal |
| LC0102.1C | +++++ | +++ | ++++ | +++ | 0.85 | 1 | Normal |
| LC0102.13- | ++ | +++ | +++ | ++++ | 0.95 | 1 | Drought |
| 15 | | | | | | | induced |
| | | | | | | | abnormality |
| LC0102.4 | +++ | ++ | ++ | ++++ | 0.96 | 2 | Normal |
| LC0102.16- | − | − | − | ++++ | 1.00 | 2 | Branches |
| 20B | | | | | | | |
| CT717-1. | − | − | − | ++++ | 1 | 5 | Normal |
| LC0102.4A | +++ | ++++ | + | ++++ | 1.13 | 5 | Normal |
| LC0102.7 | ++++ | ++ | + | ++++ | 1.17 | 1 | Normal |
| LC0102.4B | ++++ | +++++ | ++ | ++++ | 1.22 | 2 | Branches |
| LC0102.8-9A | +++ | ++++ | +++ | ++++ | 1.24 | 1 | Normal |
| CT717-2 | − | − | − | ++++ | 1.28 | 1 | Normal |
| LC0102.3F | +++ | +++++ | +++ | ++++ | 1.38 | 3 | NA |
| LC0102.3B | +++ | ++++ | + | ++++ | 1.48 | 1 | Forest |
| LC0102.3D | ++ | +++++ | +++ | ++++ | 1.51 | 1 | Normal |
| LC0102.1A | +++++ | ++++ | +++++ | +++++ | 1.59 | 1 | Normal |
| LC0102.6D | ++++ | +++++ | ++++ | +++++ | 1.6 | 1 | Normal |
| LC0102.1D | ++++ | +++++ | +++ | +++++ | 1.93 | 1 | Normal |
| LC0102.16- | − | − | − | +++++ | 2.01 | 1 | Branches |
| 20 | | | | | | | |
| LC0102.7F | +++ | ++++ | ++ | +++++ | 2.6 | 1 | Normal |
| LC0102.8-9 | − | − | − | +++++ | 2.69 | 1 | Normal |
| LC0102.13- | +++ | ++++ | +++ | +++++ | 2.99 | 1 | Normal |
| 15E | | | | | | | |
| LC0102.8-9D | − | − | − | +++++ | 3.36 | 1 | Branches |

### Example 4—Multiplex Real-Time Gene Expression Analysis

Multiplex qRT-PCR analysis with technical duplicates in each event using CFX Opus 96 machine (Bio-Rad) was used for gene expression analysis of Plgg1, CrGDH, and ChMS. The 20 μL reaction contains 200 ng total RNA, 1× custom made One-Step RT-qPCR Master Mix with lower amount of DTT (Launchworks), primers, and probes (Table 3). qRT-PCR was performed with the following conditions: 53° C., 10 mins, reverse transcription; 95° C., 2 mins, initial denaturation; 40 cycles amplification (15 secs 95° C. denaturation; 30 secs 60° C. annealing/extension). Reference genes were selected based on expression evaluation in leaves of various developmental stages. Average Cq of the selected reference genes were used for ΔCq calculation. The 2^{(-ΔΔCq)} method was used to analyze the expression data.

Primers sets (each containing a forward primer, probe, and reverse primer) used for multiplex gene expression analysis are listed in Table 3. PtaAct2 (Potri.001G309500 sPta717), PtaEF1B-1 (Potri.001G224700 sPta717) were selected as reference genes.

TABLE 3
Probe Primers for Multiplex Gene Expression Analysis

| Gene Target | SEQ ID NO: | Sequence (5′ to 3′) |
|---|---|---|
| Plgg1 | 20 | CAGGTGTCAAGATTTGCTTCATC |
| | 21 | FAM/CTTTGCGTG/ZEN/GCGGGTTTCACAG/ |
| | | 3IABkFQ |
| | 22 | AGCGTCAGTCATTTCTGTCTT |
| PtACT2 | 23 | CTGCAATGTATGTTGCCATCC |
| | 24 | HEX/ACTGGCATA/ZEN/CAGGGAAAGGACAGC/ |
| | | 3IABkFQ |
| | 25 | CACACCATCACCAGAATCCA |
| PtEF1B-1 | 26 | CATCACTACCACTCCACACAA |
| | 27 | Quasar |
| | | 705/TCCTCTTTC/NOVa/AATCACACTGTTTATC |
| | | GCTCT/3BHQ |
| | 28 | TTTCAATTACTTGGAAGATGGGATG |
| CrGDH | 29 | AAGGCGTACGAGCTGATG |
| | 30 | Cy5/AACCCGGGC/TAO/GTCATCCTCAA/ |
| | | 3IAbRQSp |
| | 31 | GAGGGCTTCAGGAACTTGAT |
| ChMS | 32 | ATGGAATGCCGCAGAGAAG |
| | 33 | Texas Red- |
| | | XN/ATACTTGGTCGCCGGATCAAACCC/3TAbRQSP |
| | 34 | GATGCACATGTCCACTCAGAT |

### Example 5—Photosynthetic Efficiency Measurements

Transgenic seedlings were planted in pots and grown under greenhouse conditions, or planted in the field, with wild type (WT) and empty vector control (CT) seedlings used for comparisons. Weather data, including light intensity, air temperature, and precipitation, were measured.

Starting at one week post potting, morphology evaluations were carried out for greenhouse grown plants on a weekly basis including height measurement, leaf number counting, and phenotypic analysis. Eight weeks post potting, T0 plants went through a vegetative propagation cycle. Briefly, apical meristem was removed to release apical dominance and stimulate the formation of axillary buds leading to branch production for the next rounds of seedling production via stem cutting and hydroponic rooting. To indicate the genetic lineage of transgenic lines, cuttings from T0 plants were named C1 plants with C1. 1 referring to the apical cutting and C1.2 and so on referring to axillary cuttings. C1 cuttings normally established root systems after approximately two weeks and would be transferred to soil pots afterwards. Morphological evaluations were performed similarly with T0 plants.

Measurements of CO₂ assimilation were conducted using the LI-6800 portable fluorometer following the manufacturer's manual (licor.com/env/support/LI-6800/manuals.html, LI-COR, Lincoln, NE). Briefly, the LI-6800 CO₂ response program was used for measurement of the A-Ci curves with the reference CO₂ concentrations set to 400, 40, 20, 40, 60, 80, 100, 150, 200, 300, 400, 500, 700, 900, 1,100, 1,300, 1,500, and 2,000 ppm. Minimum and maximum wait times were set at 60 to 120 s based on the stability of the CO₂ net assimilation rate and the difference between sample and reference CO₂ concentrations. The reference and sample infrared gas analyzers (IRGAs) were matched before the measurement at each concentration. The measured leaf was allowed to acclimate at 400 ppm for ˜5 min. The light intensity was set at 1,500 μmol/m²/s.

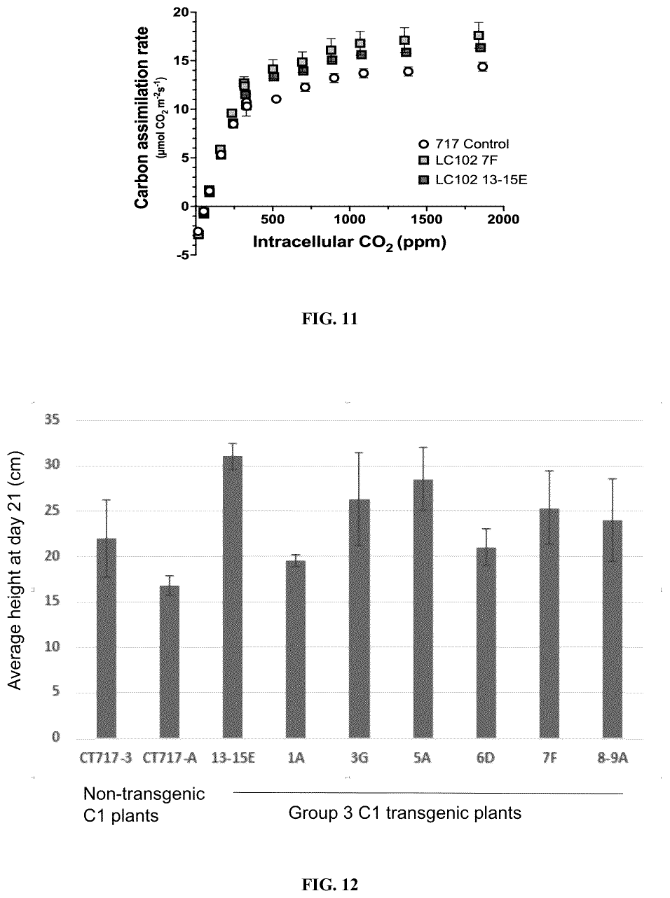

Photosynthesis rate is increased in the LC0102 plants. The CO₂ assimilation rate was measured on 18 weeks-old plants grown in a controlled environment using the LI-6800. As shown in FIG. 11, plants of two transgenic events, 13-15E and 7F, show elevated levels of carbon assimilation in saturated intracellular CO₂ concentrations. As discussed in Example 6, this elevated level of carbon assimilation rate led to faster growth of plants and increased biomass production.

### Example 6—Tree Growth and Biomass Measurements

Transgenic plants harboring the T-DNA from LC0102 grew faster. Measurement of plant growth was performed one week post soil transplantation on a weekly basis. Areas of growth measurement included plant height, root collar diameter (RCD), leaf number, SPAD readings, and other measurements. Plants of several LC0102 transgenic events started to display faster growth early, as reflected by height increases. FIG. 12 shows height measurement results of Group 3 transgenic plants 3 weeks after being transferred to soil pots. Transgenic events LC0102.13-15E, 5A, 7F, and 8-9A showed, respectively, a 40%, 14%, 27%, and 10% increase in height after 3 weeks growth in a controlled environment compared to non-transgenic control plant CT717-3.

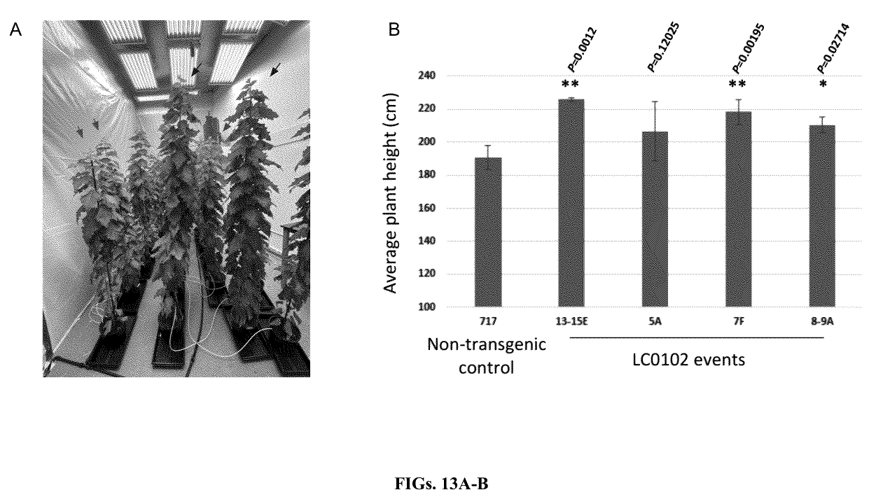

Transgenic plants harboring LC0102 grew into taller trees over time. Plants of four selected LC0102 events (13-15E, 5A, 7F, and 8-9A) along with non-transgenic control 717 were carried forward for extended growth evaluations in a high ceiling growth room. FIG. 13A is a photograph of plants grown in soil for 17 weeks. Transgenic plants were taller than non-transgenic control plants. FIG. 13B is a graph showing that the early observed growth advantage in LC0102 events continued to be reflected in plant height after 17 weeks. Compared to non-transgenic controls, events 13-15E, 7F, and 8-9A were significantly taller displaying a 18%, 15%, and 10% respective increase in height after 4 months, with p-values less than 0.01 (13-15E and 7F) or 0.05 (8-9A) (FIG. 12B). One ramet of event 5A was as tall as plants of 13-15E, however, the variation among the three ramets was too great to render a higher p-value.

The increased growth rate of these trees allows the reduction of the growth cycle from 10-12 to 6-7 years. Both carbon drawdown and wood production in forest trees are directly dependent on growth rate, so this also represents up to a 40% increase in both.

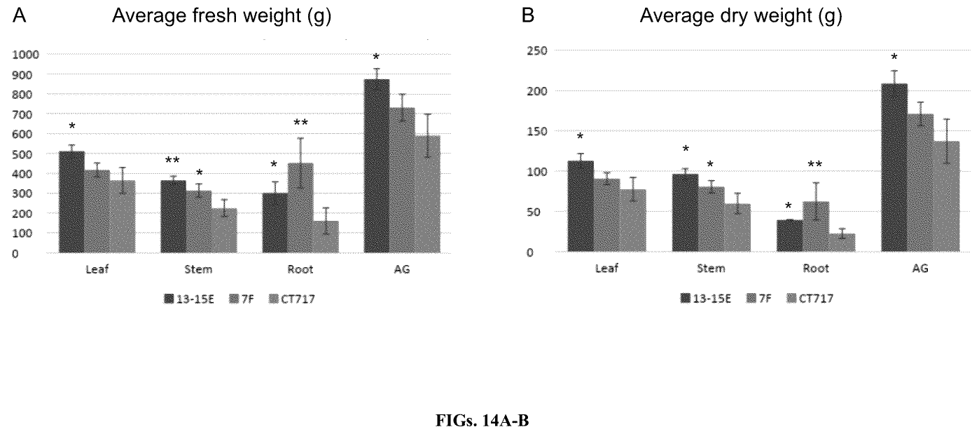

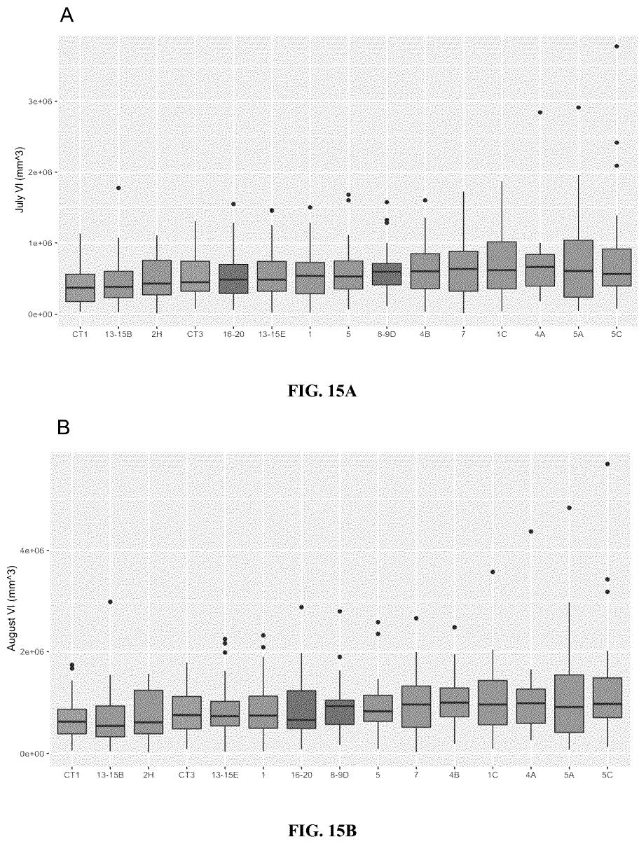

Transgenic plants harboring LC0102 had increased biomass production. Four selected LC0102 events, 13-15E, 5A, 7F, and 8-9A, along with non-transgenic CT717 plants were selected for morphology evaluation after an extended growth period in a controlled environment. At 21 weeks post-potting, plants were harvested for biomass measurements. Fresh weight (FW) and dry weight (DW) were measured for leaf, root, and stem tissues separately, and the data is summarized in FIGS. 14A-B. As shown, lead event 13-15E had significantly higher biomass production in all tissue types at both FW (FIG. 14A) and DW (FIG. 14B) levels, with above ground (AG) DW showing a 53% increase over that of non-transgenic CT717 control plants. Lead event 7F also had significantly higher biomass production in stem and roots at both FW and DW levels, but not in leaves. Event 5A and 8-9A did not show significantly higher biomass production.

The expression analysis of CI plants indicated Plgg1 expression levels of 47%, 46%, 57%, 73% compared to Plgg1 expression in control plants for events 13-15E, 5A, 7F, and 8-9A, respectively, as shown in FIG. 10. As shown in FIG. 14B, event LC0102.13-15E had a 53% increase in AG dry biomass production compared to the control. In Table 2, it was demonstrated that too much reduction in Plgg1 expression would lead to abnormalities in plant growth phenotypes. Taken together, in order to achieve significantly enhanced biomass production in engineered poplar trees with good morphology, a significant reduction in the expression level of Plgg1 (such as a 53% reduction (i.e., Plgg1 expression of 47% compared to Plgg1 expression in a control plant)) is beneficial. Slightly reduced Plgg1 expression did not lead to significantly enhanced biomass production. Reduction of Plgg1 expression by about 80% or more (i.e., Plgg1 expression of 20% or less compared to Plgg1 expression in a control plant) was found to be more likely to lead to growth abnormalities in engineered plants.

### Example 7—Plgg1 Expression in Field-Grown Trees

At least 30 ramets each of at least 10 different transgenic poplar tree events as well as the same or larger numbers of non-transgenic controls of the same genotype were acclimated outdoors, and planted in fields. Transgenic trees included ramets from events 1C, 2H, 4A, 5A, 7, 13-15B, 13-15E, and 5C. Control ramets were from were from non-transgenic control 717 (CT717-3), and transgenic escapes (i.e., not having a transgene) including 16-20 and 8-9D.

Trees were randomly selected from the field for gene expression studies after 12 months of growth. Samples were collected from leaf 10 (counting from the top) of each tree. Samples were processed with total RNA extraction, quantitation, normalization, and qRT-PCR analysis using primers shown in Table 3. Relative fold expression was determined using standard ΔΔCT method, and the results are shown in Table 4.

TABLE 4
Relative Expression Level of Plgg1 in Field-Grown Trees

| | | Average Fold Expression of Plgg1 | |
|---|---|---|---|
| | | Relative to Control (& % Plgg1 | Std |
| Event | # Trees | Expression Relative to Control) | deviation |
| 1C | 9 | 1.05 (105%) | 0.20 |
| 2H | 11 | 0.15 (15%) | 0.05 |
| 4A | 11 | 0.68 (68%) | 0.23 |
| 5A | 12 | 0.45 (45%) | 0.11 |
| 7 | 11 | 0.79 (79%) | 0.17 |
| 8-9D | 12 | 0.76 (76%) | 0.16 |
| 13-15B | 10 | 0.16 (16%) | 0.14 |
| 13-15E | 11 | 0.16 (16%) | 0.06 |
| 16-20 | 12 | 0.96 (96%) | 0.23 |
| CT717-3 | 11 | 0.97 (97%) | 0.14 |
| 5C | 15 | 0.49 (49%) | 0.11 |

Plgg1 expression level in non-transgenic CT717-3 trees was designated as 1.0 fold or 100%. With the exception of event 1C which has similar level of Plgg1 expression to the control, transgenic events in general had reduced Plgg1 expression ranging from 15% to 79% of that of the control. This represented an expression reduction of 21% to 85%. Events 13-15B, 13-15E, and 2H showed the greatest reduction of Plgg1 gene expression at around 84%.

A statistical analysis of Plgg1 gene expression in field-grown trees was conducted as shown in Table 5. Statistical difference was calculated by Dunnett's test for pairwise comparisons to CT717-3. The level of significance in difference between events are indicated with p-value and noted with asterisks: ns, p>0.05; *, p≤0.05; **, p≤0.01; ***, p≤0.001; ****, p≤0.0001.

TABLE 5
Statistical Analysis of Plgg1 Expression in Field-Grown Trees

| Dunnett's T3 | | | | | |
|---|---|---|---|---|---|
| Multiple | | | Below | | Adjusted |
| Comparisons Test | Mean Diff. | 95.00% CI of diff. | Threshold? | Summary | P Value |
| CT717-3 vs. 1C | −0.06089 | −0.3254 to 0.2036 | No | ns | 0.9946 |
| CT717-3 vs. 2H | 0.8575 | 0.7101 to 1.005 | Yes | **** | <0.0001 |
| CT717-3 vs. 4A | 0.3154 | 0.05697 to 0.5739 | Yes | * | 0.0119 |
| CT717-3 vs. 5A | 0.5532 | 0.3883 to 0.7182 | Yes | **** | <0.0001 |
| CT717-3 vs. 7 | 0.2076 | −0.0008545 to 0.4610 | No | ns | 0.0514 |
| CT717-3 vs. 8-9D | 0.2247 | 0.02400 to 0.4253 | Yes | * | 0.0221 |
| CT717-3 vs. 13-15B | 0.8455 | 0.6539 to 1.037 | Yes | **** | <0.0001 |
| CT717-3 vs. 13-15E | 0.8563 | 0.7109 to 1.002 | Yes | **** | <0.0001 |
| CT717-3 vs. 16-20 | 0.01099 | −0.2421 to 0.2641 | No | ns | >0.9999 |
| CT717-3 vs. 5C | 0.5108 | 0.3528 to 0.6689 | Yes | **** | <0.0001 |

### Example 8—Growth Performance of Engineered Trees in the Field

FIGS. 15A-B show graphs illustrating growth performance of engineered trees in the field. Stem volume index was generated from measurements of plant height and root collar diameter (V=diameter²×height). Data was collected from all trees in the field by the S. Strauss laboratory at Oregon State University, which included 35 to 44 trees per event.

FIG. 15A shows the stem volume index (mm³) measurements in July approximately 1 year after transplantation. FIG. 15B shows the stem volume index (mm³) measurements in August of the same year.

The transgenic events showed an upward trend in stem volume index compared to controls. Four transgenic events, 1, 13-15B, 13-15E, and 2H, showed stem volume index increases similar to transgenic escapes (16-20 and 8-9D) and non-transgenic controls (CT1 and CT3). Other transgenic events, including 4B, 7, 1C, 4A, 5A, and 5C had higher stem volume growth. Transgenic events 13-15B, 13-15E, and 2H had the lowest volume increases of all the transgenic events, and were also the events that had the most severely reduced Plgg1 expression under field growth conditions (expression levels were 16%, 16%, and 15%, respectively, of control Plgg1 expression, see Table 4). Whereas the top three performing transgenic events 5C, 5A, and 4A, had moderately reduced Plgg1 gene expression (expression levels were 49%, 45%, and 68%, respectively, of control Plgg1 expression, see Table 4).

### Example 9—Glycolate Dehydrogenase (CrGDH) and Malate Synthase (ChMS) Expression in Field Grown Transgenic Trees

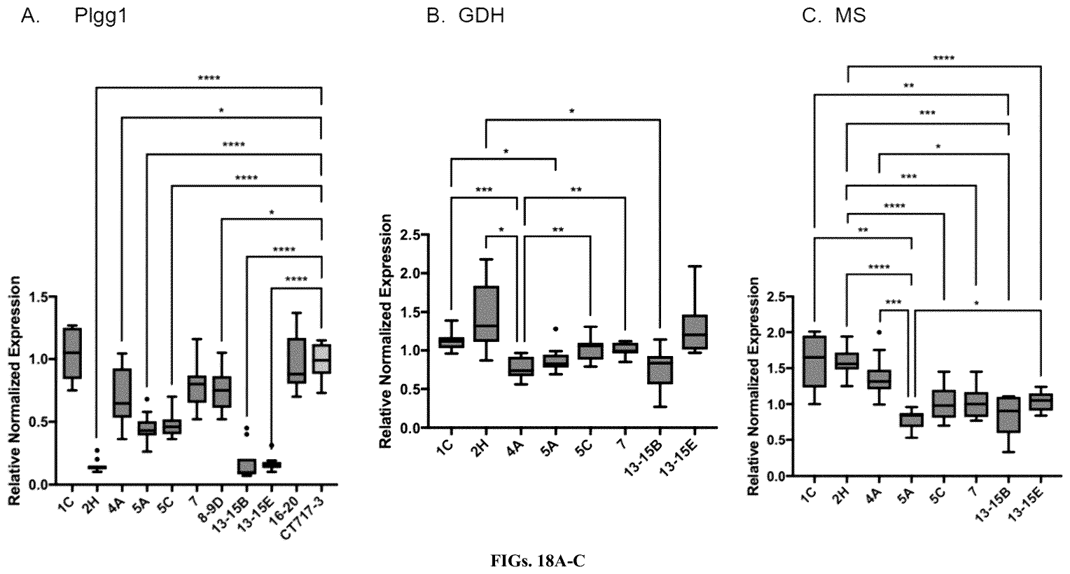

Relative expression levels of Glycolate Dehydrogenase (CrGDH) and Malate Synthase (ChMS) in various transgenic events and non-transgenic controls is shown in FIGS. 18B-C. In comparison, expression of endogenous Plgg1 in various transgenic events and non-transgenic controls is shown in FIG. 18A. CrGDH expression is similar among different events, with relative gene expression values ranging from 0.76 to 1.41 (FIG. 18B, and Table 6). Malate synthase expression ranges from a relative gene expression of 0.9 to 1.59 (FIG. 18C and Table 7). There is no expression of CrGDH nor ChMS in non-transgenic control CT717-3 and escapes as they do not contain these genes. Tables 6 and 7 show the number of ramets analyzed, the average normalized expression, and the standard deviation for CrGDH (Table 6) and ChMS (Table 7).

TABLE 6
GDH Expression in Various Events and Controls

| Event | Ramets | Average | Std deviation |
|---|---|---|---|
| 1C | 9 | 1.12 | 0.12 |
| 2H | 11 | 1.41 | 0.42 |
| 4A | 11 | 0.7553 | 0.13 |
| 5A | 12 | 0.88 | 0.15 |
| 7 | 11 | 1 | 0.08 |
| 8-9D | 12 | 0 | N/A |
| 13-15B | 10 | 0.76 | 0.26 |
| 13-15E | 11 | 1.31 | 0.37 |
| 16-20 | 12 | 0 | N/A |
| CT717-3 | 11 | 0 | N/A |
| 5C | 15 | 1.03 | 0.14 |

TABLE 7
MS Expression in Various Events and Controls

| Event | Ramets | Average | Std deviation |
|---|---|---|---|
| 1C | 9 | 1.57 | 0.37 |
| 2H | 11 | 1.59 | 0.20 |
| 4A | 11 | 1.3811 | 0.28 |
| 5A | 12 | 0.79 | 0.13 |
| 7 | 11 | 1.02 | 0.22 |
| 8-9D | 12 | 0 | 0.00 |
| 13-15B | 10 | 0.83 | 0.28 |
| 13-15E | 11 | 1.03 | 0.14 |
| 16-20 | 12 | 0 | 0.00 |
| CT717-3 | 11 | 0 | N/A |
| 5C | 15 | 1 | 0.22 |

### Example 10—Comparison Between Transgenic Event 13-15E and Controls

A comparison between transgenic event 13-15E and non-transgenic control over five experiments is shown in Table 8. Across five experiments conducted in growth room, greenhouse, and field conditions, transgenic event 13-15E was compared to non-transgenic controls. A strong correlation was identified between the degree of Plgg1 expression reduction and the growth performance of 13-15E.

Under experimental conditions when Plgg1 was severely reduced such as in the field and some greenhouse experiments (having only 8% or 16% of the Plgg1 expression level in controls), 13-15E plants did not show a significantly better growth advantage in stem volume increase compared to non-transgenic control plants (Table 8). In experiments when the stem volume difference of event 13-15E was significant, a less-severe degree of Plgg1 expression reduction in 13-15E plants was observed (having 47%, 30%, and 27% Plgg1 expression compared to controls) (Table 8). In all these experiments, expression levels of the other two transgenes CrGDH and ChMS were very similar (Table 8). These results further support the importance of a moderate down-regulation of endogenous Plgg1 expression. Reduction of Plgg1 expression to 20% or less of the expression level of Plgg1 in a control plant, can lead to severe detrimental morphological effects and/or lack of growth benefits to the plants when co-expressed with malate synthase and glycolate dehydrogenase.

TABLE 8
Comparison of Gene Expression in Event 13-15E in Different Environments

| | | | | Plgg1 % | |
|---|---|---|---|---|---|
| | | Expression | Expression | Expression | Stem Volume |
| | | of | of | Relative | Significantly |
| | | CrGDH | ChMS | to | Higher Than |
| Experiment | Location | (dCq) | (dCq) | Controls | Controls? |
| C2 Exp | Growth | −5.50 ± 0.35 | −3.82 ± 0.65 | 27% | Yes |
| | Room | | | | |
| | (location 1) | | | | |
| Field | Field | −6.65 ± 0.37 | −3.71 ± 0.19 | 16% | No |
| Year 2 | (location 3) | | | | |
| Pilot 1 | Green- | −5.12 ± 0.18 | −4.05 ± 0.18 | 30% | Yes |
| | house | | | | |
| | (location 2) | | | | |
| Pilot 2 | Green- | −5.80 ± 0.48 | −4.14 ± 0.40 | 8% | No |
| | house | | | | |
| | (location 2) | | | | |
| C1 Exp | Growth | NA | NA | 47% | Yes |
| | Room | | | | |
| | (location 1) | | | | |

### Example 11—Transgenic Events Have Higher Photosynthetic Activity & Stomatal Conductance Under Drought Conditions

Transgenic plants, including 4 plants of event 13-15E, and 1 plant of event 7, as well as 4 plants of non-transgenic control CT7-717-3 were grown for twelve weeks in a controlled environment. Plants were watered to saturation before the start of the experiment. Across 4 days, plants were watered overnight with minimal water to keep them alive, but not saturated. Each plant was measured with a Licor LI-6800 photosynthesis system twice a day with full A-Ci (CO₂ assimilation) curves. Soil moisture was recorded with a soil moisture probe during each measurement. Soil moisture was categorized into drought severity levels: None (>9.5), Mild (7.5-9.49), Medium (5.5-7.49), Severe (<5.49).

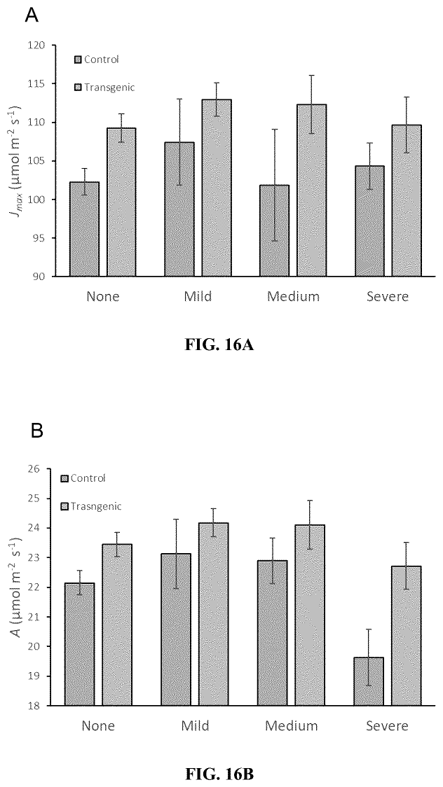

Jₘₐₓ (maximum rate of photosynthetic electron transport) and Aₘᵤₓ (maximum carbon assimilation rate) are indicators for photosynthetic activity in plants. Higher values indicate higher activity. As shown in FIGS. 16A-B, under drought conditions, the transgenic trees were able to maintain higher level of photosynthetic activity. Even under the most severe drought conditions, Aₘₐₓ in the transgenic plants was only slightly reduced, whereas it was drastically reduced in the non-transgenic plants.

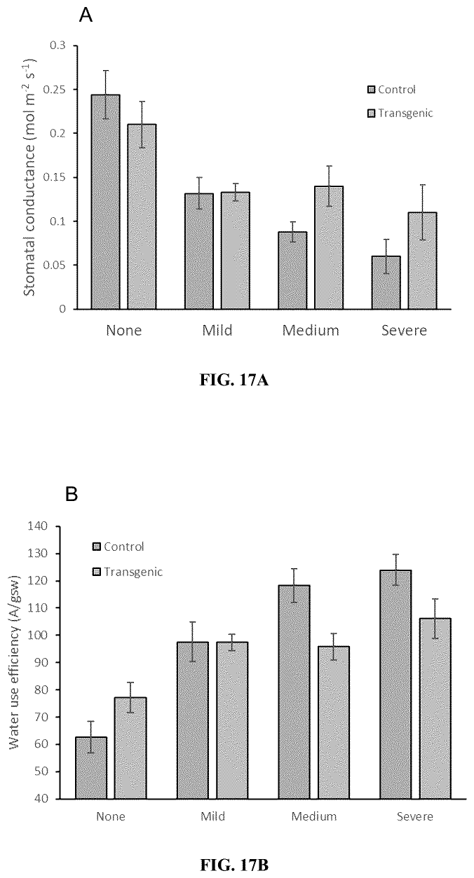

As shown in FIGS. 17A-B, the transgenic events showed higher stomatal conductance compared to non-transgenic controls under drought conditions. This was correlated with higher photosynthetic activity. On the contrary, non-transgenic plants showed a drastic reduction in stomatal conductance resulting from natural response to drought conditions when stoma in leaves are closing to reduce water loss due to transpiration, leading to artificially higher water use efficiency. Otherwise, the transgenic plants had higher water use efficiency as suggested by higher photosynthetic activity.

### Example 12—Plgg1 in Other Species for RNAi & Other Exemplary Sequences

RNAi constructs for endogenous Plgg1 suppression in other tree species can be made in a similar fashion as described in the Examples above. SiFi or other software can be used to optimize sequence selection for use in the RNAi constructs. Examples of Plgg1 sequences from tree species are given in Table 9.

A full length coding sequence for Pinus taeda (Loblolly pine) (SEQ ID NO:35) (PITA_51316, which is hereby incorporated by reference in its entirety) is shown in Table 9. Exemplary Plgg1 RNAi polynucleotide fragments for use in Pinus taeda are shown below. A sense Pinus taeda Plgg1 fragment for use in RNAi (SEQ ID NO:36) is:

ATGGCTTCAT CCCACCAATT CCAAACTTCA TGGCTGCTCA CACGGAGACC GTTGGGAGAC
GCCTCTTTGC GTTTATATCA ATTACCCCGC AGGACCACAA AAGAGCAGAG GAAAAATTAT
GACAATTGCA GCTGTTGGGC GCAGAGGAGA AGAGAGGCAG CTGTCGCTGC AGCAATCAGA
ATTAATGGTG AGCGTAAGTT CATGTTCACG GGGTTTGGCA GGCAAAGGCC AAGGCGAAGT
CATGAAATAA TAATG

An antisense sense Pinus taeda Plgg1 fragment for use in RNAi (SEQ ID NO:37) is:

CATTATTATT TCATGACTTC GCCTTGGCCT TTGCCTGCCA AACCCCGTGA ACATGAACTT
ACGCTCACCA TTAATTCTGA TTGCTGCAGC GACAGCTGCC TCTCTTCTCC TCTGCGCCCA
ACAGCTGCAA TTGTCATAAT TTTTCCTCTG CTCTTTTGTG GTCGTGCGGG GTAATTGATA
TAAACGCAAA GAGGCGTCTC CCAACGGTCT CCGTGTGAGC AGCCATGAAG TTTGGAATTG
GTGGGATGAA GCCAT

Other exemplary Plgg1 sequences for use in methods disclosed herein include Plgg1 from Populus alba (SEQ ID NO:38) (GenBank Accession No. XM_035032358.1, which is hereby incorporated by reference in its entirety), Populus tremula (SEQ ID NO:39) (Potra000595g04516, which is hereby incorporated by reference in its entirety), Populus euphratica (SEQ ID NO:40) (GenBank Accession No. XM_011029786.1, which is hereby incorporated by reference in its entirety), and Picea abies (SEQ ID NO:41) (MA_92186g0010, which is hereby incorporated by reference in its entirety). SEQ ID NOs:38-41 are shown in Table 9.

Additional exemplary malate synthase and glycolate dehydrogenase sequences are shown in Table 9. These include Populus trichocarpa malate synthase (SEQ ID NO:42) (Potri.015G092000, which is hereby incorporated by reference in its entirety), Populus tremula malate synthase (SEQ ID NO:43) (Potra001425g12048, which is hereby incorporated by reference in its entirety), Eucalyptus grandis malate synthase coding sequence (SEQ ID NO:44) and protein sequence (SEQ ID NO:45) (GenBank Accession No. XM_010039145, which is hereby incorporated by reference in its entirety), Chlamydomonas reinhardtii glycolate dehydrogenase coding sequence (SEQ ID NO:46) and protein sequence (SEQ ID NO:47) (GenBank Accession No. DQ647436, which is hereby incorporated by reference in its entirety), and Volvox carteri f. nagariensis glycolate dehydrogenase coding sequence (SEQ ID NO:48) and protein sequence (SEQ ID NO:49) (GenBank Accession No. XM_002946413, which is hereby incorporated by reference in its entirety).

TABLE 9
Plgg1 Sequences & Other Exemplary Sequences

| | SEQ ID | |
|---|---|---|
| Source | NO: | SEQUENCE (5′ to 3′) |
| Pinus taeda | 35 | ATGGCTTCAT CCCACCAATT CCAAACTTCA TGGCTGCTCA |
| Plgg1 | | CACGGAGACC GTTGGGAGAC GCCTCTTTGC GTTTATATCA |
| (PITA_51316) | | ATTACCCCGC AGGACCACAA AAGAGCAGAG GAAAAATTAT |
| | | GACAATTGCA GCTGTTGGGC GCAGAGGAGA AGAGAGGCAG |
| | | CTGTCGCTGC AGCAATCAGA ATTAATGGTG AGCGTAAGTT |
| | | CATGTTCACG GGGTTTGGCA GGCAAAGGCC AAGGCGAAGT |
| | | CATGAAATAA TAATGGTGAA AGCCTTTGAA AAGGATGCCA |
| | | GAGGCCTCCC GGATGGCTCC CTCAAGGTGG GTCGTGTGGC |
| | | GCATTTGGTG GTTTCGTTGG GCATAATTCT GGCCATCGAC |
| | | AAGGCCTTGA AAAGGCTCTT CACGGCTGCC ATGATAAAGT |
| | | TCCCTAGCGC ATTGTTTGGA ATGTTCTGCA TATTTTCCGT |
| | | GCTGTCAATC CTCGACGTCA TCAGACCGGC AGCGGCAACG |
| | | GCTCTACTGA ACTTTTTTGA GCCGGCAACA ACGTTCATAC |
| | | AGAGATGGCT TCCGCTCTTC TACGTGCCGT CGCTGGTAGT |
| | | GTTGCCCTTA GCTGTGAAGG GAATCCCTGC TTCTTCCGGA |
| | | ATGAAGATTG GCGCAATTCT TGTTGGAGGC TGGGCAGCAT |
| | | CACTCTATGT TGCAGGGTAC ACAGCCATTG CCGTAAGAAA |
| | | GATGGTCAGA ACAAAAATGA TAGCAGCAGA GCCCATGCCA |
| | | AAGCCTTCTC CGTTTTCAAC AATAGAGATT TGGTTCTGGA |
| | | CTATTATCTT GTCAGTTTCA TTTATATTGA CTGTTCTTTC |
| | | ACCTCTTGCA TTTGGAACTT ATGCTCGAAC ATGCCTTCCG |
| | | TGTCTTTTAG CGGCAACTGC GTTAGGATTC ATCCTTGGAT |
| | | CTGGATTTCC ACCTACTGCC AAGAAGCTAC TACATCCAAT |
| | | TATTACTTGT GCCCTATCTG CGGATTTGGC AGCCTTTGCT |
| | | TGTGGGATCT CCACAGGATT GGGCTTTGAA TCCACGCTAG |
| | | GGGCCTATTT AACTAAAAGT GCAACCAATC CTGGTGCTGG |
| | | TGATATCTTG ATGGGATTTC TTGGATCTGT GATTCTTTCA |
| | | TTCGCTTTCT CAATGTTCCG TCAGAGAAAG CTTGTCAAAC |
| | | GGCATGCTGC GGAGATCTTC TCAGCTGTGG TGGTTTCAAC |
| | | AATTTTTTCT CTGTACTCAA CAGCAGCTAT GGGCCGAATG |
| | | ATTGGACTGG AACCAACCTT AACTGTTTCC ATTTTGCCAA |
| | | GATGTATTAC TGTTGCACTA GCATTGAGCA TTGTCTCGCT |
| | | TTTTGATGGG CAATATAACT CTCGGGCGGA TTGTCCTTCA |
| | | GGGTCTAGGG ATGAAAGAAA ATGA |
| Populus alba | 38 | ATGGC TACTCCTTTA GTCGCTCTTT CCGTTCAACT |
| Plgg1 | | CTGTCATCAC CACTCAAAAC AATATTCTTT CAAATCACAA |
| (XM_035032358.1) | | TCACAATCAC GTGTCAATAG GGATTTCCGT ACAAAAACAC |
| | | TACTTGGTGT TTACAATGGA TTCCAAAATG TATATAACCA |
| | | ATCTTATTTT CATAAGCCTT GGGCACCCAT TAGAGTTCTT |
| | | GAGCCTAATT CAAGGTTCTT GCAAATGGGT CCTCAAGAAA |
| | | CCTGCTCTAG TCGAGGAATT TCCAAGAAAT CTATGAGCTC |
| | | AGAAGGCAGT ACCAGTACTA GCTCTTCATC TATTTCTCAA |
| | | CAGGTGATTG GGATCTTGCA TTTGCTTGTT TCACTTGGGA |
| | | TTATCCTTGC AATGGATAAG TTGTTGAAAA AGGCATTTGT |
| | | GGCTGCTGCT ATCAAGTTTC CAAGTGCTCT GTTTGGCATG |
| | | TTCTGCATAT TCTCAGTTTT AGTGATTCTT GATATAACTA |
| | | TTCCGGCTGC TGCAACAAGC TTAATGAACT TCTTTGAGCC |
| | | AGCACTGTTA TTCATTCAGA GATGGCTTCC GTTGTTCTAC |
| | | GTTCCATCAT TGGTTGTTTT GCCTCTCTCT GTTAAAGATA |
| | | TCCCTGCTGC ATCAGGTGTC AAGATTTGCT TCATCACAGC |
| | | TGGAGGGTGG CTGGCGTCAC TTTGTGTGGC GGGTTTCACA |
| | | GCTATTGCTG TGAGAAAAAT GGTGAAGACA GAAATGACTG |
| | | ACGCTGAGCC TATGGCGAAA CCCTCTCCTT TTTCTCCATT |
| | | GGAAATGTGG GCTTGGAGTG GGGTCTTCCT TGTATCATTT |
| | | GTTGTCGCAT TATTATATAG AACGGCATTG GGGACTGCAG |
| | | CCAGAACATG CCTTCCTTTC CTACTAGCGT CCACCGTGCT |
| | | AGGCTATATG GTTGGTTCTG GGTTACCATC TGGTGTTAAG |
| | | AAGGTTTTCC ATCCCATTAT TTGTTGTGCA CTATCTGCAG |
| | | ATTTGGCAGC GTTGGCCTTT GGGTACCTTT CCCAATCTGG |
| | | ACTTGATCCC GTTCTAGGAT ATTACCTTAC AAAAGTTTCA |
| | | TCTAATCCTG GAGCTGGTGA TGTGTTAATG GGATTCTTGG |
| | | GACCTGTCAT TCTTTCTTTT GCCTTCTCAA TGTTCAAGCA |
| | | GCGAAAGCTT GTTAAGAGAC ATGCAGCTGA GATTTTCACG |
| | | TCGGTCATTG TTTCAACACT ATTTTCATTG TATTCAACGG |
| | | CTCTCGTGGG ACGTCTAGTT GGGTTAGAAC CAACGTTGAC |
| | | TGTATCCATT ATTCCCAGAT GTATAACCGT GGCATTAGCC |
| | | CTCAGCATTG TGTCATTCTT TGAAGGTGCC AATTCATCTC |
| | | TCACAGCTGC TGTAGTTGTT GTAACTGGTC TCATTGGAGC |
| | | AAATTTTGTA CAAGCAGTGC TTGATAAATT AAGTTTTCGT |
| | | GATCCCATTG CTCGAGGAAT AGCTACTGCT TCCAGTGCTC |
| | | ATGGACTGGG AACTGCAGCA TTGTCAGCCA AGGAACCTGA |
| | | GGCACTTCCA TTTTGTGCCA TTGCTTATGC TCTCACTGGT |
| | | ATATTTGGTT CATTGTTTTG TTCAGTTCCT GCAGTTAGGC |
| | | AAAGCTTACT TGCAATAATT GGCTGA |
| Populus | 39 | ATGGCTACTC CTTTAGTCGC TCTTTCCGTT CAACTCTGTC |
| tremula Plgg1 | | ATCACCACTC AAAACAATAT TCTTTCAAAT CACAATCACG |
| (Potra000595g04516) | | TGTCAATAGG GATTTCCGTA CGAAAACACT ACTTGGTGTT |
| | | TACAATGGAT TCCAAAATGT ATATAACCAA TCTTATTTTC |
| | | ATAAGCCTTG GGCACCCATT AGAGTTCTTG AGCCTAATTC |
| | | AAGGTTCTTG CAAATGGGTC CTCAAGAAAC CTGCTCTAGT |
| | | CGAGGAATTT CCAAGAAATC TATGAGCTCA GAAGGCAGTA |
| | | CCAGTACTAG CTCTTCATCT ATTTCTCAAC AGGTGATTGG |
| | | GATCTTGCAT TTGCTTGTTT CACTTGGGAT TATCCTTGCA |
| | | ATGGATAAGT TGTTGAAAAA GGCATTTGTG GCTGCTGCTA |
| | | TCAAGTTTCC AAGTGCTCTG TTTGGCATGT TCTGCATATT |
| | | CTCGGTTTTA GTGATTCTTG ATATAACTAT TCCGGCTGCT |
| | | GCAACAAGCT TAATGAACTT CTTTGAGCCA GCACTGTTAT |
| | | TCATTCAGAG ATGGCTTCCG TTGTTCTACG TTCCATCATT |
| | | GGTTGTTTTG CCTCTCTCTG TTAAAGATAT CCCTGCTGCA |
| | | TCAGGTGTCA AGATCTGCTT CATCACAGCT GGAGGGTGGC |
| | | TGGCGTCACT TTGCGTGGCG GGTTTCACAG CTATTGCTGT |
| | | GAGAAAATTG GTGAAGACAG AAATGACTGA CGCTGAGCCT |
| | | ATGGCGAAAC CCTCTCCTTT TTCTCCATTG GAAATGTGGG |
| | | CTTGGAGTGG GGTCTTCCTT GTATCATTTG TTGTCGCATT |
| | | ATTATATAGA ACGGCATTGG GGACTGCAGC CAGAACATGC |
| | | CTTCCTTTCC TACTAGCGTC CACCGTGCTA GGCTATATGG |
| | | TTGGTTCTGG GTTACCATCT GGTGTTAAGA AGGTTTTCCA |
| | | TCCCATTATT TGCTGTGCAC TATCTGCAGA TTTGGCAGCG |
| | | TTGGCCTTTG GGTACCTTTC CCAATCTGGA CTTGATCCCG |
| | | TTCTAGGATA TTACCTTACA AAAGTTTCAT CTAATCCTGG |
| | | AGCTGGTGAT GTGTTAATGG GATTCTTGGG ACCTGTCATT |
| | | CTTTCTTTTG CCTTCTCAAT GTTCAAGCAG CGAAAGCTTG |
| | | TTAAGAGACA TGCAGCTGAG ATTTTCACGT CGGTCATTGT |
| | | TTCAACACTA TTTTCATTGT ATTCAACGGC TCTCGTGGGA |
| | | CGTCTAGTTG GGTTAGAACC AACGTTGACT GTATCCATTA |
| | | TTCCCAGATG TATAACCGTG GCATTAGCCC TCAGCATTGT |
| | | GTCATTCTTT GAAGGTGCCA ATTCATCTCT CACAGCTGCT |
| | | GTAGTTGTTG TAACTGGTCT CATTGGAGCA AATTTTGTAC |
| | | AAGCAGTGCT CGATAAATTA AATTTTCGTG ATCCCATTGC |
| | | TCGAGGAATA GCAACTGCTT CCAGTGCTCA TGGACTGGGA |
| | | ACTGCAGCAT TGTCAGCCAA GGAACCTGAG GCACTTCCAT |
| | | TTTGTGCCAT TGCTTATGCT CTCACTGGTA TATTTGGTTC |
| | | ATTGTTTTGT TCAGTTCCTG CAGTTAGGCA AAGCTTACTT |
| | | GCAATAATTG GCTGA |
| Populus | 40 | ATGGCTACTC CTTTAGTCGC TCATTCCGTT CTACTCTGTC |
| euphratica | | ATCACCACTC AAAACAATAT CCTTTCAAAT CGCAATCACG |
| Plgg1 | | TGTCGATAGG GATTTCCGTA CGAAAACACT ACTTGGTGTT |
| (XM_011029786.1) | | TACAATGGAT TCCAAAATGT ATATAACCAA TCTTATTTTC |
| | | ATAAGCCTTG GGCACCCATT AGAGTTCTTG AGCCTAATTT |
| | | AAGGTTCTTG CAAATGGGTC CTCAAGAAAC CTGCTCTAGT |
| | | CGACGAATTT CCAAGAAATC TATGAGCTCA GAAGGCAGTA |
| | | CCAGTAGTAC TAGCTCTTCA TCTATTTCTC AACAGGTGAT |
| | | TGGGATCTTG CATTTGCTTG TTTCACTTGG GATTATCCTT |
| | | GCAATGGATA AGTTGTTGAA AAAGGCATTT GTGGCTGCTG |
| | | CTATTAAGTT TCCAAGTGCT CTATTTGGCA TGTTCTGCAT |
| | | ATTCTCAGTT TTAGTCATTC TTGATATAAC TATTCCGGCT |
| | | GCTGCAACAA GCTTAATGAA CTTCTTTGAG CCAGCACTGT |
| | | TATTCATTCA GAGATGGCTT CCATTGTTCT ACGTTCCATC |
| | | ATTGGTTGTT TTGCCTCTCT CTATTAAAGA TATCCCTGCT |
| | | GCATCAGGTG TCAAGATTTG CTTCATCACA GCTGGAGGGT |
| | | GGTTGGCGTC ACTTTGTGTG GCGGGTTTTA CAGCTATTGC |
| | | TGTGAGAAAA ATGGTGAAGA CAGAAATGAC TGATGCTGAG |
| | | CCTATGGCGA AACCCTCTCC TTTTTCTCCA TTGGAAATGT |
| | | GGGCTTGGAG TGGGGTCTTC CTTGTATCAT TTGTTGTCGC |
| | | CTTATTATAT AGAACGGCAT TGGGGACTGC AGCCAGAACA |
| | | TGCCTTCCTT TCCTACTAGC ATCCACCGTG CTAGGCTATA |
| | | TGGTTGGTTC TGGGTTACCA TCTGGTGTTA AGAAGGTTTT |
| | | CCATCCCATT ATTTGTTGTG CACTATCTGC AGATTTGGCA |
| | | GCGTTGGCCT TTGGGTTCCT TTCCCAATCC GGACTTGATC |
| | | CCGTTCTAGG ATATTACCTT ACAAAAGTTT CATCCAATCC |
| | | TGGAGCTGGT GATGTGTTAA TGGGATTCTT GGGATCTGTC |
| | | ATTCTTTCTT TTGCTTTCTC AATGTTCAAG CAGAGAAAGC |
| | | TTGTTAAGAG ACATGCAGCT GAGATTTTCA CGTCAGTCAT |
| | | TGTTTCAACA CTATTTTCAT TGTATTCAAC GGCTCTCGTG |
| | | GGACGCCTAG TTGAGTTAGA ACCAACATTG ACTGTATCCA |
| | | TTATTCCCAG ATGTATAACC GTGGCATTAG CCCTCAGCAT |
| | | TGTGTCATTC TTTGAAGGTG CCAATTCATC TCTCACAGCT |
| | | GCTGTAGTTG TTGTAACTGG TCTCATTGGA GCAAATTTTG |
| | | TACAAGCAGT GCTCGATAAA TTAAATTTTC TTGATCCCAT |
| | | TGCTCGAGGA ATAGCAACTG CTTCCAGTGC TCATGGACTG |
| | | GGAACTGCAG CATTGTCAGC CAAGGAACCT GAGGCACTTC |
| | | CATTTTGTGC CATTGCTTAT GCTCTCACTG GTATATTTGG |
| | | TTCATTGTTT TGTTCAGTTC CTGCAGTTAG GCAAAGCTTA |
| | | CTTGCAATAA TTGGCTGA |
| Picea abies | 41 | ATGGCCCTGG AAGTTAAACC TGCTAAAAAT AGTAAATTGC |
| Plgg1 | | CAAAATTGCC AGAAAACTAT TTTGAGCCAG TCGGAAAAGG |
| (MA_92186g0010) | | TGGGTGGCCA CTGATATCCT CTTGGATCAA ACGGCATGCT |
| | | GCAGAGATCT TCTCAGCTGT GGTGGCTTCA ACAATTTTTT |
| | | CTCTGTACTC AACAACAGTT GTAGGTCGAA TGATTGGAAT |
| | | GGAACCAACC TTAACTGTTT GCATTTTGCC AAGATGTATT |
| | | ACTGTTGCAT TAGCACTGAG CATTGTCTCA CTTTTTGATG |
| | | CTGCAAATCC TTCCCTTACA GTAGCTGTCA TGGTTTTAAC |
| | | GGGATTGGTT GGGGAAAATT TTGCACAAAT ATTTCTCGAC |
| | | AAGCTTGGTT TGGATGATTC TATTGCACGA GGAATGGCTA |
| | | TAGCCTTCAG TGCTCATGGA TTGGGAACGA CAGCATTGTC |
| | | AGCAAAAGAA CCAGAAGCAT TGCCTTTCTG TGCAATTTCC |
| | | TATGCATTAA CTGCGGAGGC CATGGCTGGA GATGGTGGAT |
| | | CGGAGCGCCT TGACGAAGCG GGAGTCGCTG GGTTAGGCGA |
| | | GCAACAAGAT CTGCAAGAAT TGTACATATT TCCACATCGA |
| | | TTACCTATTG CTGCTCTGAC GGAGCCTCTC GTCCTACTCA |
| | | ACAGGACCTA CAGCAACAGG GAGGTTCTCG GGATCATGAC |
| | | GGTGCTAACC ATTGTTATCG TGTTTCTCAC GAGCATTGGG |
| | | TCGTTACTCA TCTCGGTGCT TATGGTCGGA TTGGGTATCG |
| | | TTTGTGTCCA TGGTGCATTT AGGGTTTCCG AAGATCTGTT |
| | | TTTGGACGAG CAGGAGCAGG CGGTCAGATT CTTGTCCTTC |
| | | CTCGGTGGCG GTGGATCCCA TCCCCCTGTT GCTATCCATG |
| | | TTTGA |
| Populus | 42 | GAAACACTCT CATACACGTT AAAAACCATA CAACAAAGAA |
| trichocarpa | | GAGTGGCATT GCTTAAAAAA CAGTCAGATC AATAATGGGA |
| Malate | | CCTGCAACTT ATGAGAATCA CTATCCACCG CCCAATATGA |
| Synthase | | AGAAGTTTGG CTATGATGCT CCTGAAGGAG TTGAAATTAG |
| (Potri.015G092000) | | AGGCAGATAT GACGGAGAGT TTGCAGTGAT CCTTACCAAG |
| | | GATGCTTTGC AATTTGTTGC TGATTTGCAG AGAGAGTTTA |
| | | GGAACCGCAT CAAGTATGCA ATGGAGTGTC GCAAGGAAGC |
| | | CAAAAGGAGG TACAGTGAGG GGGCTTTACC AGGGTTTGAT |
| | | CCAGCTACAA GGTACATAAG GGAGGGGGAG TGGACATGTG |
| | | CACCTGTCCC TCCAGCTGTT GCTGATAGGA AGGTGGAGAT |
| | | TACAGGTCCA GTGGAGAGGA AAATGATCAT CAATGCACTC |
| | | AATTCTGGAG CTAAAGTTTT CATGGCTGAC TTTGAAGATG |
| | | CACTGTCACC AAGTTGGGAG AACCTTATGA GAGGACAAGT |
| | | TAACCTGAAG GACGCTGTCG CTGGGACTAT TACCTTTCAC |
| | | GACAAGGTCA GGAACAGGGT TTATAAGCTG AATAACCAGA |
| | | CAGCCAAGTT GTTTGTCCGC CCACGAGGTT GGCATCTACC |
| | | TGAGGCGCAC ATTCTCATTG ATGGAGAACC AGCAACTGGT |
| | | TGCCTTGTGG ACTTTGGCCT TTACTTTTAC CACAACTATG |
| | | CAGCATTCCG TCGGATTCAG GGTGCAGGGT TCGGGCCTTT |
| | | CTTCTATCTC CCTAAGATGG AGAATTCAAG GGAGGCTAAG |
| | | ATATGGAACT GTGTGTTCGA GAAAGCAGAG AAGATGGCAG |
| | | GAATTGAAAG GGGAAGCATC AGGGCCACTG TCCTAATCGA |
| | | AACACTTCCA GCTGTTTTCC AAATGAATGA AATCCTCTAC |
| | | GAACTAAGGG ATCACTCTGT TGGCTTGAAC TGCGGAAGAT |
| | | GGGATTACAT CTTCAGCTAT GTCAAGACAT TCCAGGCTCA |
| | | CCCAGATCGC CTGCTACCAG ACAGGGTTCA AGTTGGCATG |
| | | ACTCAGCACT TCATGAAGAG TTACTCTGAT CTCCTCATCT |
| | | GGACATGCCA TAGGCGTGGT GTCCATGCGA TGGGCGGCAT |
| | | GGCAGCTCAG ATTCCAATCA GAGACGATCC AGAGGCGAAT |
| | | AATGAAGGAC TAGAACTCGT GCGAAAGGAC AAGCTAAGAG |
| | | AGGTTCGAGC AGGACATGAT GGAACATGGG CAGCCCACCC |
| | | TGGACTTATC CCAGCTTGCA TGGAAGTCTT CTCAAACAAC |
| | | ATGGGAAACA CGCCAAACCA AATCCAGTCC ATGAAGCGCG |
| | | AAGATGCATC AACCATTACA GAGGAAGACC TCTTACAGAG |
| | | GCCAAGAGGG TCCCGTTCGT TGGAAGGTCT CCGCCTAAAC |
| | | ACCCGTGTAG GAATCCAGTA CTTAGCAGCA TGGCTAACTG |
| | | GAACTGGCTC AGTCCCTCTT TACAACCTCA TGGAAGACGC |
| | | TGCCACTGCA GAAATCAGCA GAGTTCAGAA CTGGCAATGG |
| | | CTTAAATATG GAGTGGAACT TGACGGAGAT GGCCTTGGAG |
| | | TGAAAGTTAA CAATGATCTG TTCGGGAAAG TGGTGGAAGA |
| | | GGAAATGGCT AGGATTGAAA GGGAAGTTGG GAAAGAGAAA |
| | | TTCAAGAGGG GAATGTACAA GGAGGCCTGC AAGATTTTCG |
| | | CAAGGCAATG CACAGCACCT ACACTGGATG ATTTTCTTAC |
| | | CCTGAATGCT TATGATAATA TCGTCATCCA TCACCCTATG |
| | | GGATCATCAT CCCGCCTCTA AGTCATGAGT TTCATCGCTT |
| | | GTTTTTAATT TCCTGGGATT GATGTGTGCC CTCGTGTTCT |
| | | ACTCTACTAC TACAACTTGA ATAAAATCCT TTCTATCTGT |
| | | TTGAACTTAA GTTCCACCCA TTA |
| Populus | 43 | GTTTAATCAA ATCTTACGCA CATAAAGCCA TAACGATAAT |
| tremula | | GCTCCTTGTA CCTATCCACT TGCCTCCACT CTCCCTTTAT |
| Malate | | ATATTTCTAC TCTTGAGCTT ACCCTTGAAA CACTCTCATA |
| Synthase | | CACGTTAAAA ACCATACAGT AAAGAAGAAT CGCGTTGCTA |
| (Potra001425g12048) | | AAAAAACAGT GAGATCAATA ATGAGACCTG CAACTAATGG |
| | | GAATCCGGAT GCACCGCCCG ATATGAAGAA GTTTGGCTAT |
| | | GATTCTCCTG AAGGAGTTGA AGTTAGAGGC AGATATGACG |
| | | GCGAGTTTGC AGTGATCCTT ACCAAGGATG CTTTGCAATT |
| | | TGTTGCTGAT TTGCAGAGAG AGTTTAGGAA CCGCATCAAG |
| | | TATGCAATGG AGTGTCGCAA GGAAGCCAAA AGGAGGTACA |
| | | GTGAGGGGGC TCTACCAGGG TTTGATCCAG CTACAAGGTG |
| | | CATAAGGGAG GAGGAGTGGA CATGTGCACC TGCCCCTCCA |
| | | GCTGTTGATG ATAGGAAGGT GGAGATTACA GGTCCAGTGG |
| | | AGAGGAAAAT GATCATCAAT GCCCTCAATT CTGGAGCGAA |
| | | AGTTTTCATG GCTGACTTTG AAGATGCGCT CTCACCAAGC |
| | | TGGGAGAACC TTATGAGAGG ACAAGTTAAC CTGAAGGATG |
| | | CTGTTGATGG GACTATTACC TTTCACGACA AGGTCAGGAA |
| | | CAGGGTTTAT AAGCTGAATA ATCAGACAGC CAAGTTGTTT |
| | | GTCCGCCCAC GAGGTTGGCA TCTACCTGAG GCGCACATTC |
| | | TCATTGATGG AGAACCTGCA ACTGGTTGCC TTGTGGACTT |
| | | TGGCCTTTAC TTTTACCACA ACCATGCAGC ATTCCGTCGG |
| | | AATCAGGGTG CAGGGTTCGG GCCTTTCTTC TATCTCCCTA |
| | | AGATGGAGAA TTCAAGGGAG GCTAAGATAT GGAACTGTGT |
| | | GTTTGAGAAA GCAGAGAAGG TGGCAGGAAT TGAAAGGGGA |
| | | AGCATCAGGG CCACTGTCCT TATCGAAACA CTTCCAGCTG |
| | | TCTTCCAAAT GAATGAAATC CTCCACGAAC TAAGGGATCA |
| | | CTCTGTTGGG TTGAACTGCG GAAGATGGGA TTACATCTTC |
| | | AGCTATGTCA AGACATTCCA GGCTCACCCA GATCGCCTGC |
| | | TACCAGACAG AGTTCAAGTT GGCATGACTC AGCACTTCAC |
| | | GAAGAGCTAC TCCGATCTCC TCATCTGGAC ATGCCATAGG |
| | | CGTGGTGTGC ACGCGATGGG CGGCATGGCA GCTCAGATTC |
| | | CTATCAGAGA CGATCCAGAG GCGAATAAGG AAGCACTGGA |
| | | GCTCGTGCGA AAGGACAAGC TAAGAGAGGT TCGAGCAGGA |
| | | CATGACGGAA CATGGGCAGC CCACCCTGGA CTTATCCCAG |
| | | CTTGCATGGA AGTCTTCTCA GACAACATGG GAAACACGCC |
| | | AAACCAGATC CAAACCATGA AGCGCGAAGA TGCATCAACC |
| | | ATAACAGAGG AAGACCTCTT ACAGAGACCA AGAGGGTCCC |
| | | GTTCATTGGA AGGTCTCCGC CTAAACACCC GTGTGGGAAT |
| | | CCAGTACCTA GCAGCATGGC TAACTGGAAC TGGATCGGTC |
| | | CCTCTTTACA ACCTCATGGA AGATGCCGCC ACCGCAGAAA |
| | | TCAGCAGAGT TCAGAACTGG CAATGGCTTA AATATGGAGT |
| | | GGAACTTGAC GGAGATGGCC TTGGAGTGAG AGTTAACAAT |
| | | GATCTGTTCG GGAAAGTGGT GGAGGAGGAG ATGGCTAGGA |
| | | TTGAAAGGGA GGTCGGGAAA GAGAAGTTCA AGAGGGGAAG |
| | | GTACAAGGAG GCCTGCAAGA TTTTCGCAAG GCAATGCACA |
| | | GCACCTACAC TAGATGATTT TCTTACACTG AATGCTTATG |
| | | AAAATATCGT CAGCCATCAC CCTATGGGAT CATCACCCCG |
| | | CCTCTAAGTC ACGAGTTTCA TCGCTTGTTT TTAATTTCCA |
| | | GGGATTGATG TGTGCCCTCG TGTTCCACTC TACTACTACA |
| | | AGTTATCTAC AACTTGAATA AAATCTTTTC TACCTGTTTG |
| | | AACTTAAGTT CCACCCTTTA CTTGCACTTA TTTTATTT |
| Eucalyptus | 44 | ATGATGGAT CTCGGGTCGT TCACGTACCA AGCACCAGCA |
| grandis | | ACCAACAAGG CCAACCCCGC GTCCGGCTAC GACGTCCCCG |
| Malate | | AGGGAGTGGA CATCAGAGGG CGTTACGATG CCCAGTTCGC |
| Synthase | | CAGGATCCTC NACCGGGACG CCTTGCAGTT CGTCGCCGAT |
| (XM_010039145) | | CTGCAGAGGG AGTTCAGGGG CCACATCAAG TACGCCATGG |
| | | AGTGTCGCCG CAACGCCAAG CAGCGCTACA ACGCCGGCGC |
| | | GCTGCCTGGG TTCGACCCCG CTACGAGGTA CGTGAGGGAA |
| | | GGCGAGTGGA CGTGCTCGCT GGTCCCACCA GCCGTGGCCG |
| | | ACCGGAGGGT GGAGATCACT GGCCCTGTCG AGCGCAAGAT |
| | | GATCATCAAC GCGCTCAACT CTGGCGCCAA AGTTTTCATG |
| | | GCAGATTTCG AAGATGCGCT ATCCCCGAGC TGGGAGAACC |
| | | TGATGAGAGG GCAAGTGAAC CTGAAGGATG CGGTGGATGG |
| | | GACCATAACC TTCCACGACA AGGCCCGAAA CAGGGTCTAC |
| | | AAGCTCAATG ACCAGATTGC CAAGCTCTTT GTCCGGCCCC |
| | | GAGGTTGGCA CTTGCCTGAG TCTCACATCC TCATTGACGG |
| | | TGAGCCTGCA ACCGGTTGCC TCTTCGACTT TGGCCTCTAC |
| | | TTCTTCCACA ACTACGCTTC ATTCAGGAAA ATGCAGGGTG |
| | | AGGGGTTCGG GCCCTTCTTC TATCTCCCTA AGATGGAGCA |
| | | TTCCAGGGAA GCGAAGATAT GGAACAGCGT CTTTGAGAGG |
| | | GCAGAGAAAA TGGCGGGCAT CGAGAGAGGG AGCATAAGGG |
| | | CAACGGTATT GATAGAGACA CTACCTGCAG TGTTCCAGAT |
| | | GGATGAGATA CTGTACGAGC TGAGGGATCA CTCGGTCGGA |
| | | TTGAACTGCG GAAGATGGGA CTACATCTTC AGCTATGTCA |
| | | AGACCTTCCA GGCTCATCCC GATCGCCTAC TGCCTGACAG |
| | | GTTTCAGGTC GGGATGACCC AGCACTTCAT GAGGTCTTAC |
| | | TCTGATCTCC TCATCCGGAC TTGCCATCGG CGCGGTGTTC |
| | | ATGCCATGGG AGGCATGGCG GCTCAAATCC CGATCAGAGA |
| | | TGATCCGGTG GCCAATGAGG CGGCGCTGGA ACTAGTAAGG |
| | | AAAGACAAGC TCAGAGAAGT CAGGGCCGGG CATGACGGGA |
| | | CATGGGCTGC TCACCCGGGG TTAATCCCGG CTTGTATGGA |
| | | AGTCTTCACG AATCACATGG GCAATGCGCC ACACCAGATC |
| | | CAATCAGTCA AGAGAGAGGA CGCAGCCAAC ATAACCGAGG |
| | | AAGACCTCCT CCAGAGGCCG AGAGGGGCCC GAACTTTGGA |
| | | AGGCCTCCGG CTCAACACTC GGGTTGGGAT CCAATACCTG |
| | | GCAGCCTGGC TCACAGGGAC AGGGTCGGTC CCACTCTACA |
| | | ATCTCATGGA GGATGCGGCA ACTGCCGAGA TCAGCCGAGT |
| | | CCAGAACTGG CAGTGGCTGA AGTATGGTGT GGAGCTGGAC |
| | | GGTGATGGGG TTGGAGTGAG AGTGAGCTTG GATCTATTCG |
| | | CGAGGGTGGT GGAGGAAGAG GTGGCCAGGA TTGAGNNAGA |
| | | GGTTGGGAAG GACAAGTTCA AGAAGGGGAT GTACAAGGAG |
| | | GCTGGCAAGA TGTTCACGAG ACAATGCACG GCGCCAGTGT |
| | | TGGACGATTT CCTGACGCTC GATGCCTACA ATCACATCGT |
| | | CGTGCATCAT CCTAGGGGGT CTTCCAAGCT CTGA |
| Eucalyptus | 45 | MMDLGSFTYQ APATNKANPA SGYDVPEGVD IRGRYDAQFA |
| grandis | | RILRDALQFV ADLQREFRGH IKYAMECRRN AKQRYNAGAL |
| Malate | | PGFDPATRYV REGEWTCSLV PPAVADRRVE ITGPVERKMI |
| Synthase | | INALNSGAKV FMADFEDALS PSWENLMRGQ VNLKDAVDGT |
| (XM_010039145) | | ITFHDKARNR VYKLNDQIAK LFVRPRGWHL PESHILIDGE |
| | | PATGCLFDFG LYFFHNYASF RKMQGEGFGP FFYLPKMEHS |
| | | REAKIWNSVF ERAEKMAGIE RGSIRATVLI ETLPAVFQMD |
| | | EILYELRDHS VGLNCGRWDY IFSYVKTFQA HPDRLLPDRF |
| | | QVGMTQHFMR SYSDLLIRTC HRRGVHAMGG MAAQIPIRDD |
| | | PVANEAALEL VRKDKLREVR AGHDGTWAAH PGLIPACMEV |
| | | FTNHMGNAPH QIQSVKREDA ANITEEDLLQ RPRGARTLEG |
| | | LRLNTRVGIQ YLAAWLTGTG SVPLYNLMED AATAEISRVQ |
| | | NWQWLKYGVE LDGDGVGVRV SLDLFARVVE EEVARIEEVG |
| | | KDKFKKGMYK EAGKMFTRQC TAPVLDDFLT LDAYNHIVVH |
| | | HPRGSSKL |
| Chlamy- | 46 | ATGCCACGCG GCCAGGGCAA GCGCCTGGCT CAGCTCCTTG |
| domonas | | GAGCTCAGCT GAAGCAGTAC GCAGCGGAGG TGCGTGGCAT |
| reinhardtii | | CAGCACAGCT GGTGGCGCTT CTCGCGGTGG AGCTCGAGGA |
| Glycolate | | CCTGCATCCC CTAGCTCGCT AGAGCAGCAG ACGCGCCAGG |
| dehydrogenase | | TCGCTCAGGT TGCTGTTCAG CAGTCGACTC AGCAGGCAGT |
| (DQ647436) | | GAAGGTCGTT GTGCCGGCCA TCAAAGTAGA CCTGGTTGGT |
| | | GCGGTCAGCT CGGTGTCTGA GAGCGACAAG GTGGAGCCGG |
| | | GTGTGTTCAA GAACGTGGAT GGCCACCGCT TCGAGGACGG |
| | | TCGCTATGCC GCTTTTGTTG AGGAGATTAC AAAGTTTATC |
| | | CCCAAGGAGC GCCAGTACTC GGACCCCGTG CGCACATTCG |
| | | CGTATGGCAC GGATGCCTCC TTCTACCGGC TTAACCCGAA |
| | | GCTGGTAGTG AAGGTGCACA AGGAGGACGA GGTCCGCCGC |
| | | ATCATGCCCA TCGCGGAGCG GCTGCAGGTC CCTATCACCT |
| | | TCCGCGCGGC CGGCACGTCG CTGTCTGGGC AGGCAATTAC |
| | | CGACTCGGTG CTCATTAAGC TGAGCCACAC GGGCAAGAAC |
| | | TTCCGCAACT TTACCGTGCA CGGCGACGGT AGCGTGATCA |
| | | CGGTGGAGCC GGGCCTCATT GGCGGCGAGG TGAACCGCAT |
| | | CCTGGCGGCA CACCAGAAGA AGAACAAGCT GCCCATCCAG |
| | | TACAAGATCG GACCCGACCC CTCCTCCATC GACAGCTGCA |
| | | TGATCGGCGG CATCGTGTCC AACAACAGCA GCGGCATGTG |
| | | TTGCGGCGTG AGCCAGAACA CCTACCACAC GCTGAAGGAC |
| | | ATGCGGGTGG TGTTCGTAGA CGGAACGGTG CTGGACACGG |
| | | CCGACCCCAA CTCGTGCACC GCCTTCATGA AGAGCCACCG |
| | | CTCGCTGGTG GATGGCGTCG TGAGCCTGGC GCGCCGCGTG |
| | | CAGGCCGACA AGGAGCTGAC GGCGCTCATC CGCCGCAAGT |
| | | TCGCCATCAA GTGCACCACC GGCTACTCCC TGAACGCGCT |
| | | GGTGGACTTC CCGGTGGACA ACCCCATTGA GATCATCAAG |
| | | CACCTCATCA TCGGCAGCGA GGGCACGCTG GGCTTCGTCA |
| | | GCCGCGCCAC CTACAACACC GTGCCCGAGT GGCCCAACAA |
| | | GGCCTCGGCC TTCATCGTGT TCCCGGACGT GCGCGCCGCC |
| | | TGCACCGGCG CCTCGGTGCT GCGCAACGAG ACGTCCGTGG |
| | | ACGCGGTGGA GCTGTTTGAC CGCGCCAGCC TGCGCGAGTG |
| | | CGAGAACAAC GAGGACATGA TGCGCCTGGT GCCCGACATC |
| | | AAGGGCTGCG ACCCCATGGC GGCAGCGCTG CTGATCGAGT |
| | | GCCGCGGCCA GGACGAGGCC GCACTGCAGA GCCGCATTGA |
| | | GGAGGTGGTG CGCGTGCTGA CGGCGGCGGG CCTGCCCTTC |
| | | GGCGCCAAGG CCGCGCAGCC CATGGCCATC GACGCCTACC |
| | | CCTTCCACCA CGACCAGAAG AACGCCAAGG TCTTCTGGGA |
| | | CGTGCGCAGG GGCCTGATCC CCATTGTGGG CGCGGCGCGC |
| | | GAGCCCGGCA CATCCATGCT GATCGAGGAC GTGGCCTGCC |
| | | CCGTGGACAA GCTGGCCGAC ATGATGATCG ACCTGATCGA |
| | | CATGTTCCAG CGCCACGGCT ACCACGACGC CTCCTGCATC |
| | | GGCCACGCGC TCGAGGGCAA CCTTCACTTG GTGTTCTCGC |
| | | AGGGCTTCCG CAACAAGGAG GAGGTGCAGC GCTTCAGCGA |
| | | CATGATGGAG GAGATGTGCC ACCTGGTGGC CACCAAGCAC |
| | | TCGGGCAGCC TCAAGGGCGA GCACGGCACG GGCCGCAACG |
| | | TGGCGCCGTT CGTGGAGATG GAGTGGGGCA ACAAGGCGTA |
| | | CGAGCTGATG TGGGAGCTCA AGGCGCTGTT CGACCCCAGC |
| | | CACACCCTCA ACCCGGGCGT CATCCTCAAC CGCGATCAGG |
| | | ACGCGCACAT CAAGTTCCTG AAGCCCTCGC CCGCGGCCTC |
| | | GCCCATCGTC AACCGCTGCA TCGAGTGCGG CTTTTGCGAG |
| | | TCCAACTGCC CCTCGCGCGA CATCACGCTC ACGCCGCGCC |
| | | AGCGCATCTC CGTGTACCGC GAGATGTACC GCCTCAAGCA |
| | | GCTGGGCCCG GGCGCCAGCG AGGAGGAGAA GAAGCAGCTG |
| | | GCGGCCATGA GCAGCTCGTA CGCCTACGAC GGCGAGCAGA |
| | | CGTGCGCGGC GGACGGCATG TGCCAGGAGA AGTGCCCCGT |
| | | CAAGATCAAC ACGGGAGACC TGATCAAGTC GATGCGTGCC |
| | | GAGCACATGA AGGAGGAGAA GACCGCCAGC GGCATGGCAG |
| | | ACTGGCTGGC CGCCAACTTC GGCGTCATCA ACTCCAACGT |
| | | GCCGCGCTTC CTCAACATCG TCAACGCCAT GTACAGCGTG |
| | | GTGGGCTCGG CGCCTCTGTC CGCGATCAGG CGCGCGCTCA |
| | | ACGCCGCCAC CAACCACTTC GTGCCGGTGT GGAACCCCTA |
| | | CATGCCCAAG GGCGCGGCGC CGCTCAAGGT GCCCGCCCCG |
| | | CCGGCGCCGG CAGCTGCTGA GGCCTCGGGC ATCCCGCGCA |
| | | AGGTGGTGTA CATGTCCAGC TGCGTGACGC GCATGATGGG |
| | | CCCCGCCGCC TCCGACACCG AGACGGCGGC GGTGCACGAG |
| | | AAGGTGATGA GCCTGTTCGG CAAGGCCGGC TACGAGGTGA |
| | | TCATCCCCGA GGGCGTGGCC AGCCAGTGCT GCGGCATGAT |
| | | GTTCAACAGC CGCGGCTTCA AGGACGCCGC CGCCAGCAAG |
| | | GGCGCGGAGC TGGAGGCGGC GCTGCTCAAG GCCTCGGACA |
| | | ATGGCAAGAT GCCCATCGTC ATCGACACCT CGCCCTGCCT |
| | | GGCGCAGGTG AAGAGCCAGA TCAGCGAGCC GTCGCTGCGC |
| | | TTCGCGCTGT ACGAGCCGGT TGAGTTCATC CGGCACTTCC |
| | | TGGTGGACAA GCTGGAGTGG AAGAAGGTGC GCGACCAGGT |
| | | GGCCATCCAC GTGCCCTGCT CCTCCAAGAA GATGGGCATC |
| | | GAGGAGTCCT TCGCGAAGCT GGCGGGCCTG TGCGCCAACG |
| | | AGGTGGTGCC CTCGGGCATT CCTTGCTGCG GCATGGCGGG |
| | | CGACCGCGGC ATGCGCTTCC CCGAGCTGAC CGGCGCCTCG |
| | | CTGCAGCACC TCAACCTGCC CAAGACCTGC AAGGACGGCT |
| | | ACTCCACCAG CCGCACCTGC GAGATGTCGC TCAGCAACCA |
| | | CGCCGGCATC AACTTCAGGG GCCTGGTGTA CCTGGTGGAT |
| | | GAGGCCACGG CGCCTAAGAA GCAGGCCGCC GCTGCCAAGA |
| | | CCGCGTAA |
| Chlamy- | 47 | MPRGQGKRLA QLLGAQLKQY AAEVRGISTA GGASRGGARG |
| domonas | | PASPSSLEQQ TRQVAQVAVQ QSTQQAVKVV VPAIKVDLVG |
| reinhardtii | | AVSSVSESDK VEPGVFKNVD GHRFEDGRYA AFVEEITKFI |
| Glycolate | | PKERQYSDPV RTFAYGTDAS FYRLNPKLVV KVHNEDEVRR |
| dehydrogenase | | IMPIAERLQV PITFRAAGTS LSGQAITDSV LIKLSHTGKN |
| (DQ647436) | | FRNFTVHGDG SVITVEPGLI GGEVNRILAA HQKKNKLPIQ |
| | | YKIGPDPSSI DSCMIGGIVS NNSSGMCCGV SQNTYHTLKD |
| | | MRVVFVDGTV LDTADPNSCT AFMKSHRSLV DGVVSLARRV |
| | | QADKELTALI RRKFAIKCTT GYSLNALVDF PVDNPIEIIK |
| | | HLIIGSEGTL GFVSRATYNT VPEWPNKASA FIVFPDVRAA |
| | | CTGASVLRNE TSVDAVELFD RASLRECENN EDMMRLVPDI |
| | | KGCDPMAAAL LIECRGQDEA ALQSRIEEVV RVLTAAGLPF |
| | | GAKAAQPMAI DAYPFHHDQK NAKVFWDVRR GLIPIVGAAR |
| | | EPGTSMLIED VACPVDKLAD MMIDLIDMFQ RHGYHDASCI |
| | | GHALEGNLHL VFSQGFRNKE EVQRFSDMME EMCHLVATKH |
| | | SGSLKGEHGT GRNVAPFVEM EWGNKAYELM WELKALFDPS |
| | | HTLNPGVILN RDQDAHIKFL KPSPAASPIV NRCIECGFCE |
| | | SNCPSRDITL TPRQRISVYR EMYRLKQLGP GASEEEKKQL |
| | | AAMSSSYAYD GEQTCAADGM CQEKCPVKIN TGDLIKSMRA |
| | | EHMKEEKTAS GMADWLAANF GVINSNVPRF LNIVNAMYSV |
| | | VGSAPLSAIS RALNAATNHF VPVWNPYMPK GAAPLKVPAP |
| | | PAPAAAEASG IPRKVVYMSS CVTRMMGPAA SDTETAAVHE |
| | | KVMSLFGKAG YEVIIPEGVA SQCCGMMFNS RGFKDAAASK |
| | | GAELEAALLK ASDNGKIPIV IDTSPCLAQV KSQISEPSLR |
| | | FALYEPVEFI RHFLVDKLEW KKVRDQVAIH VPCSSKKMGI |
| | | EESFAKLAGL CANEVVPSGI PCCGMAGDRG MRFPELTGAS |
| | | LQHLNLPKTC KDGYSTSRTC EMSLSNHAGI NFRGLVYLVD |
| | | EATAPKKQAA AAKTA |
| Volvox | 48 | ATGACAGCTA TGCAGGCCAA CGGCGGCAGC GGCATTAGCG |
| carteri | | ATGTTACTGC CACCGCCGCG ACCACGGGGG AGACCTCAGG |
| f. nagariensis | | TGGGGAAGCG GGGCCAGTGG GACGGCTGGA GGCGGCCACG |
| Glycolate | | GCGCCAACGG ACGCGGCGGC GCTGGCCTAC TCAGATGCGG |
| dehydrogenase | | GTGCCACCGG CGTAGACGGC CTAGATGAGA CGCGCAAGTT |
| (XM_002946413) | | TGTGTTCGCG GAGGAATGGG GCTTTTCGCG CGTGGGAGGT |
| | | GACCTGCCGG ATGGCACCAC GCCCGCCACG TTTGCCGACA |
| | | TGTTTCCCGA GCGTTTCTTC GCCTTCAACC CCCTGCGCGC |
| | | GGTAACGGCG GTGGCGCTAC CACTGGTCGC TATGGCCGTT |
| | | GGCTACGGCT GGCTGTGGTA CATGCACTCA ATCTGCCCCG |
| | | CCTGGCAGCA GCTCGCGTGC GCAGCCCTTA TCGGCACCGC |
| | | TTACACGGGC CTCTTTAAAG TCGCCCACGA GTGTGCGCGC |
| | | TTCAGCTTTC TGCCTGAAGC TCCTCGCCTC CAAGATGCGC |
| | | TAGGGCTTGT CCTCATGCTG CCGTCGTTGT ACCCCTTCAC |
| | | CTCCTGGCGC CTCCATTACA TCCACCATAT GGCGCACCTC |
| | | AACATGCTGT GGGAGGATAC CTTTGGCTGG CACCCGTACA |
| | | CGAAGCTGCA GCTGGCCACG GAGCTCCTCT GTGGGCAGCA |
| | | CTGGCTCCTG GCACTGCGGC GCCTGGCGCT GACCACACCG |
| | | CTAAAGCTGT TTGCGTCCGT GGGCCACTGG CTTCGCTCCT |
| | | TTGACGGGCT GGACTTGAAG CGCTTCCACA AAGACACCTA |
| | | CTGGGCTGTG CTGGCGGGCT GGTCTGGGCC CATCCTGTTT |
| | | TTGGGTTTCG GCATCCCGGC TATCATTAAG ATGTTTGGCT |
| | | TGTCAGTCCG TCTAGCCGAC CGGGCTTGGG GGGGCTTAGT |
| | | AAGCGGCTAC CTGCTTCCCT GGCTGGTCTT CCACTTCTGG |
| | | CTGTCCACTC TATCGCTCCT GCAGCACACC GCACCGCATA |
| | | TTCCATTCCG CGCCGAGGAC GCGGGCTACG ACTCCGGCCG |
| | | CGCGGCCATC TGCGGCACCG TGACGGTGCG GCTGCCACGG |
| | | CCTCTGGAGC TGCTGCTTAA CGACGCCAAC TACTCCCTAC |
| | | CGCAGCTGGT GGCCCCTGGG ATGCCAGTGT TTCACGCGCG |
| | | GGAGGCGTAT GAGTACATGC GGGAGCGGTT GCTGCCGTAC |
| | | CTGACGGAGG CGACGCTTAG CGTAAAACTA CTGACCAACC |
| | | ATATTACCAA GTGGCAGGTT TATGATGCGG AAAAGGAGAC |
| | | GTACCTTCCT CTGGATGAGG TCATGCAGGA GCTCGAACGC |
| | | GACATTGCCA CTCTTATGGA GGAGGCTGAG CAGGCGGAGG |
| | | AGGAGGAGAA AGTTTCAGGG GGGCCAAGTG GCGGAGCGGT |
| | | AGAGGGGCAG GGCAAGCGCC TGGCGCAGCT GTTGGGTGCG |
| | | CAGCTTAAGC AATACATTTC GGAGGTTCGC AGCATCAGCA |
| | | GCTCAAATGC TGCTGGTCGG GGAGCGTTGG GCAGCGGCCA |
| | | CACGCCTACG ACGCAGGTCG TCACTGGCCA GCAAACACGC |
| | | TCTCATGCCC AGGTCGCAGT CCAGCAGGCT ACACAGCAGC |
| | | ACTTGAAAGT TGTGGTGCCG GCCATCAGGA CCGATCTGGT |
| | | CGGAGCGGTG TCTATGGTGT CTGATGGCGA GAAAGTGGAG |
| | | CCAGGCGTCT TCAAGAACAT TGACGGACAC CGCTTCGACG |
| | | ACGGTCGCTA CCAGGCTTTT ATTGAGGAAA TCACAAAATT |
| | | TATCCCCAAG GAGCGGCAGT ACACCGACCC TGTCCGCACG |
| | | TTCGCGTATG GCATCGACGC GAGTTTCTAC CGCCTTAACC |
| | | CAAAGCTGGT CGTTAAGGTC CACAATGAAG AAGAAGTTCG |
| | | GAAGATTTTG CCCATCGCGG AGCGGCTCAA GGTGCCGGTC |
| | | ACCTTCCGTG CTGCGGGTAC ATCCCTCTCG GGCCAGGCTA |
| | | TCACCGACTC GGTGCTGATT AAGCTCAGCC ACACCGGCAA |
| | | GAATTTCCGG AACTACACCG TGCACGGCGA CGGCAGCAGC |
| | | ATCACAGTGG AGCCGGGCCT CATCGGTGGC GAGGTTAACC |
| | | GCATCCTGGC GGCGTATCAG AAGAAGCACA AGCTGCCCAT |
| | | TCAGTACAAG ATAGGCCCAG ACCCGTCTTC CATCGACTCT |
| | | TGCATGATCG GCGGCATCGT GGCCAACAAC AGCAGCGGCA |
| | | TGTGCTGCGG TGTCAGCCAA AACACATATC ACACGCTCAA |
| | | GGATATGCGG GTGTTGTTTG TGGACGGCAC GGTACTGGAT |
| | | ACGGCAGATC CGGACAGCTG CGCGGCCTTC CTCAAGAGCC |
| | | ATAAGGCCCT GGTGGACGGC GTCGTGGACC TGGCGCGGCG |
| | | TGTGCAGGCT GACAGGGAGC TGACGGCTCT TATCCGTCGC |
| | | AAGTTTGCCA TCAAATGCAC CACCGGCTAT TCGCTGAATG |
| | | CGCTGGTGGA TTTCCCGCTG GACAACCCCA TTGAGATTAT |
| | | CAAGCACGTC ATCATTGGCA GCGAGGGCAC TCTGGGCTTC |
| | | GTGTCGCGGG CCACCTACAA CACTGTGCCC GAGTGGCCCA |
| | | ACAAGGCCTC GGCCTTCATC GTCTTCCCGG ACGTGCGGGC |
| | | GGCATGCATG GGTGCCTCCG TGCTCCGAAA TGAGACCTCT |
| | | GTGGACGCAG TAGAGCTGTT TGACAGGGCT TCCCTACGCG |
| | | AGTGCGAGAA CAATGAGGAC ATGATGCGGC TGGTGCCAGA |
| | | CATCAAGGGC TGCGACCCCA TGGCTGCCGC GGTGCTGATT |
| | | GAGTGCCGTG GCCAGGACGA GGCGGCGCTG CAGGGTCGTA |
| | | TTGAGGAGGT GGTCCGTGCT CTTACCTCGG CGGGCCTGCC |
| | | GGTGGGAGCG AAGGCGGCTG AACCCCGCCC CATCACCGCC |
| | | TATCCCTTCC ACCACGATGC CAAGAACTCG AAAGTCTTCT |
| | | GGGACGTGCG CAGGGGTCTG ATTCCGATCG TGGGTGCCGC |
| | | CCGTGAGCCG GGTACCTCCA TGCTTATTGA GGACGTCGCT |
| | | TGCCCCGTGG ACAAGCTGGC GGACATGATG ATTGACCTGA |
| | | TTGACATGTT CCAGCGCTAT GGCTACAACG ACGCTTCGTG |
| | | CTTCGGCCAC GCGCTGGAGG GCAACCTGCA CCTGGTCTTC |
| | | TCTCAGGGCT TCCGTACCAA GGAGGAGGTA CAGCGATTTG |
| | | CGGACATGAT GGAGGAGATG TGCTACATCG TGGCCACTAA |
| | | ACACAGCGGC AGCCTGAAGG GCGAGCATGG CACAGGCCGC |
| | | AACGTGGCTC CCTTCGTTGA GATGGAATGG GGCAGCAAGG |
| | | CGTATGAGCT GATGTGGGAG CTCAAGGAGC TGTTTGACCC |
| | | CAAGTACACG CTCAACCCCG GCGTCATTCT CAACCGGGAC |
| | | CCCGATGCGC ACATCAAATT CCTCAAGCCT TCGCCTGCTG |
| | | CTTCGCCAAT CGTTAATCGC TGCATCGAGT GCGGCTTCTG |
| | | CGAGTCCAAC TGCCCCTCCC GGGACATCAC GCTCACGCCC |
| | | CGCCAGCGCA TCGCCGTGTA CCGTGAGATG TACCGCCTGA |
| | | AGCAGCTGGG CCCGGCCGCC AGCGAGGAAG AGAAGCAGCA |
| | | GTTGGCAGCC ATTACAAGCT CCTATGCTTA CGATGGCGAG |
| | | CAGACGTGCG CCGCCGACGG CATGTGCCAG GAGAAGTGCC |
| | | CCGTCAAGAT AAACACAGGC GACATGATCA AGGCTCTCCG |
| | | CGCCGAGCAC ATGAAGGAGG CCAAGTCCGC CAGTGGTGCT |
| | | GCCATGTGGA TGGCTAACAA CTTCGGAGTC CTCAACGCCA |
| | | CAGTGCCGCG CTTCCTGAAC CTCGTTAATT TGGCGCACAG |
| | | TATCCTGGGC CCCAAGCCGC TTGAGGCCAT CTCCCGGGCG |
| | | CTGAACGCTG CCACCAATCA CCTGGTGCCC GTCTGGAACC |
| | | CCTACATGCC CAAGGGCGCC GCGCCGCTGA AGGTGCCCGC |
| | | TCCGTTCCCT GCACCCGCGG CGTCCTCTGG CGGCATCCCC |
| | | CGCCGAGTGG TGTACATGCC CTCTTGCGTG ACCCGCATGA |
| | | TGGGCCCCGC GGCGAGCGAC AAGGAGACGG CCTCTGTGCA |
| | | TGAGAAGATT TTGAGCCTCT TCAACAAGGC CGGCTACGAG |
| | | GTCATTGTTC CAGAGGGTGT CAGCAGCCAA TGTTGCGGCA |
| | | TGATGTTCAA CAGCCGTGGC TTCAAGGACG CCGCTGCCGC |
| | | CAAGGGTGCC GATCTGGAAG CGGCGCTGCT CAAGGCATCC |
| | | GATAACGGCA AGATCCCCAT CGTTATGGAC ACTTCGCCAT |
| | | GCCTGGCGCA GGTTAAGAGC CAAATCTCGG AGCCAGCGCT |
| | | ACGCTTCGCG CTGTACGAAC CGGTTGAGTT CATCAGGCAC |
| | | TTCCTGGTTG ACAAACTTGA GTGGCGCAAG GTGCGGGAGC |
| | | AGGTGGCCAT CCATGTGCCC TGCTCATCCA AGAAGATGGG |
| | | CATCGAGGAG TCCTTCGCCA AGCTGGCAGG ATTGTGTGCG |
| | | CACGAGGTTG TCCCGTCGGG CATTCCCTGC TGCGGTATGG |
| | | CTGGCGACCG TGGAATGCGC TACCCGGAGC TGACTGGCTC |
| | | CTCACTGCAG CACCTAAACC TGCCCAAGAG CTGCAGCGAT |
| | | GGCTACTCCA CCAGTCGCAC CTGCGAGATG TCGTTGTCCA |
| | | ACCACTCGGG CATCAACTTC AGGGGCCTGG TCTACTTGGT |
| | | GGACGAGGCG ACTAGCCCCA AGAAGGCGGC AACCGCATGA |
| Volvox | 49 | MTAMQANGGS GISDVTATAA TTGETSGGEA GPVGRLEAAT |
| carteri | | APTDAAALAY SDAGATGVDG LDETRKFVFA EEWGFSRVGG |
| f. nagariensis | | DLPDGTTPAT FADMFPERFF AFNPLRAVTA VALPLVAMAV |
| Glycolate | | GYGWLWYMHS ICPAWQQLAC AALIGTAYTG LFKVAHECAR |
| dehydrogenase | | FSFLPEAPRL QDALGLVLML PSLYPFTSWR LHYIHHMAHL |
| (XM_002946413) | | NMLWEDTFGW HPYTKLQLAT ELLCGQHWLL ALRRLALTTP |
| | | LKLFASVGHW LRSFDGLDLK RFHKDTYWAV LAGWSGPILF |
| | | LGFGIPAIIK MFGLSVRLAD RAWGGLVSGY LLPWLVFHFW |
| | | LSTLSLLQHT APHIPFRAED AGYDSGRAAI CGTVTVRLPR |
| | | PLELLLNDAN YSLPQLVAPG MPVFHAREAY EYMRERLLPY |
| | | LTEATLSVKL LTNHITKWQV YDAEKETYLP LDEVMQELER |
| | | DIATLMEEAE QAEEEEKVSG GPSGGAVEGQ GKRLAQLLGA |
| | | QLKQYISEVR SISSSNAAGR GALGSGHTPT TQVVTGQQTR |
| | | SHAQVAVQQA TQQHLKVVVP AIRTDLVGAV SMVSDGEKVE |
| | | PGVFKNIDGH RFDDGRYQAF IEEITKFIPK ERQYTDPVRT |
| | | FAYGIDASFY RLNPKLVVKV HNEEEVRKIL PIAERLKVPV |
| | | TFRAAGTSLS GQAITDSVLI KLSHTGKNFR NYTVHGDGSS |
| | | ITVEPGLIGG EVNRILAAYQ KKHKLPIQYK IGPDPSSIDS |
| | | CMIGGIVANN SSGMCCGVSQ NTYHTLKDMR VLFVDGTVLD |
| | | TADPDSCAAF LKSHKALVDG VVDLARRVQA DRELTALIRR |
| | | KFAIKCTTGY SLNALVDFPL DNPIEIIKHV IIGSEGTLGF |
| | | VSRATYNTVP EWPNKASAFI VFPDVRAACM GASVLRNETS |
| | | VDAVELFDRA SLRECENNED MMRLVPDIKG CDPMAAALLI |
| | | ECRGQDEAAL QGRIEEVVRA LTSAGLPVGA KAAEPRPITA |
| | | YPFHHDAKNS KVFWDVRRGL IPIVGAAREP GTSMLIEDVA |
| | | CPVDKLADMM IDLIDMFQRY GYNDASCFGH ALEGNLHLVF |
| | | SQGFRTKEEV QRFADMMEEM CYIVATKHSG SLKGEHGTGR |
| | | NVAPFVEMEW GSKAYELMWE LKELFDPKYT LNPGVILNRD |
| | | PDAHIKFLKP SPAASPIVNR CIECGFCESN CPSRDITLTP |
| | | RQRIAVYREM YRLKQLGPAA SEEEKQQLAA ITSSYAYDGE |
| | | QTCAADGMCQ EKCPVKINTG DMIKALRAEH MKEAKSASGA |
| | | AMWMANNFGV LNATVPRFLN LVNLAHSILG PKPLEAISRA |
| | | LNAATNHLVP VWNPYMPKGA APLKVPAPFP APAASSGGIP |
| | | RRVVYMPSCV TRMMGPAASD KETASVHEKI LSLFNKAGYE |
| | | VIVPEGVSSQ CCGMMFNSRG FKDAAAAKGA DLEAALLKAS |
| | | DNGKIPIVMD TSPCLAQVKS QISEPALRFA LYEPVEFIRH |
| | | FLVDKLEWRK VREQVAIHVP CSSKKMGIEE SFAKLAGLCA |
| | | HEVVPSGIPC CGMAGDRGMR YPELTGSSLQ HLNLPKSCSD |
| | | GYSTSRTCEM SLSNHSGINF RGLVYLVDEA TSPKKAATA |

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A transgenic plant cell comprising:
a first promoter operably linked to a polynucleotide sequence encoding a glycolate dehydrogenase;
a second promoter operably linked to a polynucleotide sequence encoding a malate synthase; and
an inhibitory polynucleotide targeting an endogenous glycolate transporter Plgg1, wherein expression of the endogenous glycolate transporter Plgg1 in the transgenic plant cell is about 20% to 80% of expression of endogenous glycolate transporter Plgg1 in a plant cell that is not transformed with an inhibitory polynucleotide targeting an endogenous glycolate transporter Plgg1.

2. The transgenic plant cell of claim 1, wherein the expression of the endogenous glycolate transporter Plgg1 is about 30% to 70% of expression of endogenous glycolate transporter Plgg1 in a plant cell that is not transformed with an inhibitory polynucleotide targeting an endogenous glycolate transporter Plgg1.

3. The transgenic plant cell of claim 1 or claim 2, wherein the first promoter is a constitutive promoter.

4. The transgenic plant cell of any one of claims 1-3, wherein the polynucleotide sequence encoding a glycolate dehydrogenase has an amino-terminus and is operably linked to a chloroplast-targeting signal sequence at the amino-terminus.

5. The transgenic plant cell of any one of claims 1-4, wherein the polynucleotide sequence encoding a malate synthase has an amino-terminus and is operably linked to a chloroplast-targeting signal sequence at the amino-terminus.

6. The transgenic plant cell of claim 4 or claim 5, wherein the chloroplast-targeting signal sequence encodes a ribulose-1,5-bisphosphate carboxylase-oxygenase (RuBisCO) small subunit RbcS signal sequence.

7. The transgenic plant cell of any one of claims 1-6, wherein the glycolate dehydrogenase is from an algae of the species Chlamydomonas reinhardtii.

8. The transgenic plant cell of any one of claims 1-7, wherein the malate synthase is from a plant of the genus Cucurbita.

9. The transgenic plant cell of any one of claims 1-8, wherein the inhibitory polynucleotide comprises a glycolate transporter Plgg1 polynucleotide sequence from a plant of the genus Populus, Eucalyptus, Pinus, Quercus, Pseudotsuga, Picea, Abies, Tsuga, Sequoia, Thuja, Chamaecyparis, or Ficus.

10. The transgenic plant cell of any one of claims 1-9, wherein the inhibitory polynucleotide comprises a glycolate transporter Plgg1 from a plant of the genus Populus.

11. The transgenic plant cell of any one of claims 1-10, wherein the inhibitory polynucleotide is an RNAi molecule comprising a sense strand of 20-400 contiguous nucleotides of a glycolate transporter Plgg1 of a plant of the genus Populus, and an antisense strand that hybridizes to the sense strand, wherein the sense and the antisense strands form a duplex.

12. The transgenic plant cell of any one of claims 1-11, wherein the inhibitory polynucleotide comprises a polynucleotide sequence of a glycolate transporter Plgg1 from a plant of the species Populus trichocarpa.

13. The transgenic plant cell of any one of claims 1-12, wherein the inhibitory polynucleotide is an RNAi molecule comprising a sense strand comprising at least 20 contiguous nucleotides of SEQ ID NO:6, and an antisense strand that hybridizes to the sense strand, wherein the sense and the antisense strands form a duplex.

14. The transgenic plant cell of any one of claims 1-13, wherein the transgenic plant cell is from a tree plant.

15. The transgenic plant cell of claim 14 wherein the tree plant of the genus Populus, Eucalyptus, Pinus, Quercus, Pseudotsuga, Picea, Abies, Tsuga, Sequoia, Thuja, Chamaecyparis, or Ficus.

16. The transgenic plant cell of claim 15, wherein the tree plant is of the genus Populus.

17. The transgenic plant cell of claim 14, wherein the tree plant is a hybrid Populus tremula x Populus alba.

18. A transgenic plant comprising the transgenic plant cell of any one of claims 1-17.

19. The transgenic plant of claim 18, wherein the transgenic plant comprises at least 10% more biomass accumulation compared to a non-transgenic plant of the same species.

20. The transgenic plant of 18 or claim 19, wherein the transgenic plant is a tree plant.

21. The transgenic plant of claim 20, wherein the tree plant is of the genus Populus, Eucalyptus, Pinus, Quercus, Pseudotsuga, Picea, Abies, Tsuga, Sequoia, Thuja, Chamaecyparis, or Ficus.

22. The transgenic plant of claim 20, wherein the tree plant is of the genus Populus.

23. The transgenic plant of claim 20, wherein the tree plant is a hybrid of Populus tremula x Populus alba.

24. A method for increasing biomass productivity in a plant, said method comprising:
transforming a plant cell with one or more polynucleotides comprising:
a first promoter operably linked to a polynucleotide sequence encoding a glycolate dehydrogenase;
a second promoter operably linked to a polynucleotide sequence encoding a malate synthase; and
an inhibitory polynucleotide targeting an endogenous glycolate transporter Plgg1, wherein expression of the endogenous glycolate transporter Plgg1 in the transformed plant cell is about 20% to 80% of expression of endogenous glycolate transporter Plgg1 in a plant cell that is not transformed with an inhibitory polynucleotide targeting an endogenous glycolate transporter Plgg1, wherein said transforming increases biomass productivity of the plant.

25. The method of claim 24, wherein the expression of the endogenous glycolate transporter Plgg1 is about 30% to 70% of expression of endogenous glycolate transporter Plgg1 in a plant cell that is not transformed with an inhibitory polynucleotide targeting an endogenous glycolate transporter Plgg1.

26. The method of claim 24 or claim 25, wherein the inhibitory polynucleotide targets a glycolate transporter Plgg1 from a plant of the genus Populus, Eucalyptus, Pinus, Quercus, Pseudotsuga, Picea, or Ficus.

27. The method of any one of claims 24-26, wherein the inhibitory polynucleotide targets glycolate transporter Plgg1 from a plant of the genus Populus.

28. The method of any one of claims 24-27, wherein the inhibitory polynucleotide is an RNAi molecule comprising a sense strand of 20-400 contiguous nucleotides of a glycolate transporter Plgg1 of a plant of the genus Populus, and an antisense strand that hybridizes to the sense strand, wherein the sense and the antisense strands form a duplex.

29. The method of any one of claims 24-28, wherein the inhibitory polynucleotide targets a glycolate transporter Plgg1 from a plant of the species Populus trichocarpa.

30. The method of any one of claims 24-29, wherein the inhibitory polynucleotide is an RNAi molecule comprising a sense strand comprising at least 20 contiguous nucleotides of SEQ ID NO:6, and an antisense strand that hybridizes to the sense strand, wherein the sense and the antisense strands form a duplex.

31. The method of any one of claims 24-30, wherein the inhibitory polynucleotide further comprises an intron polynucleotide.

32. The method of any one of claims 24-31, wherein said transforming is carried out by introducing the one or more polynucleotides into the plant cell by Agrobacterium tumefaciens infection, electroporation, or particle bombardment to obtain transgenic plant cells.

33. A transgenic plant cell culture produced by the method of claim 32.

34. The method of claim 32 further comprising:
culturing the transgenic plant cell.

35. The method of any one of claim 24-32 or 34 further comprising:
regenerating a transgenic plant from the transgenic plant cell.

36. The method of claim 35, wherein the transgenic plant is a tree plant.

37. The method of claim 35 or claim 36, wherein the transgenic plant shows enhanced biomass productivity compared to a non-transgenic plant of the same species.

38. The method of claim 36, wherein the tree plant is of the genus Populus, Eucalyptus, Pinus, Quercus, Pseudotsuga, Picea, Abies, Tsuga, Sequoia, Thuja, Chamaecyparis, or Ficus.

39. The method of claim 38, wherein the tree plant is of the genus Populus.

40. The method of claim 39, wherein the tree plant is a hybrid Populus tremula x Populus alba.

41. A method for increasing photosynthetic activity and stomatal conductance under drought conditions in a plant, said method comprising:
transforming a plant cell with one or more polynucleotides comprising:
a first promoter operably linked to a polynucleotide sequence encoding a glycolate dehydrogenase;
a second promoter operably linked to a polynucleotide sequence encoding a malate synthase; and
an inhibitory polynucleotide targeting an endogenous glycolate transporter Plgg1, wherein expression of the endogenous glycolate transporter Plgg1 in the transgenic plant cell is about 20% to 80% of expression of endogenous glycolate transporter Plgg1 in a plant cell that is not transformed with an inhibitory polynucleotide targeting an endogenous glycolate transporter Plgg1;
regenerating a plant from the transformed plant cell; and
subjecting the plant to drought conditions, wherein said transforming increases photosynthetic activity and stomatal conductance under drought conditions in the regenerated plant.

42. A polynucleotide construct comprising:
an inhibitory polynucleotide comprising a polynucleotide sequence that targets an endogenous glycolate transporter Plgg1 present in a plant cell, wherein when expressed in the plant cell, the inhibitory polynucleotide reduces expression of the endogenous glycolate transporter Plgg1 by about 20% to 80% of expression of endogenous glycolate transporter Plgg1 in a plant cell where the inhibitory polynucleotide is not present.

43. The polynucleotide construct according to claim 42 further comprising:
a first polynucleotide sequence encoding a glycolate dehydrogenase; and
a first promoter operably linked to the first polynucleotide sequence;

44. The polynucleotide construct according to claim 42 or claim 43 further comprising:
a second polynucleotide sequence encoding a malate synthase; and
a second promoter operably linked to the second polynucleotide sequence.


SEQUENCE LISTING (49 sequences)

SEQ ID NO: 1 - DNA, 3288 bp, Chlamydomonas reinhardtii
  source 1..3288: mol_type = unassigned DNA
  misc_feature 1..3288: note = glycolate dehydrogenase
atgccacgcggccagggcaagcgcctggctcagctccttggagctcagctgaagcagtacgcagcggaggtgcgtggcatcagcacagctggtggcgcttctcgcggtggagctcgaggacctgcatcccctagctcgctagagcagcagacgcgccaggtcgctcaggttgctgttcagcagtcgactcagcaggcagtgaaggtcgttgtgccggccatcaaagtagacctggttggtgcggtcagctcggtgtctgagagcgacaaggtggagccgggtgtgttcaagaacgtggatggccaccgcttcgaggacggtcgctatgccgcttttgttgaggagattacaaagtttatccccaaggagcgccagtactcggaccccgtgcgcacattcgcgtatggcacggatgcctccttctaccggcttaacccgaagctggtagtgaaggtgcacaacgaggacgaggtccgccgcatcatgcccatcgcggagcggctgcaggtccctatcaccttccgcgcggccggcacgtcgctgtctgggcaggcaattaccgactcggtgctcattaagctgagccacacgggcaagaacttccgcaactttaccgtgcacggcgacggtagcgtgatcacggtggagccgggcctcattggcggcgaggtgaaccgcatcctggcggcacaccagaagaagaacaagctgcccatccagtacaagatcggacccgacccctcctccatcgacagctgcatgatcggcggcatcgtgtccaacaacagcagcggcatgtgctgcggcgtgagccagaacacctaccacacgctgaaggacatgcgggtggtgttcgtagacggaacggtgctggacacggccgaccccaactcgtgcaccgccttcatgaagagccaccgctcgctggtggatggcgtcgtgagcctggcgcgccgcgtgcaggccgacaaggagctgacggcgctcatccgccgcaagttcgccatcaagtgcaccaccggctactccctgaacgcgctggtggacttcccggtggacaaccccattgagatcatcaagcacctcatcatcggcagcgagggcacgctgggcttcgtcagccgcgccacctacaacaccgtgcccgagtggcccaacaaggcctcggccttcatcgtgttcccggacgtgcgcgccgcctgcaccggcgcctcggtgctgcgcaacgagacgtccgtggacgcggtggagctgtttgaccgcgccagcctgcgcgagtgcgagaacaacgaggacatgatgcgcctggtgcccgacatcaagggctgcgaccccatggcggcagcgctgctgatcgagtgccgcggccaggacgaggccgcactgcagagccgcattgaggaggtggtgcgcgtgctgacggcggcgggcctgcccttcggcgccaaggccgcgcagcccatggccatcgacgcctaccccttccaccacgaccagaagaacgccaaggtcttctgggacgtgcgcaggggcctgatccccattgtgggcgcggcgcgcgagcccggcacatccatgctgatcgaggacgtggcctgccccgtggacaagctggccgacatgatgatcgacctgatcgacatgttccagcgccacggctaccacgacgcctcctgcttcggccacgcgctcgagggcaaccttcacttggtgttctcgcagggcttccgcaacaaggaggaggtgcagcgcttcagcgacatgatggaggagatgtgccacctggtggccaccaagcactcgggcagcctcaagggcgagcacggcacgggccgcaacgtggcgccgttcgtggagatggagtggggcaacaaggcgtacgagctgatgtgggagctcaaggcgctgttcgaccccagccacaccctcaacccgggcgtcatcctcaaccgcgaccaggacgcgcacatcaagttcctgaagccctcgcccgcggcctcgcccatcgtcaaccgctgcatcgagtgcggcttctgcgagtccaactgcccctcgcgcgacatcacgctcacgccgcgccagcgcatctccgtgtaccgcgagatgtaccgcctcaagcagctgggcccgggcgccagcgaggaggagaagaagcagctggcggccatgagcagctcgtacgcctacgacggcgagcagacgtgcgcggcggacggcatgtgccaggagaagtgccccgtcaagatcaacacgggagacctgatcaagtcgatgcgtgccgagcacatgaaggaggagaagaccgccagcggcatggcagactggctggccgccaacttcggcgtcatcaactccaacgtgccgcgcttcctcaacatcgtcaacgccatgcacagcgtggtgggctcggcgcctctgtccgccatcagccgcgcgctcaacgccgccaccaaccacttcgtgccggtgtggaacccctacatgcccaagggcgcggcgccgctcaaggtgcccgccccgccggcgccggcagctgctgaggcctcgggcatcccgcgcaaggtggtgtacatgcccagctgcgtgacgcgcatgatgggccccgccgcctccgacaccgagacggcggcggtgcacgagaaggtgatgagcctgttcggcaaggccggctacgaggtgatcatccccgagggcgtggccagccagtgctgcggcatgatgttcaacagccgcggcttcaaggacgccgccgccagcaagggcgcggagctggaggcggcgctgctcaaggcctcggacaatggcaagatccccatcgtcatcgacacctcgccctgcctggcgcaggtgaagagccagatcagcgagccgtcgctgcgcttcgcgctgtacgagccggttgagttcatccggcacttcctggtggacaagctggagtggaagaaggtgcgcgaccaggtggccatccacgtgccctgctcctccaagaagatgggcatcgaggagtccttcgcgaagctggcgggcctgtgcgccaacgaggtggtgccctcgggcattccttgctgcggcatggcgggcgaccgcggcatgcgcttccccgagctgaccggcgcctcgctgcagcacctcaacctgcccaagacctgcaaggacggctactccaccagccgcacctgcgagatgtcgctcagcaaccacgccggcatcaacttcaggggcctggtgtacctggtggatgaggccacggcgcctaagaagcaggccgccgctgccaagaccgcgtaa

SEQ ID NO: 2 - protein, 1095 aa, Chlamydomonas reinhardtii
  REGION 1..1095: note = glycolate dehydrogenase
MPRGQGKRLAQLLGAQLKQYAAEVRGISTAGGASRGGARGPASPSSLEQQTRQVAQVAVQQSTQQAVKVVVPAIKVDLVGAVSSVSESDKVEPGVFKNVDGHRFEDGRYAAFVEEITKFIPKERQYSDPVRTFAYGTDASFYRLNPKLVVKVHNEDEVRRIMPIAERLQVPITFRAAGTSLSGQAITDSVLIKLSHTGKNFRNFTVHGDGSVITVEPGLIGGEVNRILAAHQKKNKLPIQYKIGPDPSSIDSCMIGGIVSNNSSGMCCGVSQNTYHTLKDMRVVFVDGTVLDTADPNSCTAFMKSHRSLVDGVVSLARRVQADKELTALIRRKFAIKCTTGYSLNALVDFPVDNPIEIIKHLIIGSEGTLGFVSRATYNTVPEWPNKASAFIVFPDVRAACTGASVLRNETSVDAVELFDRASLRECENNEDMMRLVPDIKGCDPMAAALLIECRGQDEAALQSRIEEVVRVLTAAGLPFGAKAAQPMAIDAYPFHHDQKNAKVFWDVRRGLIPIVGAAREPGTSMLIEDVACPVDKLADMMIDLIDMFQRHGYHDASCFGHALEGNLHLVFSQGFRNKEEVQRFSDMMEEMCHLVATKHSGSLKGEHGTGRNVAPFVEMEWGNKAYELMWELKALFDPSHTLNPGVILNRDQDAHIKFLKPSPAASPIVNRCIECGFCESNCPSRDITLTPRQRISVYREMYRLKQLGPGASEEEKKQLAAMSSSYAYDGEQTCAADGMCQEKCPVKINTGDLIKSMRAEHMKEEKTASGMADWLAANFGVINSNVPRFLNIVNAMHSVVGSAPLSAISRALNAATNHFVPVWNPYMPKGAAPLKVPAPPAPAAAEASGIPRKVVYMPSCVTRMMGPAASDTETAAVHEKVMSLFGKAGYEVIIPEGVASQCCGMMFNSRGFKDAAASKGAELEAALLKASDNGKIPIVIDTSPCLAQVKSQISEPSLRFALYEPVEFIRHFLVDKLEWKKVRDQVAIHVPCSSKKMGIEESFAKLAGLCANEVVPSGIPCCGMAGDRGMRFPELTGASLQHLNLPKTCKDGYSTSRTCEMSLSNHAGINFRGLVYLVDEATAPKKQAAAAKTA

SEQ ID NO: 3 - DNA, 1701 bp, Cucurbita hybrida
  source 1..1701: mol_type = unassigned DNA
  misc_feature 1..1701: note = malate synthase
atgggatcgctgggaatgtattctgaatcggcagtaaggaagaaaagtagccgaggctacgatgttccagagggagtggacattcggggacgttatgatgaagaatttgccaggattctcaacaaggaagccttgctgtttgtggctgatttacagaggactttcagaaaccacataaggtattcgatggaatgccgcagagaagccaaaaggaggtacaatgaaggggcggtgccggggtttgatccggcgaccaagtatataagggaatctgagtggacatgtgcatcagtccccccggcagttgctgatcggagagtggagatcaccggacctgtggagcggaagatgatcatcaacgcactcaattctggagctaaagttttcatggcggactttgaagatgcactatcaccaaactgggagaatttgatgagggggcaaattaatctgaaggatgcagttgatgggactataagcttccatgacaaagctagaaacaaggtttataaactgaacgatcagacagccaagctctttgttcgccctcgaggttggcacttcgctgaggctcatatcttcatcgacggcgagcctgccaccggctgtcttgtggacttcgggctctacttttttcacaaccatgccaatttccggcgctctcaaggtcaaggttctggccctttcttttaccttcccaaaatggagcactccagggaagcaaaaatatggaacagtgtatttgagagagcagagaagatggcagggatagagaggggcagcatcagggccactgtgctgattgaaacacttccagcagtgtttcaaatggatgaaatactctatgagctgagggatcattctgtgggattgaactgtggtagatgggattacatattcagctatgtcaagaccttccaggctcacctagatcgcctgttacccgaccgagtccaagtcggtatggcacaacatttcatgaggagttattctgatctccttatcaggacttgtcacacggtggtgtgccacgtgggaggcatggctgctcaaattccaattagagacgacccgaaggcaaatgagatggcacttgagctagtgaggaaggacaaattgagagaggcaaaggcaggacatgatggaacatgggcagcacatccaggattaatcccagcatgtatggaagtcttcaccaacagcatgggaaatgcccccaatcagatccgatctgcaagacgagacgatgctgcaaacctaactgaagacgacctcttgcagcaaccgaggggtgttcgtacattggaagggctccggttgaacacccgagtcggaattcagtacctagcagcatggctaaccgggacaggctctgtgcctctctacaaccttatggaagatgcagccacagctgaaatcagcagggttcaaaactggcaatggctgaagtatggagtggaattggatggagatgggcttggagtgagagtgaacaaggaactgttcgcaagagtggtggaagaagaaatggaaaggattgaaagagaagtggggaaggagaaattcaggaagggaatgtacaaagaggcttgcaagatgttcacaaggcaatgcacagcgccaaccttggatgattttctgaccttggatgcgtacaaccacatagtcatacatcatcccagggagctgtccaggctctga

SEQ ID NO: 4 - protein, 566 aa, Cucurbita hybrida
  REGION 1..566: note = malate synthase
MGSLGMYSESAVRKKSSRGYDVPEGVDIRGRYDEEFARILNKEALLFVADLQRTFRNHIRYSMECRREAKRRYNEGAVPGFDPATKYIRESEWTCASVPPAVADRRVEITGPVERKMIINALNSGAKVFMADFEDALSPNWENLMRGQINLKDAVDGTISFHDKARNKVYKLNDQTAKLFVRPRGWHFAEAHIFIDGEPATGCLVDFGLYFFHNHANFRRSQGQGSGPFFYLPKMEHSREAKIWNSVFERAEKMAGIERGSIRATVLIETLPAVFQMDEILYELRDHSVGLNCGRWDYIFSYVKTFQAHLDRLLPDRVQVGMAQHFMRSYSDLLIRTCHTVVCHVGGMAAQIPIRDDPKANEMALELVRKDKLREAKAGHDGTWAAHPGLIPACMEVFTNSMGNAPNQIRSARRDDAANLTEDDLLQQPRGVRTLEGLRLNTRVGIQYLAAWLTGTGSVPLYNLMEDAATAEISRVQNWQWLKYGVELDGDGLGVRVNKELFARVVEEEMERIEREVGKEKFRKGMYKEACKMFTRQCTAPTLDDFLTLDAYNHIVIHHPRELSRL

SEQ ID NO: 5 - DNA, 6210 bp, Populus trichocarpa
  source 1..6210: mol_type = genomic DNA
  misc_feature 1..6210: note = glycolate transporter Plgg1
atggctactcctttagtcgctcattccgttcagctctgtcatcaccactcaaaacaatattctttcaaatcacaatcacgtgtcaatagggatatcggtacaaaaacactacttggtgtttacaatggattccacaatgtatataaccaatcttattttcataagccttgggcacccattagagttcttgagcctaattcaaggttcttgcaaatgggtcctcaagaaacctgctctagtcgaggaatttccaagaaatctatgagctcagaaggcagtaccagtactagctcttcatctatttctcaacaggttagtataaaggttttactgtttttcttttttgcactcctttttatttttttttatgctttcctgttttgtatgatttctactaattggtattgagttgttgcatttaattaagctgattcaactatttgatttacctcctttttatattgattgcataaggtgattgggatcttgcatttgcttgtttcacttgggattatccttgcaatggataagttgttgaaaaaggcatttgtggctgctgctatcaagtttccaagtgctctgtttggcatgttctgcatattctcagttttagtcattcttgatataactattccggctgctgcaacaagcttaatgaacttctttcagccagcactgttattcattcagagatggcttccgttgttctacgttccatcattggttgttttgcctctctctgttaaagatatccctgctgcatcaggtgtcaagatttgcttcatcacaggtaaaacgaagctttttgttgattcattgcttttcactctctaaatctttaatatagttggcttgatgaataaaatgtcactatgaatttgtgataatcacacataattaatgttatctattgaaatcacagaaattactgcaagctcaaaagccaagataacatagcatctccttcattctccttatgtttcaagttttttgctgaccatttcattattcatatctccgattccaatggccaccattttttttctcactttggtcattattcatataccatccagtcagcattcccatgtttacttccattttggtgggactatttttcaagttgtgttttgcaattgtaataaataataataataatgatgatacaaatgctttctaataactgcagctggagggtggttggcgtcactttgtgtggcgggttttacagctattgctgtgagaaaaatggtgaagacagaaatgactgatgctgagcctatggcgaaaccctctcctttttctccattggaaatatgggcttggagtggggtcttccttgtatcatttgttgtcgcattattatatagaacggcattagggactgcagccagaacatgccttcctttcctactagcgtccaccgtgctaggctatatggttggttctgggtaaaatctcagtttcctcagattagcaacctctgtgttgacaattgttttccagaccaaattgacgaggaatggaattgttgcacacattcattataattgtagcccagttaaaaaatcatggctttactcttctggttttcaggttaccatctggtgttaagaaggttttccatcccattatttgttgtgcactatctgcagatttggcagcgttggcctttgggttcctttcccaatctggactcgatcccgttctaggtaccacttttcaaagtgtttgctgtcttcatttagttcactcattttctgatgtggagcaacaacaaatgacaacaattgttactacaaaatgcctgtattattttgttccacaagccatctcttgcttttcctttctatcatatgtgttagtttccattatgtattatgtttccttctgatacatgtagtgttagtagagaacaaaacctggaggggccatttcaggcagtttccattgacaaacttaggtagtgtttggtattccgaacaaagatggaccggatatgacaatattttttgtgaagttgttttgtgtacttttgaatagtacaaaaatcaatttttatcttgtccaaagctgttctagtagagaaaattgtctcattagaaggggtagaacaaccttgtctagttccttgcagaagagttaacttaggttgatccgaatcagcataaaataaaaatagttattgttatagttatagttttaaacttgactagagagttgactcagggcaaagtttgggttacggattaggttgactattgactcaagtcaatgtaaggataaaaatgtttattatcgtagttttaaaacttgacttagggtcgaaccggggcaaagccccgatcacgggtcgggttgactattgatctaggtcaatgtaaggataaaattattgttatcatagctttaaaacctgactagggggttgacccgggtcacgggttggttgaccattgagccgagtcaacgtaaagataaaaatgattattatcacagttttaaaacctgatttaggagttgacctgggataagacctaagtcacaggttgagatgattattgaacaaggtcaatgtaaagataaaagtgtttattattatagttttaaagtctaactaggaggtcaactcgggtaaagtccctgtcacaggtttggttaaccattgacccaggtcaatataaagataaaagtagcttgagttacaagtcgggttgaccattgatctgagttaatgtaaggataaaaatagttattatcatagtattaaaactcgactaggagatcgacctgaggtaagacccatgtcacgggtcgagatggtcaacttgttttgacccaagataacgtaagagtaagaataattattatcaaaattttaaaatccgactcgaggttcagcttgagtcatgggttgggagggttaacgtaagaataaaaatgattattattataattttaaaactcaatttaaaggtcgatccggggcaaggcctgagttatgggttgagatggtcagcttgggttgacttaggtcaatatatagataaaaatagttattatcataattttaaaacctaacttgggggtcgacctggggcaaggtttgagtcttaagatgagagggtcaacctaagttgacaaaatattttgttttttaaaaaaatcaaaacaattttgttttataaaaaaaaataaaaaaagtcaaaaggatttcttagccttgttttatcttgggtcgcaagtcgacctgattttttaaccgagtagggtcggatcaattgtttctttattttttctcaaacttagattagtccaattcttgtgtcagtcgggtctcatccggtatctaccaaacacgaaataaaataaattgtctagttcaattcaaagcacactaaatacaaaaacaaaacaattatttgtcgagtccaattaatattgtctagcataccaaacactaccctaccactcatagcctttgtgcaaaatgttgagggacctcgaacgttttatttattgcatcacttgtgctaatactgttttgtcaatgataaaaaagatggttatttatgatctaatgatgaacacttctataattcaaaactgaagttaactgatatatagttttacaaatattttggacaacattttttaaatttcagggatgaaaatcttaaagttcaaaagccgttttgatctagtttctgttggaattcaaatcacataccatgtgaactccctcttttttcctccatccttctctatggtcacatttttttcattaagttttttcatatctatttttaagctcattttctgtacttcctcattgtagttatggtaattttcttccaatgcacacaggatattaccttacaaaagtttcatctaatcctggagctggtgatgtgttaatgggattcttgggacctgtcattctttcttttgccttctcaatgttcaagcagcgaaaggtaacttcttttctcacaatccatgccaacaggtcgggttcattttgtattaagtggatatgtttcatctgtgctgttactcaggaaggactagcggttccttttatttaattgtaactatttggtgttgccaaccaaactatctggtgttgccaaccaaactatctgctatacattattttttttcatttggtaatggaagacgatgcgtttgaaattcagagtagagaataagcaatgaaaaacctcaaaatatttagaccttgcaagaatctaggaatctagaatattgcatctacctttatatgaatttatcctcagtagtattctatgtatgctttgtcattacatggtttgttgtcctcgattcagaataataaaaatttgtcatcagatgatgctcttaacttactggtccttttgtcatggcaatgtgagtttatatactctggtgtttctgcagcttgttaagagacatgcagctgagattttcacgtccgtcattgttgcaacactattttcattgtattcaacggctctcgtgggacgtctagttgggttagaaccaacgttgactgtatccattattcccagatgtataaccgtggcattagccctcagcattgtgtcattctttgaaggtatagtaatgttcagaagccagctggaagcagggaattccataaatgggcatccactagtgatgctaaaattaaattttgacatatcaaacaccgttattctgagatgaattcatttccaatttacttaacgactttggtcagttagaacacaatgtactgcagatgattggtgtgactcattaccatttttaatcgaattaattttacatattggttgtaggtgccaattcatctctcacagctgctgtagttgttgtaactggtctcattggagcaaattttgtacaagcagtgctcgataaattaaattttcgtgatcccattgctcgaggaatagcaactgcttccaggtttgtggtttaccaaagacaaatcagtacgaactcaagcactaaacaatctttgcaagcatagacccttccctttctttagtaaatataatgatgtgtcatatcagctacaagcaactatctttctcatagctcacctgtatagttttctttcaggggactgcaacaaaaatgcatctgggaccaatactttgtactaacaaaatgcttgctgttggccagaaactggaaactgaatctcctggggaacttaaaactggttaataattaagctatttaatatgtagaaaattctgaatgtcactgagattcttattgctgaatcttataaaaatcaaaatcattttagaattggactagtcaatgaaaggagatctgtgtttttctgttttggttgaatattttagtcattttggttatagcatataaaaacaaaaccgtggattgaaaataagaagaaagataggatgctcgaactttgctcactgaattggacttgatgcacaatcttgagttatgctttaaaaaccttacccaagtttgtttgtgacttcggttatagtcgcagcttgtgatgctcagataagctcagtttacctgttctgttcaagagtgtacaacctgacatgctcagtcctgagtattatattcatatcctactttagttttcctgtgtgaagattttgcaaagttttgtaggaactgtgtcccattactgcatttatgcttatccttcttaattgtttcagtgctcatggactgggaactgcagcattgtcagccaaggaacctgaggcacttccattttgtgccattgcttatgctctcactggtatatttggttcattgttttgttcagttcctgcagttaggcaaagcttacttgcaataattggctgaaagattgggcagcttttggttattggtggccagacacacttttgattgattatccttgtccacccacagcatgatttttagcaagtacacagtgctaatagccaaccttgtattattgattacagtcaattatgtatagaagttgacatcatccttctgcaaaaactcctcacctcctttaaattcctttaaattatgaatagtgaactgattgtcttcttgttagaaggcggtcagctggctgaaaagcaaaagctaaaggtcaaaggtaaattatctattctacaccttgagagggtggaaaatccagtggataccaaagaagccactgttgctcag

SEQ ID NO: 6 - DNA, 1575 bp, Populus trichocarpa
  source 1..1575: mol_type = unassigned DNA
  misc_feature 1..1575: note = PtPlgg1
atggctactcctttagtcgctcattccgttcagctctgtcatcaccactcaaaacaatattctttcaaatcacaatcacgtgtcaatagggatatcggtacaaaaacactacttggtgtttacaatggattccacaatgtatataaccaatcttattttcataagccttgggcacccattagagttcttgagcctaattcaaggttcttgcaaatgggtcctcaagaaacctgctctagtcgaggaatttccaagaaatctatgagctcagaaggcagtaccagtactagctcttcatctatttctcaacaggtgattgggatcttgcatttgcttgtttcacttgggattatccttgcaatggataagttgttgaaaaaggcatttgtggctgctgctatcaagtttccaagtgctctgtttggcatgttctgcatattctcagttttagtcattcttgatataactattccggctgctgcaacaagcttaatgaacttctttcagccagcactgttattcattcagagatggcttccgttgttctacgttccatcattggttgttttgcctctctctgttaaagatatccctgctgcatcaggtgtcaagatttgcttcatcacagctggagggtggttggcgtcactttgtgtggcgggttttacagctattgctgtgagaaaaatggtgaagacagaaatgactgatgctgagcctatggcgaaaccctctcctttttctccattggaaatatgggcttggagtggggtcttccttgtatcatttgttgtcgcattattatatagaacggcattagggactgcagccagaacatgccttcctttcctactagcgtccaccgtgctaggctatatggttggttctgggttaccatctggtgttaagaaggttttccatcccattatttgttgtgcactatctgcagatttggcagcgttggcctttgggttcctttcccaatctggactcgatcccgttctaggatattaccttacaaaagtttcatctaatcctggagctggtgatgtgttaatgggattcttgggacctgtcattctttcttttgccttctcaatgttcaagcagcgaaagcttgttaagagacatgcagctgagattttcacgtccgtcattgttgcaacactattttcattgtattcaacggctctcgtgggacgtctagttgggttagaaccaacgttgactgtatccattattcccagatgtataaccgtggcattagccctcagcattgtgtcattctttgaaggtgccaattcatctctcacagctgctgtagttgttgtaactggtctcattggagcaaattttgtacaagcagtgctcgataaattaaattttcgtgatcccattgctcgaggaatagcaactgcttccagtgctcatggactgggaactgcagcattgtcagccaaggaacctgaggcacttccattttgtgccattgcttatgctctcactggtatatttggttcattgttttgttcagttcctgcagttaggcaaagcttacttgcaataattggctga

SEQ ID NO: 7 - protein, 524 aa, Populus trichocarpa
  REGION 1..524: note = PtPlgg1
MATPLVAHSVQLCHHHSKQYSFKSQSRVNRDIGTKTLLGVYNGFHNVYNQSYFHKPWAPIRVLEPNSRFLQMGPQETCSSRGISKKSMSSEGSTSTSSSSISQQVIGILHLLVSLGIILAMDKLLKKAFVAAAIKFPSALFGMFCIFSVLVILDITIPAAATSLMNFFQPALLFIQRWLPLFYVPSLVVLPLSVKDIPAASGVKICFITAGGWLASLCVAGFTAIAVRKMVKTEMTDAEPMAKPSPFSPLEIWAWSGVFLVSFVVALLYRTALGTAARTCLPFLLASTVLGYMVGSGLPSGVKKVFHPIICCALSADLAALAFGFLSQSGLDPVLGYYLTKVSSNPGAGDVLMGFLGPVILSFAFSMFKQRKLVKRHAAEIFTSVIVATLFSLYSTALVGRLVGLEPTLTVSIIPRCITVALALSIVSFFEGANSSLTAAVVVVTGLIGANFVQAVLDKLNFRDPIARGIATASSAHGLGTAALSAKEPEALPFCAIAYALTGIFGSLFCSVPAVRQSLLAIIG

SEQ ID NO: 8 - DNA, 257 bp, synthetic construct
  source 1..257: mol_type = other DNA
  misc_feature 1..257: note = PtPlgg1 sense direction
ggaaatatgggcttggagtggggtcttccttgtatcatttgttgtcgcattattatatagaacggcattagggactgcagccagaacatgccttcctttcctactagcgtccaccgtgctaggctatatggttggttctgggttaccatctggtgttaagaaggttttccatcccattatttgttgtgcactatctgcagatttggcagcgttggcctttgggttcctttcccaatctggactcgatcccgttctag

SEQ ID NO: 9 - DNA, 257 bp, synthetic construct
  source 1..257: mol_type = other DNA
  misc_feature 1..257: note = PtPlgg1 antisense direction
ctagaacgggatcgagtccagattgggaaaggaacccaaaggccaacgctgccaaatctgcagatagtgcacaacaaataatgggatggaaaaccttcttaacaccagatggtaacccagaaccaaccatatagcctagcacggtggacgctagtaggaaaggaaggcatgttctggctgcagtccctaatgccgttctatataataatgcgacaacaaatgatacaaggaagaccccactccaagcccatatttcc

SEQ ID NO: 10 - DNA, 23 bp, synthetic construct
  source 1..23: mol_type = other DNA
  misc_feature 1..23: note = Plgg1 primer
caggtgtcaagatttgcttcatc

SEQ ID NO: 11 - DNA, 19 bp, synthetic construct
  source 1..19: mol_type = other DNA
  misc_feature 1..19: note = Plgg1 primer
actccaagcccacatttcc

SEQ ID NO: 12 - DNA, 21 bp, synthetic construct
  source 1..21: mol_type = other DNA
  misc_feature 1..21: note = PtACT2 primer
actcacaccatcaccagaatc

SEQ ID NO: 13 - DNA, 22 bp, synthetic construct
  source 1..22: mol_type = other DNA
  misc_feature 1..22: note = PtACT2 primer
ctcaaccctaaggctaacagag

SEQ ID NO: 14 - DNA, 25 bp, synthetic construct
  source 1..25: mol_type = other DNA
  misc_feature 1..25: note = ChMS primer
ccacataaggtattcgatggaatgc

SEQ ID NO: 15 - DNA, 21 bp, synthetic construct
  source 1..21: mol_type = other DNA
  misc_feature 1..21: note = ChMS primer
gactgatgcacatgtccactc

SEQ ID NO: 16 - DNA, 23 bp, synthetic construct
  source 1..23: mol_type = other DNA
  misc_feature 1..23: note = CrGDH primer
cacatcaagttcctgaagccctc

SEQ ID NO: 17 - DNA, 21 bp, synthetic construct
  source 1..21: mol_type = other DNA
  misc_feature 1..21: note = CrGDH primer
atctcgcggtacacggagatg

SEQ ID NO: 18 - DNA, 21 bp, synthetic construct
  source 1..21: mol_type = other DNA
  misc_feature 1..21: note = nptII primer
cctgccgagaaagtatccatc

SEQ ID NO: 19 - DNA, 20 bp, synthetic construct
  source 1..20: mol_type = other DNA
  misc_feature 1..20: note = nptII primer
catcctgatcgacaagaccg

SEQ ID NO: 20 - DNA, 23 bp, synthetic construct
  source 1..23: mol_type = other DNA
  misc_feature 1..23: note = Plgg1 primer
caggtgtcaagatttgcttcatc

SEQ ID NO: 21 - DNA, 22 bp, synthetic construct
  source 1..22: mol_type = other DNA
  misc_feature 1..22: note = Plgg1 probe
ctttgcgtggcgggtttcacag

SEQ ID NO: 22 - DNA, 21 bp, synthetic construct
  source 1..21: mol_type = other DNA
  misc_feature 1..21: note = Plgg1 primer
agcgtcagtcatttctgtctt

SEQ ID NO: 23 - DNA, 21 bp, synthetic construct
  source 1..21: mol_type = other DNA
  misc_feature 1..21: note = PtACT2 primer
ctgcaatgtatgttgccatcc

SEQ ID NO: 24 - DNA, 24 bp, synthetic construct
  source 1..24: mol_type = other DNA
  misc_feature 1..24: note = PtACT2 probe
actggcatacagggaaaggacagc

SEQ ID NO: 25 - DNA, 20 bp, synthetic construct
  source 1..20: mol_type = other DNA
  misc_feature 1..20: note = PtACT2 primer
cacaccatcaccagaatcca

SEQ ID NO: 26 - DNA, 21 bp, synthetic construct
  source 1..21: mol_type = other DNA
  misc_feature 1..21: note = PtEF1B-1 primer
catcactaccactccacacaa

SEQ ID NO: 27 - DNA, 30 bp, synthetic construct
  source 1..30: mol_type = other DNA
  misc_feature 1..30: note = PtEF1B-1 probe
tcctctttcaatcacactgtttatcgctct

SEQ ID NO: 28 - DNA, 25 bp, synthetic construct
  source 1..25: mol_type = other DNA
  misc_feature 1..25: note = PtEF1B-1 primer
tttcaattacttggaagatgggatg

SEQ ID NO: 29 - DNA, 18 bp, synthetic construct
  source 1..18: mol_type = other DNA
  misc_feature 1..18: note = CrGDH primer
aaggcgtacgagctgatg

SEQ ID NO: 30 - DNA, 20 bp, synthetic construct
  source 1..20: mol_type = other DNA
  misc_feature 1..20: note = CrGDH probe
aacccgggcgtcatcctcaa

SEQ ID NO: 31 - DNA, 20 bp, synthetic construct
  source 1..20: mol_type = other DNA
  misc_feature 1..20: note = CrGDH primer
gagggcttcaggaacttgat

SEQ ID NO: 32 - DNA, 19 bp, synthetic construct
  source 1..19: mol_type = other DNA
  misc_feature 1..19: note = ChMS primer
atggaatgccgcagagaag

SEQ ID NO: 33 - DNA, 24 bp, synthetic construct
  source 1..24: mol_type = other DNA
  misc_feature 1..24: note = ChMS probe
atacttggtcgccggatcaaaccc

SEQ ID NO: 34 - DNA, 21 bp, synthetic construct
  source 1..21: mol_type = other DNA
  misc_feature 1..21: note = ChMS primer
gatgcacatgtccactcagat

SEQ ID NO: 35 - DNA, 1344 bp, Pinus taeda
  source 1..1344: mol_type = unassigned DNA
  misc_feature 1..1344: note = Plgg1
atggcttcatcccaccaattccaaacttcatggctgctcacacggagaccgttgggagacgcctctttgcgtttatatcaattaccccgcacgaccacaaaagagcagaggaaaaattatgacaattgcagctgttgggcgcagaggagaagagaggcagctgtcgctgcagcaatcagaattaatggtgagcgtaagttcatgttcacggggtttggcaggcaaaggccaaggcgaagtcatgaaataataatggtgaaagcctttgaaaaggatgccagaggcctcccggatggctccctcaaggtgggtcgtgtggcgcatttggtggtttcgttgggcataattctggccatcgacaaggccttgaaaaggctcttcacggctgccatgataaagttccctagcgcattgtttggaatgttctgcatattttccgtgctgtcaatcctcgacgtcatcagaccggcagcggcaacggctctactgaacttttttgagccggcaacaacgttcatacagagatggcttccgctcttctacgtgccgtcgctggtagtgttgcccttagctgtgaagggaatccctgcttcttccggaatgaagattggcgcaattcttgttggaggctgggcagcatcactctatgttgcagggtacacagccattgccgtaagaaagatggtcagaacaaaaatgatagcagcagagcccatgccaaagccttctccgttttcaacaatagagatttggttctggactattatcttgtcagtttcatttatattgactgttctttcacctcttgcatttggaacttatgctcgaacatgccttccgtgtcttttagcggcaactgcgttaggattcatccttggatctggatttccacctactgccaagaagctactacatccaattattacttgtgccctatctgcggatttggcagcctttgcttgtgggatctccacaggattgggctttgaatccacgctaggggcctatttaactaaaagtgcaaccaatcctggtgctggtgatatcttgatgggatttcttggatctgtgattctttcattcgctttctcaatgttccgtcagagaaagcttgtcaaacggcatgctgcggagatcttctcagctgtggtggtttcaacaattttttctctgtactcaacagcagctatgggccgaatgattggactggaaccaaccttaactgtttccattttgccaagatgtattactgttgcactagcattgagcattgtctcgctttttgatgggcaatataactctcgggcggattgtccttcagggtctagggatgaaagaaaatga

SEQ ID NO: 36 - DNA, 255 bp, synthetic construct
  source 1..255: mol_type = other DNA
  misc_feature 1..255: note = Plgg1 for RNAi
atggcttcatcccaccaattccaaacttcatggctgctcacacggagaccgttgggagacgcctctttgcgtttatatcaattaccccgcacgaccacaaaagagcagaggaaaaattatgacaattgcagctgttgggcgcagaggagaagagaggcagctgtcgctgcagcaatcagaattaatggtgagcgtaagttcatgttcacggggtttggcaggcaaaggccaaggcgaagtcatgaaataataatg

SEQ ID NO: 37 - DNA, 255 bp, synthetic construct
  source 1..255: mol_type = other DNA
  misc_feature 1..255: note = Plgg1 for RNAi
cattattatttcatgacttcgccttggcctttgcctgccaaaccccgtgaacatgaacttacgctcaccattaattctgattgctgcagcgacagctgcctctcttctcctctgcgcccaacagctgcaattgtcataatttttcctctgctcttttgtggtcgtgcggggtaattgatataaacgcaaagaggcgtctcccaacggtctccgtgtgagcagccatgaagtttggaattggtgggatgaagccat

SEQ ID NO: 38 - DNA, 1581 bp, Populus alba
  source 1..1581: mol_type = unassigned DNA
  misc_feature 1..1581: note = Plgg1
atggctactcctttagtcgctctttccgttcaactctgtcatcaccactcaaaacaatattctttcaaatcacaatcacaatcacgtgtcaatagggatttccgtacaaaaacactacttggtgtttacaatggattccaaaatgtatataaccaatcttattttcataagccttgggcacccattagagttcttgagcctaattcaaggttcttgcaaatgggtcctcaagaaacctgctctagtcgaggaatttccaagaaatctatgagctcagaaggcagtaccagtactagctcttcatctatttctcaacaggtgattgggatcttgcatttgcttgtttcacttgggattatccttgcaatggataagttgttgaaaaaggcatttgtggctgctgctatcaagtttccaagtgctctgtttggcatgttctgcatattctcagttttagtgattcttgatataactattccggctgctgcaacaagcttaatgaacttctttgagccagcactgttattcattcagagatggcttccgttgttctacgttccatcattggttgttttgcctctctctgttaaagatatccctgctgcatcaggtgtcaagatttgcttcatcacagctggagggtggctggcgtcactttgtgtggcgggtttcacagctattgctgtgagaaaaatggtgaagacagaaatgactgacgctgagcctatggcgaaaccctctcctttttctccattggaaatgtgggcttggagtggggtcttccttgtatcatttgttgtcgcattattatatagaacggcattggggactgcagccagaacatgccttcctttcctactagcgtccaccgtgctaggctatatggttggttctgggttaccatctggtgttaagaaggttttccatcccattatttgttgtgcactatctgcagatttggcagcgttggcctttgggtacctttcccaatctggacttgatcccgttctaggatattaccttacaaaagtttcatctaatcctggagctggtgatgtgttaatgggattcttgggacctgtcattctttcttttgccttctcaatgttcaagcagcgaaagcttgttaagagacatgcagctgagattttcacgtcggtcattgtttcaacactattttcattgtattcaacggctctcgtgggacgtctagttgggttagaaccaacgttgactgtatccattattcccagatgtataaccgtggcattagccctcagcattgtgtcattctttgaaggtgccaattcatctctcacagctgctgtagttgttgtaactggtctcattggagcaaattttgtacaagcagtgcttgataaattaagttttcgtgatcccattgctcgaggaatagctactgcttccagtgctcatggactgggaactgcagcattgtcagccaaggaacctgaggcacttccattttgtgccattgcttatgctctcactggtatatttggttcattgttttgttcagttcctgcagttaggcaaagcttacttgcaataattggctga

SEQ ID NO: 39 - DNA, 1575 bp, Populus tremula
  source 1..1575: mol_type = unassigned DNA
  misc_feature 1..1575: note = Plgg1
atggctactcctttagtcgctctttccgttcaactctgtcatcaccactcaaaacaatattctttcaaatcacaatcacgtgtcaatagggatttccgtacgaaaacactacttggtgtttacaatggattccaaaatgtatataaccaatcttattttcataagccttgggcacccattagagttcttgagcctaattcaaggttcttgcaaatgggtcctcaagaaacctgctctagtcgaggaatttccaagaaatctatgagctcagaaggcagtaccagtactagctcttcatctatttctcaacaggtgattgggatcttgcatttgcttgtttcacttgggattatccttgcaatggataagttgttgaaaaaggcatttgtggctgctgctatcaagtttccaagtgctctgtttggcatgttctgcatattctcggttttagtgattcttgatataactattccggctgctgcaacaagcttaatgaacttctttgagccagcactgttattcattcagagatggcttccgttgttctacgttccatcattggttgttttgcctctctctgttaaagatatccctgctgcatcaggtgtcaagatctgcttcatcacagctggagggtggctggcgtcactttgcgtggcgggtttcacagctattgctgtgagaaaattggtgaagacagaaatgactgacgctgagcctatggcgaaaccctctcctttttctccattggaaatgtgggcttggagtggggtcttccttgtatcatttgttgtcgcattattatatagaacggcattggggactgcagccagaacatgccttcctttcctactagcgtccaccgtgctaggctatatggttggttctgggttaccatctggtgttaagaaggttttccatcccattatttgctgtgcactatctgcagatttggcagcgttggcctttgggtacctttcccaatctggacttgatcccgttctaggatattaccttacaaaagtttcatctaatcctggagctggtgatgtgttaatgggattcttgggacctgtcattctttcttttgccttctcaatgttcaagcagcgaaagcttgttaagagacatgcagctgagattttcacgtcggtcattgtttcaacactattttcattgtattcaacggctctcgtgggacgtctagttgggttagaaccaacgttgactgtatccattattcccagatgtataaccgtggcattagccctcagcattgtgtcattctttgaaggtgccaattcatctctcacagctgctgtagttgttgtaactggtctcattggagcaaattttgtacaagcagtgctcgataaattaaattttcgtgatcccattgctcgaggaatagcaactgcttccagtgctcatggactgggaactgcagcattgtcagccaaggaacctgaggcacttccattttgtgccattgcttatgctctcactggtatatttggttcattgttttgttcagttcctgcagttaggcaaagcttacttgcaataattggctga

SEQ ID NO: 40 - DNA, 1578 bp, Populus euphratica
  source 1..1578: mol_type = unassigned DNA
  misc_feature 1..1578: note = Plgg1
atggctactcctttagtcgctcattccgttctactctgtcatcaccactcaaaacaatatcctttcaaatcgcaatcacgtgtcgatagggatttccgtacgaaaacactacttggtgtttacaatggattccaaaatgtatataaccaatcttattttcataagccttgggcacccattagagttcttgagcctaatttaaggttcttgcaaatgggtcctcaagaaacctgctctagtcgacgaatttccaagaaatctatgagctcagaaggcagtaccagtagtactagctcttcatctatttctcaacaggtgattgggatcttgcatttgcttgtttcacttgggattatccttgcaatggataagttgttgaaaaaggcatttgtggctgctgctattaagtttccaagtgctctatttggcatgttctgcatattctcagttttagtcattcttgatataactattccggctgctgcaacaagcttaatgaacttctttgagccagcactgttattcattcagagatggcttccattgttctacgttccatcattggttgttttgcctctctctattaaagatatccctgctgcatcaggtgtcaagatttgcttcatcacagctggagggtggttggcgtcactttgtgtggcgggttttacagctattgctgtgagaaaaatggtgaagacagaaatgactgatgctgagcctatggcgaaaccctctcctttttctccattggaaatgtgggcttggagtggggtcttccttgtatcatttgttgtcgccttattatatagaacggcattggggactgcagccagaacatgccttcctttcctactagcatccaccgtgctaggctatatggttggttctgggttaccatctggtgttaagaaggttttccatcccattatttgttgtgcactatctgcagatttggcagcgttggcctttgggttcctttcccaatccggacttgatcccgttctaggatattaccttacaaaagtttcatccaatcctggagctggtgatgtgttaatgggattcttgggatctgtcattctttcttttgctttctcaatgttcaagcagagaaagcttgttaagagacatgcagctgagattttcacgtcagtcattgtttcaacactattttcattgtattcaacggctctcgtgggacgcctagttgagttagaaccaacattgactgtatccattattcccagatgtataaccgtggcattagccctcagcattgtgtcattctttgaaggtgccaattcatctctcacagctgctgtagttgttgtaactggtctcattggagcaaattttgtacaagcagtgctcgataaattaaattttcttgatcccattgctcgaggaatagcaactgcttccagtgctcatggactgggaactgcagcattgtcagccaaggaacctgaggcacttccattttgtgccattgcttatgctctcactggtatatttggttcattgttttgttcagttcctgcagttaggcaaagcttacttgcaataattggctga

SEQ ID NO: 41 - DNA, 885 bp, Picea abies
  source 1..885: mol_type = unassigned DNA
  misc_feature 1..885: note = Plgg1
atggccctggaagttaaacctgctaaaaatagtaaattgccaaaattgccagaaaactattttgagccagtcggaaaaggtgggtggccactgatatcctcttggatcaaacggcatgctgcagagatcttctcagctgtggtggcttcaacaattttttctctgtactcaacaacagttgtaggtcgaatgattggaatggaaccaaccttaactgtttgcattttgccaagatgtattactgttgcattagcactgagcattgtctcactttttgatgctgcaaatccttcccttacagtagctgtcatggttttaacgggattggttggggaaaattttgcacaaatatttctcgacaagcttggtttggatgattctattgcacgaggaatggctatagccttcagtgctcatggattgggaacgacagcattgtcagcaaaagaaccagaagcattgcctttctgtgcaatttcctatgcattaactgcggaggccatggctggagatggtggatcggagcgccttgacgaagcgggagtcgctgggttaggcgagcaacaagatctgcaagaattgtacatatttccacatcgattacctattgctgctctgacggagcctctcgtcctactcaacaggacctacagcaacagggaggttctcgggatcatgacggtgctaaccattgttatcgtgtttctcacgagcattgggtcgttactcatctcggtgcttatggtcggattgggtatcgtttgtgtccatggtgcatttagggtttccgaagatctgtttttggacgagcaggagcaggcggtcagattcttgtccttcctcggtggcggtggatcccatccccctgttgctatccatgtttga

SEQ ID NO: 42 - DNA, 1903 bp, Populus trichocarpa
  source 1..1903: mol_type = unassigned DNA
  misc_feature 1..1903: note = malate synthase
gaaacactctcatacacgttaaaaaccatacaacaaagaagagtggcattgcttaaaaaacagtcagatcaataatgggacctgcaacttatgagaatcactatccaccgcccaatatgaagaagtttggctatgatgctcctgaaggagttgaaattagaggcagatatgacggagagtttgcagtgatccttaccaaggatgctttgcaatttgttgctgatttgcagagagagtttaggaaccgcatcaagtatgcaatggagtgtcgcaaggaagccaaaaggaggtacagtgagggggctttaccagggtttgatccagctacaaggtacataagggagggggagtggacatgtgcacctgtccctccagctgttgctgataggaaggtggagattacaggtccagtggagaggaaaatgatcatcaatgcactcaattctggagctaaagttttcatggctgactttgaagatgcactgtcaccaagttgggagaaccttatgagaggacaagttaacctgaaggacgctgtcgctgggactattacctttcacgacaaggtcaggaacagggtttataagctgaataaccagacagccaagttgtttgtccgcccacgaggttggcatctacctgaggcgcacattctcattgatggagaaccagcaactggttgccttgtggactttggcctttacttttaccacaactatgcagcattccgtcggattcagggtgcagggttcgggcctttcttctatctccctaagatggagaattcaagggaggctaagatatggaactgtgtgttcgagaaagcagagaagatggcaggaattgaaaggggaagcatcagggccactgtcctaatcgaaacacttccagctgttttccaaatgaatgaaatcctctacgaactaagggatcactctgttggcttgaactgcggaagatgggattacatcttcagctatgtcaagacattccaggctcacccagatcgcctgctaccagacagggttcaagttggcatgactcagcacttcatgaagagttactctgatctcctcatctggacatgccataggcgtggtgtccatgcgatgggcggcatggcagctcagattccaatcagagacgatccagaggcgaataatgaagcactagaactcgtgcgaaaggacaagctaagagaggttcgagcaggacatgatggaacatgggcagcccaccctggacttatcccagcttgcatggaagtcttctcaaacaacatgggaaacacgccaaaccaaatccagtccatgaagcgcgaagatgcatcaaccattacagaggaagacctcttacagaggccaagagggtcccgttcgttggaaggtctccgcctaaacacccgtgtaggaatccagtacttagcagcatggctaactggaactggctcagtccctctttacaacctcatggaagacgctgccactgcagaaatcagcagagttcagaactggcaatggcttaaatatggagtggaacttgacggagatggccttggagtgaaagttaacaatgatctgttcgggaaagtggtggaagaggaaatggctaggattgaaagggaagttgggaaagagaaattcaagaggggaatgtacaaggaggcctgcaagattttcgcaaggcaatgcacagcacctacactggatgattttcttaccctgaatgcttatgataatatcgtcatccatcaccctatgggatcatcatcccgcctctaagtcatgagtttcatcgcttgtttttaatttcctgggattgatgtgtgccctcgtgttctactctactactacaacttgaataaaatcctttctatctgtttgaacttaagttccacccatta

SEQ ID NO: 43 - DNA, 2038 bp, Populus tremula
  source 1..2038: mol_type = unassigned DNA
  misc_feature 1..2038: note = malate synthase
gtttaatcaaatcttacgcacataaagccataacgataatgctccttgtacctatccacttgcctccactctccctttatatatttctactcttgagcttacccttgaaacactctcatacacgttaaaaaccatacagtaaagaagaatcgcgttgctaaaaaaacagtgagatcaataatgagacctgcaactaatgggaatccggatgcaccgcccgatatgaagaagtttggctatgattctcctgaaggagttgaagttagaggcagatatgacggcgagtttgcagtgatccttaccaaggatgctttgcaatttgttgctgatttgcagagagagtttaggaaccgcatcaagtatgcaatggagtgtcgcaaggaagccaaaaggaggtacagtgagggggctctaccagggtttgatccagctacaaggtgcataagggaggaggagtggacatgtgcacctgcccctccagctgttgatgataggaaggtggagattacaggtccagtggagaggaaaatgatcatcaatgccctcaattctggagcgaaagttttcatggctgactttgaagatgcgctctcaccaagctgggagaaccttatgagaggacaagttaacctgaaggatgctgttgatgggactattacctttcacgacaaggtcaggaacagggtttataagctgaataatcagacagccaagttgtttgtccgcccacgaggttggcatctacctgaggcgcacattctcattgatggagaacctgcaactggttgccttgtggactttggcctttacttttaccacaaccatgcagcattccgtcggaatcagggtgcagggttcgggcctttcttctatctccctaagatggagaattcaagggaggctaagatatggaactgtgtgtttgagaaagcagagaaggtggcaggaattgaaaggggaagcatcagggccactgtccttatcgaaacacttccagctgtcttccaaatgaatgaaatcctccacgaactaagggatcactctgttgggttgaactgcggaagatgggattacatcttcagctatgtcaagacattccaggctcacccagatcgcctgctaccagacagagttcaagttggcatgactcagcacttcacgaagagctactccgatctcctcatctggacatgccataggcgtggtgtgcacgcgatgggcggcatggcagctcagattcctatcagagacgatccagaggcgaataaggaagcactggagctcgtgcgaaaggacaagctaagagaggttcgagcaggacatgacggaacatgggcagcccaccctggacttatcccagcttgcatggaagtcttctcagacaacatgggaaacacgccaaaccagatccaaaccatgaagcgcgaagatgcatcaaccataacagaggaagacctcttacagagaccaagagggtcccgttcattggaaggtctccgcctaaacacccgtgtgggaatccagtacctagcagcatggctaactggaactggatcggtccctctttacaacctcatggaagatgccgccaccgcagaaatcagcagagttcagaactggcaatggcttaaatatggagtggaacttgacggagatggccttggagtgagagttaacaatgatctgttcgggaaagtggtggaggaggagatggctaggattgaaagggaggtcgggaaagagaagttcaagaggggaaggtacaaggaggcctgcaagattttcgcaaggcaatgcacagcacctacactagatgattttcttacactgaatgcttatgaaaatatcgtcagccatcaccctatgggatcatcaccccgcctctaagtcacgagtttcatcgcttgtttttaatttccagggattgatgtgtgccctcgtgttccactctactactacaagttatctacaacttgaataaaatcttttctacctgtttgaacttaagttccaccctttacttgcacttattttattt

SEQ ID NO: 44 - DNA, 1713 bp, Eucalyptus grandis
  source 1..1713: mol_type = unassigned DNA
  misc_feature 1..1713: note = Malate Synthase
atgatggatctcgggtcgttcacgtaccaagcaccagcaaccaacaaggccaaccccgcgtccggctacgacgtccccgagggagtggacatcagagggcgttacgatgcccagttcgccaggatcctcnaccgggacgccttgcagttcgtcgccgatctgcagagggagttcaggggccacatcaagtacgccatggagtgtcgccgcaacgccaagcagcgctacaacgccggcgcgctgcctgggttcgaccccgctacgaggtacgtgagggaaggcgagtggacgtgctcgctggtcccaccagccgtggccgaccggagggtggagatcactggccctgtcgagcgcaagatgatcatcaacgcgctcaactctggcgccaaagttttcatggcagatttcgaagatgcgctatccccgagctgggagaacctgatgagagggcaagtgaacctgaaggatgcggtggatgggaccataaccttccacgacaaggcccgaaacagggtctacaagctcaatgaccagattgccaagctctttgtccggccccgaggttggcacttgcctgagtctcacatcctcattgacggtgagcctgcaaccggttgcctcttcgactttggcctctacttcttccacaactacgcttcattcaggaaaatgcagggtgaggggttcgggcccttcttctatctccctaagatggagcattccagggaagcgaagatatggaacagcgtctttgagagggcagagaaaatggcgggcatcgagagagggagcataagggcaacggtattgatagagacactacctgcagtgttccagatggatgagatactgtacgagctgagggatcactcggtcggattgaactgcggaagatgggactacatcttcagctatgtcaagaccttccaggctcatcccgatcgcctactgcctgacaggtttcaggtcgggatgacccagcacttcatgaggtcttactctgatctcctcatccggacttgccatcggcgcggtgttcatgccatgggaggcatggcggctcaaatcccgatcagagatgatccggtggccaatgaggcggcgctggaactagtaaggaaagacaagctcagagaagtcagggccgggcatgacgggacatgggctgctcacccggggttaatcccggcttgtatggaagtcttcacgaatcacatgggcaatgcgccacaccagatccaatcagtcaagagagaggacgcagccaacataaccgaggaagacctcctccagaggccgagaggggcccgaactttggaaggcctccggctcaacactcgggttgggatccaatacctggcagcctggctcacagggacagggtcggtcccactctacaatctcatggaggatgcggcaactgccgagatcagccgagtccagaactggcagtggctgaagtatggtgtggagctggacggtgatggggttggagtgagagtgagcttggatctattcgcgagggtggtggaggaagaggtggccaggattgagnnagaggttgggaaggacaagttcaagaaggggatgtacaaggaggctggcaagatgttcacgagacaatgcacggcgccagtgttggacgatttcctgacgctcgatgcctacaatcacatcgtcgtgcatcatcctagggggtcttccaagctctga

SEQ ID NO: 45 - protein, 568 aa, Eucalyptus grandis
  REGION 1..568: note = Malate Synthase
MMDLGSFTYQAPATNKANPASGYDVPEGVDIRGRYDAQFARILRDALQFVADLQREFRGHIKYAMECRRNAKQRYNAGALPGFDPATRYVREGEWTCSLVPPAVADRRVEITGPVERKMIINALNSGAKVFMADFEDALSPSWENLMRGQVNLKDAVDGTITFHDKARNRVYKLNDQIAKLFVRPRGWHLPESHILIDGEPATGCLFDFGLYFFHNYASFRKMQGEGFGPFFYLPKMEHSREAKIWNSVFERAEKMAGIERGSIRATVLIETLPAVFQMDEILYELRDHSVGLNCGRWDYIFSYVKTFQAHPDRLLPDRFQVGMTQHFMRSYSDLLIRTCHRRGVHAMGGMAAQIPIRDDPVANEAALELVRKDKLREVRAGHDGTWAAHPGLIPACMEVFTNHMGNAPHQIQSVKREDAANITEEDLLQRPRGARTLEGLRLNTRVGIQYLAAWLTGTGSVPLYNLMEDAATAEISRVQNWQWLKYGVELDGDGVGVRVSLDLFARVVEEEVARIEEVGKDKFKKGMYKEAGKMFTRQCTAPVLDDFLTLDAYNHIVVHHPRGSSKL

SEQ ID NO: 46 - DNA, 3288 bp, Chlamydomonas reinhardtii
  source 1..3288: mol_type = unassigned DNA
  misc_feature 1..3288: note = Glycolate Dehydrogenase
atgccacgcggccagggcaagcgcctggctcagctccttggagctcagctgaagcagtacgcagcggaggtgcgtggcatcagcacagctggtggcgcttctcgcggtggagctcgaggacctgcatcccctagctcgctagagcagcagacgcgccaggtcgctcaggttgctgttcagcagtcgactcagcaggcagtgaaggtcgttgtgccggccatcaaagtagacctggttggtgcggtcagctcggtgtctgagagcgacaaggtggagccgggtgtgttcaagaacgtggatggccaccgcttcgaggacggtcgctatgccgcttttgttgaggagattacaaagtttatccccaaggagcgccagtactcggaccccgtgcgcacattcgcgtatggcacggatgcctccttctaccggcttaacccgaagctggtagtgaaggtgcacaacgaggacgaggtccgccgcatcatgcccatcgcggagcggctgcaggtccctatcaccttccgcgcggccggcacgtcgctgtctgggcaggcaattaccgactcggtgctcattaagctgagccacacgggcaagaacttccgcaactttaccgtgcacggcgacggtagcgtgatcacggtggagccgggcctcattggcggcgaggtgaaccgcatcctggcggcacaccagaagaagaacaagctgcccatccagtacaagatcggacccgacccctcctccatcgacagctgcatgatcggcggcatcgtgtccaacaacagcagcggcatgtgttgcggcgtgagccagaacacctaccacacgctgaaggacatgcgggtggtgttcgtagacggaacggtgctggacacggccgaccccaactcgtgcaccgccttcatgaagagccaccgctcgctggtggatggcgtcgtgagcctggcgcgccgcgtgcaggccgacaaggagctgacggcgctcatccgccgcaagttcgccatcaagtgcaccaccggctactccctgaacgcgctggtggacttcccggtggacaaccccattgagatcatcaagcacctcatcatcggcagcgagggcacgctgggcttcgtcagccgcgccacctacaacaccgtgcccgagtggcccaacaaggcctcggccttcatcgtgttcccggacgtgcgcgccgcctgcaccggcgcctcggtgctgcgcaacgagacgtccgtggacgcggtggagctgtttgaccgcgccagcctgcgcgagtgcgagaacaacgaggacatgatgcgcctggtgcccgacatcaagggctgcgaccccatggcggcagcgctgctgatcgagtgccgcggccaggacgaggccgcactgcagagccgcattgaggaggtggtgcgcgtgctgacggcggcgggcctgcccttcggcgccaaggccgcgcagcccatggccatcgacgcctaccccttccaccacgaccagaagaacgccaaggtcttctgggacgtgcgcaggggcctgatccccattgtgggcgcggcgcgcgagcccggcacatccatgctgatcgaggacgtggcctgccccgtggacaagctggccgacatgatgatcgacctgatcgacatgttccagcgccacggctaccacgacgcctcctgcatcggccacgcgctcgagggcaaccttcacttggtgttctcgcagggcttccgcaacaaggaggaggtgcagcgcttcagcgacatgatggaggagatgtgccacctggtggccaccaagcactcgggcagcctcaagggcgagcacggcacgggccgcaacgtggcgccgttcgtggagatggagtggggcaacaaggcgtacgagctgatgtgggagctcaaggcgctgttcgaccccagccacaccctcaacccgggcgtcatcctcaaccgcgatcaggacgcgcacatcaagttcctgaagccctcgcccgcggcctcgcccatcgtcaaccgctgcatcgagtgcggcttttgcgagtccaactgcccctcgcgcgacatcacgctcacgccgcgccagcgcatctccgtgtaccgcgagatgtaccgcctcaagcagctgggcccgggcgccagcgaggaggagaagaagcagctggcggccatgagcagctcgtacgcctacgacggcgagcagacgtgcgcggcggacggcatgtgccaggagaagtgccccgtcaagatcaacacgggagacctgatcaagtcgatgcgtgccgagcacatgaaggaggagaagaccgccagcggcatggcagactggctggccgccaacttcggcgtcatcaactccaacgtgccgcgcttcctcaacatcgtcaacgccatgtacagcgtggtgggctcggcgcctctgtccgccatcagccgcgcgctcaacgccgccaccaaccacttcgtgccggtgtggaacccctacatgcccaagggcgcggcgccgctcaaggtgcccgccccgccggcgccggcagctgctgaggcctcgggcatcccgcgcaaggtggtgtacatgtccagctgcgtgacgcgcatgatgggccccgccgcctccgacaccgagacggcggcggtgcacgagaaggtgatgagcctgttcggcaaggccggctacgaggtgatcatccccgagggcgtggccagccagtgctgcggcatgatgttcaacagccgcggcttcaaggacgccgccgccagcaagggcgcggagctggaggcggcgctgctcaaggcctcggacaatggcaagatccccatcgtcatcgacacctcgccctgcctggcgcaggtgaagagccagatcagcgagccgtcgctgcgcttcgcgctgtacgagccggttgagttcatccggcacttcctggtggacaagctggagtggaagaaggtgcgcgaccaggtggccatccacgtgccctgctcctccaagaagatgggcatcgaggagtccttcgcgaagctggcgggcctgtgcgccaacgaggtggtgccctcgggcattccttgctgcggcatggcgggcgaccgcggcatgcgcttccccgagctgaccggcgcctcgctgcagcacctcaacctgcccaagacctgcaaggacggctactccaccagccgcacctgcgagatgtcgctcagcaaccacgccggcatcaacttcaggggcctggtgtacctggtggatgaggccacggcgcctaagaagcaggccgccgctgccaagaccgcgtaa

SEQ ID NO: 47 - protein, 1095 aa, Chlamydomonas reinhardtii
  REGION 1..1095: note = Glycolate Dehydrogenase
MPRGQGKRLAQLLGAQLKQYAAEVRGISTAGGASRGGARGPASPSSLEQQTRQVAQVAVQQSTQQAVKVVVPAIKVDLVGAVSSVSESDKVEPGVFKNVDGHRFEDGRYAAFVEEITKFIPKERQYSDPVRTFAYGTDASFYRLNPKLVVKVHNEDEVRRIMPIAERLQVPITFRAAGTSLSGQAITDSVLIKLSHTGKNFRNFTVHGDGSVITVEPGLIGGEVNRILAAHQKKNKLPIQYKIGPDPSSIDSCMIGGIVSNNSSGMCCGVSQNTYHTLKDMRVVFVDGTVLDTADPNSCTAFMKSHRSLVDGVVSLARRVQADKELTALIRRKFAIKCTTGYSLNALVDFPVDNPIEIIKHLIIGSEGTLGFVSRATYNTVPEWPNKASAFIVFPDVRAACTGASVLRNETSVDAVELFDRASLRECENNEDMMRLVPDIKGCDPMAAALLIECRGQDEAALQSRIEEVVRVLTAAGLPFGAKAAQPMAIDAYPFHHDQKNAKVFWDVRRGLIPIVGAAREPGTSMLIEDVACPVDKLADMMIDLIDMFQRHGYHDASCIGHALEGNLHLVFSQGFRNKEEVQRFSDMMEEMCHLVATKHSGSLKGEHGTGRNVAPFVEMEWGNKAYELMWELKALFDPSHTLNPGVILNRDQDAHIKFLKPSPAASPIVNRCIECGFCESNCPSRDITLTPRQRISVYREMYRLKQLGPGASEEEKKQLAAMSSSYAYDGEQTCAADGMCQEKCPVKINTGDLIKSMRAEHMKEEKTASGMADWLAANFGVINSNVPRFLNIVNAMYSVVGSAPLSAISRALNAATNHFVPVWNPYMPKGAAPLKVPAPPAPAAAEASGIPRKVVYMSSCVTRMMGPAASDTETAAVHEKVMSLFGKAGYEVIIPEGVASQCCGMMFNSRGFKDAAASKGAELEAALLKASDNGKIPIVIDTSPCLAQVKSQISEPSLRFALYEPVEFIRHFLVDKLEWKKVRDQVAIHVPCSSKKMGIEESFAKLAGLCANEVVPSGIPCCGMAGDRGMRFPELTGASLQHLNLPKTCKDGYSTSRTCEMSLSNHAGINFRGLVYLVDEATAPKKQAAAAKTA

SEQ ID NO: 48 - DNA, 4680 bp, Volvox carteri
  source 1..4680: mol_type = unassigned DNA
  misc_feature 1..4680: note = Glycolate Dehydrogenase
atgacagctatgcaggccaacggcggcagcggcattagcgatgttactgccaccgccgcgaccacgggggagacctcaggtggggaagcggggccagtgggacggctggaggcggccacggcgccaacggacgcggcggcgctggcctactcagatgcgggtgccaccggcgtagacggcctagatgagacgcgcaagtttgtgttcgcggaggaatggggcttttcgcgcgtgggaggtgacctgccggatggcaccacgcccgccacgtttgccgacatgtttcccgagcgtttcttcgccttcaaccccctgcgcgcggtaacggcggtggcgctaccactggtcgctatggccgttggctacggctggctgtggtacatgcactcaatctgccccgcctggcagcagctcgcgtgcgcagcccttatcggcaccgcttacacgggcctctttaaagtcgcccacgagtgtgcgcgcttcagctttctgcctgaagctcctcgcctccaagatgcgctagggcttgtcctcatgctgccgtcgttgtaccccttcacctcctggcgcctccattacatccaccatatggcgcacctcaacatgctgtgggaggatacctttggctggcacccgtacacgaagctgcagctggccacggagctcctctgtgggcagcactggctcctggcactgcggcgcctggcgctgaccacaccgctaaagctgtttgcgtccgtgggccactggcttcgctcctttgacgggctggacttgaagcgcttccacaaagacacctactgggctgtgctggcgggctggtctgggcccatcctgtttttgggtttcggcatcccggctatcattaagatgtttggcttgtcagtccgtctagccgaccgggcttgggggggcttagtaagcggctacctgcttccctggctggtcttccacttctggctgtccactctatcgctcctgcagcacaccgcaccgcatattccattccgcgccgaggacgcgggctacgactccggccgcgcggccatctgcggcaccgtgacggtgcggctgccacggcctctggagctgctgcttaacgacgccaactactccctaccgcagctggtggcccctgggatgccagtgtttcacgcgcgggaggcgtatgagtacatgcgggagcggttgctgccgtacctgacggaggcgacgcttagcgtaaaactactgaccaaccatattaccaagtggcaggtttatgatgcggaaaaggagacgtaccttcctctggatgaggtcatgcaggagctcgaacgcgacattgccactcttatggaggaggctgagcaggcggaggaggaggagaaagtttcaggggggccaagtggcggagcggtagaggggcagggcaagcgcctggcgcagctgttgggtgcgcagcttaagcaatacatttcggaggttcgcagcatcagcagctcaaatgctgctggtcggggagcgttgggcagcggccacacgcctacgacgcaggtcgtcactggccagcaaacacgctctcatgcccaggtcgcagtccagcaggctacacagcagcacttgaaagttgtggtgccggccatcaggaccgatctggtcggagcggtgtctatggtgtctgatggcgagaaagtggagccaggcgtcttcaagaacattgacggacaccgcttcgacgacggtcgctaccaggcttttattgaggaaatcacaaaatttatccccaaggagcggcagtacaccgaccctgtccgcacgttcgcgtatggcatcgacgcgagtttctaccgccttaacccaaagctggtcgttaaggtccacaatgaagaagaagttcggaagattttgcccatcgcggagcggctcaaggtgccggtcaccttccgtgctgcgggtacatccctctcgggccaggctatcaccgactcggtgctgattaagctcagccacaccggcaagaatttccggaactacaccgtgcacggcgacggcagcagcatcacagtggagccgggcctcatcggtggcgaggttaaccgcatcctggcggcgtatcagaagaagcacaagctgcccattcagtacaagataggcccagacccgtcttccatcgactcttgcatgatcggcggcatcgtggccaacaacagcagcggcatgtgctgcggtgtcagccaaaacacatatcacacgctcaaggatatgcgggtgttgtttgtggacggcacggtactggatacggcagatccggacagctgcgcggccttcctcaagagccataaggccctggtggacggcgtcgtggacctggcgcggcgtgtgcaggctgacagggagctgacggctcttatccgtcgcaagtttgccatcaaatgcaccaccggctattcgctgaatgcgctggtggatttcccgctggacaaccccattgagattatcaagcacgtcatcattggcagcgagggcactctgggcttcgtgtcgcgggccacctacaacactgtgcccgagtggcccaacaaggcctcggccttcatcgtcttcccggacgtgcgggcggcatgcatgggtgcctccgtgctccgaaatgagacctctgtggacgcagtagagctgtttgacagggcttccctacgcgagtgcgagaacaatgaggacatgatgcggctggtgccagacatcaagggctgcgaccccatggctgccgcgctgctgattgagtgccgtggccaggacgaggcggcgctgcagggtcgtattgaggaggtggtccgtgctcttacctcggcgggcctgccggtgggagcgaaggcggctgaaccccgccccatcaccgcctatcccttccaccacgatgccaagaactcgaaagtcttctgggacgtgcgcaggggtctgattccgatcgtgggtgccgcccgtgagccgggtacctccatgcttattgaggacgtcgcttgccccgtggacaagctggcggacatgatgattgacctgattgacatgttccagcgctatggctacaacgacgcttcgtgcttcggccacgcgctggagggcaacctgcacctggtcttctctcagggcttccgtaccaaggaggaggtacagcgatttgcggacatgatggaggagatgtgctacatcgtggccactaaacacagcggcagcctgaagggcgagcatggcacaggccgcaacgtggctcccttcgttgagatggaatggggcagcaaggcgtatgagctgatgtgggagctcaaggagctgtttgaccccaagtacacgctcaaccccggcgtcattctcaaccgggaccccgatgcgcacatcaaattcctcaagccttcgcctgctgcttcgccaatcgttaatcgctgcatcgagtgcggcttctgcgagtccaactgcccctcccgggacatcacgctcacgccccgccagcgcatcgccgtgtaccgtgagatgtaccgcctgaagcagctgggcccggccgccagcgaggaagagaagcagcagttggcagccattacaagctcctatgcttacgatggcgagcagacgtgcgccgccgacggcatgtgccaggagaagtgccccgtcaagataaacacaggcgacatgatcaaggctctccgcgccgagcacatgaaggaggccaagtccgccagtggtgctgccatgtggatggctaacaacttcggagtcctcaacgccacagtgccgcgcttcctgaacctcgttaatttggcgcacagtatcctgggccccaagccgcttgaggccatctcccgggcgctgaacgctgccaccaatcacctggtgcccgtctggaacccctacatgcccaagggcgccgcgccgctgaaggtgcccgctccgttccctgcacccgcggcgtcctctggcggcatcccccgccgagtggtgtacatgccctcttgcgtgacccgcatgatgggccccgcggcgagcgacaaggagacggcctctgtgcatgagaagattttgagcctcttcaacaaggccggctacgaggtcattgttccagagggtgtcagcagccaatgttgcggcatgatgttcaacagccgtggcttcaaggacgccgctgccgccaagggtgccgatctggaagcggcgctgctcaaggcatccgataacggcaagatccccatcgttatggacacttcgccatgcctggcgcaggttaagagccaaatctcggagccagcgctacgcttcgcgctgtacgaaccggttgagttcatcaggcacttcctggttgacaaacttgagtggcgcaaggtgcgggagcaggtggccatccatgtgccctgctcatccaagaagatgggcatcgaggagtccttcgccaagctggcaggattgtgtgcgcacgaggttgtcccgtcgggcattccctgctgcggtatggctggcgaccgtggaatgcgctacccggagctgactggctcctcactgcagcacctaaacctgcccaagagctgcagcgatggctactccaccagtcgcacctgcgagatgtcgttgtccaaccactcgggcatcaacttcaggggcctggtctacttggtggacgaggcgactagccccaagaaggcggcaaccgcatga

SEQ ID NO: 49 - protein, 1559 aa, Volvox carteri
  REGION 1..1559: note = Glycolate Dehydrogenase
MTAMQANGGSGISDVTATAATTGETSGGEAGPVGRLEAATAPTDAAALAYSDAGATGVDGLDETRKFVFAEEWGFSRVGGDLPDGTTPATFADMFPERFFAFNPLRAVTAVALPLVAMAVGYGWLWYMHSICPAWQQLACAALIGTAYTGLFKVAHECARFSFLPEAPRLQDALGLVLMLPSLYPFTSWRLHYIHHMAHLNMLWEDTFGWHPYTKLQLATELLCGQHWLLALRRLALTTPLKLFASVGHWLRSFDGLDLKRFHKDTYWAVLAGWSGPILFLGFGIPAIIKMFGLSVRLADRAWGGLVSGYLLPWLVFHFWLSTLSLLQHTAPHIPFRAEDAGYDSGRAAICGTVTVRLPRPLELLLNDANYSLPQLVAPGMPVFHAREAYEYMRERLLPYLTEATLSVKLLTNHITKWQVYDAEKETYLPLDEVMQELERDIATLMEEAEQAEEEEKVSGGPSGGAVEGQGKRLAQLLGAQLKQYISEVRSISSSNAAGRGALGSGHTPTTQVVTGQQTRSHAQVAVQQATQQHLKVVVPAIRTDLVGAVSMVSDGEKVEPGVFKNIDGHRFDDGRYQAFIEEITKFIPKERQYTDPVRTFAYGIDASFYRLNPKLVVKVHNEEEVRKILPIAERLKVPVTFRAAGTSLSGQAITDSVLIKLSHTGKNFRNYTVHGDGSSITVEPGLIGGEVNRILAAYQKKHKLPIQYKIGPDPSSIDSCMIGGIVANNSSGMCCGVSQNTYHTLKDMRVLFVDGTVLDTADPDSCAAFLKSHKALVDGVVDLARRVQADRELTALIRRKFAIKCTTGYSLNALVDFPLDNPIEIIKHVIIGSEGTLGFVSRATYNTVPEWPNKASAFIVFPDVRAACMGASVLRNETSVDAVELFDRASLRECENNEDMMRLVPDIKGCDPMAAALLIECRGQDEAALQGRIEEVVRALTSAGLPVGAKAAEPRPITAYPFHHDAKNSKVFWDVRRGLIPIVGAAREPGTSMLIEDVACPVDKLADMMIDLIDMFQRYGYNDASCFGHALEGNLHLVFSQGFRTKEEVQRFADMMEEMCYIVATKHSGSLKGEHGTGRNVAPFVEMEWGSKAYELMWELKELFDPKYTLNPGVILNRDPDAHIKFLKPSPAASPIVNRCIECGFCESNCPSRDITLTPRQRIAVYREMYRLKQLGPAASEEEKQQLAAITSSYAYDGEQTCAADGMCQEKCPVKINTGDMIKALRAEHMKEAKSASGAAMWMANNFGVLNATVPRFLNLVNLAHSILGPKPLEAISRALNAATNHLVPVWNPYMPKGAAPLKVPAPFPAPAASSGGIPRRVVYMPSCVTRMMGPAASDKETASVHEKILSLFNKAGYEVIVPEGVSSQCCGMMFNSRGFKDAAAAKGADLEAALLKASDNGKIPIVMDTSPCLAQVKSQISEPALRFALYEPVEFIRHFLVDKLEWRKVREQVAIHVPCSSKKMGIEESFAKLAGLCAHEVVPSGIPCCGMAGDRGMRYPELTGSSLQHLNLPKSCSDGYSTSRTCEMSLSNHSGINFRGLVYLVDEATSPKKAATA